(19) United States
(12) Patent Application Publication
(10) Pub. No.: US 2026/0110025 A1
(43) Pub. Date: Apr. 23, 2026

(54) METHODS AND SYSTEMS FOR LONG-RANGE METHYLATION PROFILING

(71) Applicant: Moonwalk Biosciences, Inc., South San Francisco, CA (US)

(72) Inventors: Arash JAMSHIDI, South San Francisco, CA (US); Justin K. VALLEY, South San Francisco, CA (US)

(73) Assignee: Moonwalk Biosciences, Inc., South San Francisco, CA (US)

(21) Appl. No.: 19/144,874

(22) PCT Filed: Dec. 29, 2023
(86) PCT No.: PCT/US2023/086497

§ 371 Date: Jun. 30, 2025

Related U.S. Application Data
(60) Provisional application No. 63/436,295, filed on Dec. 30, 2022.

(51) Int. Cl. C12Q 1/6869; C12N 9/22; C12N 15/11; C12N 15/90

(52) U.S. Cl. CPC C12Q 1/6869; C12N 9/226; C12N 15/11; C12N 15/907; C12N 2310/20

(57) ABSTRACT

Methods for determining an epigenetic profile of a cell in a cell population are described herein. The method can include sequencing DNA molecules obtained from cells in a. cell population to provide a plurality of sequence reads comprising a methylation status for a plurality of bases in each sequence read; and assembling a plurality of contigs based on the plurality of sequence reads. Sequence reads having the same nucleobase sequence and methylation statuses within overlapping portions are joined together to form the same contig. Contigs having substantially the same nucleobase sequence and different methylation profiles are identified as being associated with different cells in the cell population.

### CROSS REFERENCE

This application claims the benefit of U.S. Provisional Application No. 63/436,295, filed Dec. 30, 2022, which is incorporated herein by reference in its entirety.

### SEQUENCE LISTING

The instant application contains a Sequence Listing which has been submitted electronically in XML format and is hereby incorporated by references in its entirety. Said XML copy, created on Dec. 26, 2023, is named 65120-708_601_SL.xml and is 31,075 bytes in size.

### TECHNICAL FIELD

Methods for long-range methylation sequencing and assembly are described herein, including methods of obtaining long-range methylation sequencing data indicative of single cells in a cell population.

### BACKGROUND

Certain epigenetic markers have been correlated to age or disease states in humans and other animals. Other epigenetic markers have been correlated to cellular identity. Since the discovery of Yamanaka factors (e.g., OCT4, SOX2, KIF4, and c-MYC), multiple studies have demonstrated the possibility to reverse aging and age-associated diseases through epigenetic reprogramming. Attempts to directly control cellular state through epigenomic editing is limited, for example by relying on few simultaneous targets (e.g., 1-2 CpG sites) and effector functions (methylation and/or demethylation). Further, techniques for modifying epigenetic markers to reverse causal biological aging markers can result in undesired loss of cellular identity.

Tracking epigenetic changes of a cell can aid cellular reprogramming protocols to ensure that the desired combination of epigenetic modifications is obtained. Recent advances in long-read DNA sequencing technology have made it possible to directly read out methylation over longer (thousands or millions of bases) DNA fragments. This sequencing data, however, is typically considered as individual sequence reads.

There remains a need to understand the complete epigenetic profile of a cell in a population of cells undergoing cellular reprogramming.

### SUMMARY OF THE DISCLOSURE

Described herein is an epigenetic profiling method for determining a methylation profile indicative of single cells in a cell population. The sequencing method can include obtaining DNA molecules from a cell population; sequencing the DNA molecules to provide a plurality of sequence reads comprising a methylation status for a plurality of bases in each sequence read; and assembling a plurality of contigs based on the plurality of sequence reads using sequence information (i.e., nucleobase sequence) and methylation status for the sequence reads. Sequence reads having the same nucleobase sequence and methylation statuses within overlapping portions can be joined together to form the same contig. Contigs having substantially the same nucleobase sequence and different methylation profiles are identified as being associated with different cells in the cell population. In some implementations, contig assembly may include matching a nucleobase sequence and methylation statuses in overlapping portions of at least two (e.g., at least three, at least four, or at least five) sequence reads. In some implementations, the sequence reads in the plurality of sequence reads are about 1000 to about 100,000 bases in length.

The sequencing may comprise a direct determination of methylation status for bases in the DNA molecules. For example, the sequencing may include a nanopore sequencing method. In another example, the sequencing comprises a determination of methylation status based on polymerase kinetics. The sequencing method need not include bisulfite treatment of the DNA molecules. The sequencing may include directly determining a 5mC status, a 5hmC statis, or a 6 mA status of one or more bases in the DNA molecules.

The assembled contig may include a substantially complete (e.g., 95% or more) chromosome.

Assembling the plurality of contigs may include using the methylation status for a plurality of CpG sites in the sequence reads.

In some implementations, assembling a contig in the plurality of contigs comprises matching a nucleobase sequence and methylation statuses in overlapping portions of at least two sequence reads.

In some implementations, assembling a contig in the plurality of contigs can include matching a sequence and methylation statuses in in overlapping portions of at least three sequence reads.

In some implementations, cells in the cell population are undergoing cellular reprogramming or have been subject to cellular reprogramming.

The cellular reprogramming may include, for example, contacting the cells with one or more cellular reprogramming factors that modify one or more epigenetic markers. The one or more cellular reprogramming factors may target one or more epigenetic markers. For example, in some implementations, the one or more cellular reprogramming factors that target the one or more epigenetic markers are targeted using a nuclease-deficient targeted DNA binding protein. The one or more cellular reprogramming factors that target the one or more target epigenetic markers may be targeted using a CRISPR-based editing platform. For example, the CRISPR-based editing platform of the one or more cellular reprogramming factors may include one or more single guide RNA (sgRNA) molecules that targets one or more epigenetic markers. In some implementations, the CRISPR-based editing platform of the one or more cellular reprogramming factors comprises a dead Cas9 endonuclease. In some embodiments, the nuclease-deficient targeted DNA binding protein of the one or more cellular reprogramming factors comprises a transcription activator-like (TAL) effector DNA-binding domain or a zinc finger DNA binding domain.

In some implementations, the one or more cellular reprogramming factors comprises one or more non-targeted cellular reprogramming factors.

For example, the one or more non-targeted cellular reprogramming factors may include one or more transcription factors. In some implementations, the one or more transcription factors comprises one or more Yamanaka factors. In some implementations, the one or more non-targeted cellular reprogramming factors comprises a high potassium medium.

In some implementations, the one or more cellular reprogramming factors comprises an epigenetic modification enzyme or an effector that recruits an epigenetic modification enzyme. Exemplary cellular reprogramming factors can include KRAB, VPR, p65 VP64, HSF1, p300, DNMT3A, TET1, EZH2, G9a SUV39H1, HDAC3, LSD1, PRDM9, DOT1L, FOG1, BAF, PYL1, ABI1, CIBN, ADAR2, METTL3, METTL14, ALKBH5, or FTO, or an active fragment thereof.

In some implementations, the cellular reprogramming comprises contacting the cells with a blocking reagent that specifically binds to one or more epigenetic markers. The blocking reagent may comprise, for example, a nuclease-deficient targeted DNA binding protein. In some implementations, the blocking reagent comprises nuclease-deficient targeted DNA binding protein that does not comprise a cellular reprogramming factor. For example, the blocking reagent may comprise a nuclease-deficient CRISPR-based editing platform. In some embodiments, the CRISPR-based editing platform of the blocking reagent comprises one or more single guide RNA (sgRNA) molecules that targets one or more epigenetic markers. In some embodiments, the CRISPR-based editing platform of the blocking reagent comprises a dead Cas9 endonuclease. In some embodiments, the nuclease-deficient targeted DNA binding protein of the blocking reagent comprises a transcription activator-like (TAL) effector DNA-binding domain or a zinc finger DNA binding domain.

In some implementations, cells in the population of cells are obtained from a cell line. In some implementations, cells in the population of cells are obtained from a tissue sample from an individual.

In some implementations, the cells in the population of cells comprise fibroblasts, keratnocytes, peripheral mononuclear blood cells, hepatocytes, neural cells, blood cells, immune cells, lung cells, pancreatic beta cells, cardiomyocytes, oligodendrocytes, or epithelial cells. For example, in some implementations, the cells in the population of cells comprise pancreatic beta cells or pancreatic alpha cells.

Also described herein is a method of evaluating a cell, comprising obtaining an epigenetic profile for the cells in the cell population according to the above method; and determining a differential between the obtained epigenetic profile and a target epigenetic profile. The target epigenetic profile may comprise one or more target epigenetic markers, and the one or more cellular reprogramming factors may target the one or more target epigenetic markers. The one or more target epigenetic markers may comprise an epigenetic marker associated with a biological age or a disease state.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings.

FIG. 1 shows an exemplary method for assembling sequence reads into a contig based on sequence information (i.e., nucleobase sequence) and methylation status for the sequence reads, according to some embodiments.

FIG. 2 shows the assembly of different contigs each indicative of different single cells in a cell population.

FIG. 3A shows an exemplary method for partially reprogramming a cell, according to some embodiments.

FIG. 3B shows an exemplary method for partially reprogramming a cell, which includes at least partially rejuvenating the cell, according to some embodiments.

FIG. 4 depicts an exemplary device, in accordance with some embodiments.

FIG. 5 depicts an exemplary system, in accordance with some embodiments.

FIG. 6 shows comparison of actual and reference null data sets for TCF7. Columns are CpGs in TCF7, rows are individual fragments spanning TCF7. Dark gray indicates methylated state. Light gray indicates unmethylated state.

FIG. 7 shows a plot of the Gap Statistic versus cluster number for TCF7. Dotted line indicates optimal number of clusters as given by: min(k) s.t. Gap(k)>=Gap(k+1)−3*SE(k+1), wherein min(k): minimum cluster number, k; Gap(k): Gap statistic at cluster number, k; Gap(k+1): Gap statistic at cluster number, k+1; SE(k+1): Standard error of the null distribution at cluster number, k+1.

FIG. 8 shows a heatmap of TCF7 showing optimal number of clusters based on the Gap Statistic. Row annotation (gray) are CpG annotations showing various transcripts from the UCSC database (increasing gray bar height corresponds to introns, promoters, and exons, respectively). Dark gray indicates methylated state. Light gray indicates unmethylated state,

FIGS. 9A-9Z and FIGS. 9AA-9HH illustrate heatmaps of various T cell related genes showing optimal number of clusters based on the Gap Statistic. Row annotation (gray) are CpG annotations showing various transcripts from the UCSC database (increasing gray bar height corresponds to introns, promoters, and exons, respectively). FIG. 9A shows a heatmap of CD8A. FIG. 9B shows a heatmap of CD4. FIG. 9C shows a heatmap of TIGIT. FIG. 9D shows a heatmap of LAG3. FIG. 9E shows a heatmap of CCR7. FIG. 9F shows a heatmap of SELL. FIG. 9G shows a heatmap of TNFRSF9. FIG. 9H shows a heatmap of CTLA4. FIG. 9I shows a heatmap of CXCR3. FIG. 9J shows a heatmap of SL AMF8. FIG. 9K shows a heatmap of CD69. FIG. 9L shows a heatmap of FOXP3. FIG. 9M shows a heatmap of EOMES. FIG. 9N shows a heatmap of TBX21. FIG. 9O shows a heatmap of UZMB. FIG. 9P shows a heatmap of CD19. FIG. 9Q shows a heatmap of KLF4. FIG. 9R shows a heatmap of MYC. FIG. 9S shows a heatmap of SOX2. FIG. 9T shows a heatmap of IL2. FIG. 9U shows a heatmap of IFNG. FIG. 9V shows a heatmap of IL2RG. FIG. 9W shows a heatmap of MKI67. FIG. 9X shows a heatmap of CD101. FIG. 9Y shows a heatmap of IL7R. FIG. 9Z shows a heatmap of CD30. FIG. 9AA shows a heatmap of CD3E. FIG. 9BB shows a heatmap of CD27. FIG. 9CC shows a heatmap of CD28. FIG. 9DD shows a heatmap of IL7R. FIG. 9EE shows a heatmap of IL2RB, FIG. 9FF shows a heatmap of CXCR1. FIG. 9GG shows a heatmap of CDCR4. FIG. 9HH shows a heatmap of BCL6. Dark gray indicates methylated state. Light gray indicates unmethylated state.

FIG. 10 shows a histogram of the optimal number of clusters based on the Gap Statistic for >144,000 Hg38 genes.

FIGS. 11A-11E shows histograms of the optimal number of clusters per chromosome based on the Gap Statistic for >14,0000 Hg38 genes. FIG. 11A shows from top to bottom histograms for chromosome 1, chromosome 14, chromosome 19, chromosome 3, and chromosome 8. FIG. 11B shows from top to bottom histograms for chromosome 10, chromosome 15, chromosome 2, chromosome 4, and chromosome 9. FIG. 11C shows from top to bottom histograms for chromosome 11, chromosome 16, chromosome 20, chromosome 5, and chromosome X. FIG. 11D shows from top to bottom histograms for chromosome 12, chromosome 17, chromosome 21, and chromosome 6. FIG. 11E shows from top to bottom histograms for chromosome 13, chromosome 18, chromosome 22, and chromosome 7.

FIGS. 12A-12Z and FIGS. 12AA-12II illustrate heatmaps of various genes located on the X chromosome showing optimal number of clusters based on the Gap Statistic. Row annotation (gray) are CpG annotations showing various transcripts from the UCSC database (increasing gray bar height corresponds to introns, promoters, and exons, respectively). FIG. 12A shows a heatmap of FOLA2. FIG. 12B shows a heatmap of EMD. FIG. 12C shows a heatmap of PGRMC1. FIG. 12D shows a heatmap of RPL10. FIG. 12E shows a heatmap of EOLA1 FIG. 12F shows a heatmap of HTATSF1. FIG. 12G shows a heatmap of NDUFB11 FIG. 12H shows a heatmap of CCNQ gene. FIG. 12I shows a heatmap of IKBKG. FIG. 12J shows a heatmap of SLC25A5. FIG. 12K shows a heatmap of TMEM185A. FIG. 12L shows a heatmap of ZBTB33. FIG. 12M shows a heatmap of AMER FIG. 12N shows a heatmap of DYNLT3. FIG. 12O shows a heatmap of PRPS1. FIG. 12P shows a heatmap of ZNF449. FIG. 12Q shows a heatmap of BCAP31. FIG. 12R shows a heatmap of ZNF711 FIG. 12S shows a heatmap of NALF2. FIG. 12I shows a heatmap of MORF4L2. FIG. 12U shows a heatmap of UBL4A. FIG. 12V shows a heatmap of ZNF41. FIG. 12W shows a heatmap of ARX. FIG. 12X shows a heatmap of FAM199X. FIG. 12Y shows a heatmap of RAP2C. FIG. 12Z shows a heatmap of F8A2. FIG. 12AA shows a heatmap of MCTS1. FIG. 12BB shows a heatmap of MED12. FIG. 12CC shows a heatmap of PRDX4. FIG. 12DD shows a heatmap of PRPS2, FIG. 12EE shows a heatmap of ERCC6L. FIG. 12FF shows a heatmap of LONRF3. FIG. 12GG shows a heatmap of SOWAHD. FIG. 12HH shows a heatmap of SYP. FIG. 12II shows a heatmap of TCEAL3. Dark gray indicates methylated state. Light gray indicates unmethylated state.

FIG. 13 shows a heatmap and plot of calculated information gain for the LAG3 gene. Higher values of information gain indicate those CpGs are more important in defining the clusters. Dark gray indicates methylated state. Light gray indicates unmethylated state.

FIG. 14 shows a heatmap and plot of calculated information gain for the MYC gene. Higher values of information gain indicate those CpGs are more important in defining the clusters. Dark gray indicates methylated state. Light gray indicates unmethylated state.

FIG. 15 depicts an example of sorting CD8+ T cells into naïve, central memory (CM), effector (Eff), and effector memory (EM) populations.

FIGS. 16A-16D depict exemplary epigenetic heatmaps generated of the GZMK gene in accordance with some embodiment. FIG. 16A depicts an exemplary epigenetic heatmap of the GZMK gene constructed from methylorne sequencing of naïve CD8+ T-cells. FIG. 16B depicts an exemplary epigenetic map of the GZMK gene constructed from methylome sequencing of central memory CD8+ T-cells. FIG. 16C depicts an exemplary epigenetic heatmap of the GZMK gene constructed from methylome sequencing of effector CD8+ T-cells. FIG. 16D depicts an exemplary epigenetic heatmap of the GZMK gene constructed from methylome sequencing of effector memory CD8+ T-cells.

FIGS. 17A-17D depict exemplary epigenetic heatmaps generated of the SELL gene in accordance with some embodiment. FIG. 17A depicts an example epigenetic heatmap of the SELL gene constructed from methylome sequencing of naïve CD8+ T-cells. FIG. 17B depicts an exemplary epigenetic map of the SELL gene constructed from methylome sequencing of central memory CD8+ T-cells. FIG. 17C depicts an exemplary epigenetic heatmap of the SELL gene constructed from methylome sequencing of effector CD8+ T-cells. FIG. 17D depicts an exemplary epigenetic heatmap of the SELL gene constructed from methylome sequencing of effector memory CD8+ T-cells.

FIGS. 18A-18D depict exemplary epigenetic heatmaps generated of the CD27 gene in accordance with some embodiment. FIG. 18A depicts an exemplary epigenetic map of the CD27 gene constructed from methylome sequencing of naïve CD8+ T-cells. FIG. 18B depicts an exemplary epigenetic heatmap of the CD27 gene constructed from methylome sequencing of central memory CD8+ T-cells. FIG. 18C depicts an exemplary epigenetic heatmap of the CD27 gene constructed from methylome sequencing of effector CD8+ T-cells. FIG. 18D depicts an exemplary epigenetic heatmap of the CD27 gene constructed from methylome sequencing of effector memory CD8+ T-cells.

FIG. 19 shows an epigenetic map of chromosome 1 (positions 55,037,760-55,066,456), depicting the methylation patterns in the PCSK9 gene in different cell types.

FIG. 20 shows a diagram depicting an epigenetic modulator as described herein and a method of targeted methylation of a promoter region to silence gene expression as described herein.

FIG. 21 shows an exemplary method for iteratively selecting a modification for one or more epigenetic markers.

FIG. 22 shows an exemplary method for iteratively selecting a modification for one or more epigenetic markers.

FIG. 23 shows an exemplary method modifying epigenetic markers in a cell according to a target list to generate a modified cell, according to some embodiments.

FIGS. 24A-24C illustrate plasmid constructs of ExpON, ExpOF, and MCP-VPR. FIG. 24A show plasmid constructs of ExpOFF. FIG. 24B show plasmid constructs of ExpON. FIG. 24C show plasmid constructs of MCP-VPR.

FIG. 25 shows flow analysis for BFP reporter expression in cells transfected with ExpOFF plasmid and CD151 targeting sgRNA, CD81 targeting sgRNA, or non-targeting sgRNA control.

FIGS. 26A-26C illustrate flow analysis for CD81 and CD151 expression in cells transfected with ExpOFF plasmid and CD151 or CD81 targeting sgRNA. FIG. 26A shows flow analysis for CD81 or CD151 expression in cells after 12 days post transfection. FIG. 26B shows flow analysis for CD81 or CD-151 expression in cells after 24 days post transfection. FIG. 26C shows flow analysis for CD81 or CD151 expression in cells after 35 days post transfection.

FIG. 27 shows epigenetic maps of chromosome 11 (positions 831,698-834,439), depicting the methylation patterns in the CD151 gene of edited cells and of control cells. Dark gray indicates unmethylated state. Light gray indicates methylated state.

FIG. 28 shows epigenetic heatmaps of chromosome 11 (positions 831,698-834,439) generated for edited cells and control cells. Dark gray indicates unmethylated state. Light gray indicates methylated state.

FIG. 29 shows an epigenetic map depicting the methylation patterns of a region of chromosome 19 for edited cells and control cells. Dark gray indicates unmethylated state. Light gray indicates methylated state,

FIG. 30 shows an epigenetic map depicting the methylation patterns of a region of chromosome 12 for edited cells and control cells. Dark gray indicates unmethylated state. Light gray indicates methylated state.

FIG. 31 illustrates an exemplary method for generating a personalized differential cellular state profile.

### DETAILED DESCRIPTION

Directly editing the epigenome (e.g., by methylating, demethylating, acetylating, or deacetylating chromosomal target sites) provides a direct means of controlling cellular state. Methylation and demethylation techniques can be used to modify both DNA targets and histone targets, while acetylation and deacetylation techniques can be used to modify histone targets. The disclosure provides methods of analyzing empirical data and/or other available (e.g., publicly available) data sets to select target epigenetic sites and/or epigenetic modifications. The selection may be iterative, for example by modifying a cell according to the selected target and/or modification, identifying effects of the modification (e.g., multi-ohmic and/or functional effects), and selecting a new target and/or effectors based on the identified effects.

The disclosure provides a method of selecting a modification for one or more epigenetic markers. The method may include obtaining a target list comprising epigenetic markers (e.g., one or more CpG sites and/or one or more histones) and an associated modification (e.g., methylation, demethylation, acetylation, and/or deacetylation) for each epigenetic marker. The target list may include targets associated with a desired cellular state (for example, a biological age and/or a disease state). The method may include modifying at least a portion of the epigenetic markers in a cell according to the target list to generate a modified cell. The method may include profiling the modified cell to determine a cellular state profile for the modified cell. The method may include selecting, based on the cellular state profile for the modified cell, an updated target list comprising updated epigenetic markers and an associated modification for each updated epigenetic marker. The method may further include determining a differential between the cellular state profile and a desired cellular state profile. The updated target list may be based on this differential. A cell may be reprogrammed by editing the cell based on the updated target list.

The method may be performed iteratively. For example, the method may further include modifying at least a portion of the epigenetic markers from the updated target list in a second cell to generate a second modified cell. The second modified cell can then be profiled to determine a cellular state profile for the second modified cell. Based on the cellular state profile for the second modified cell, a second updated target list, comprising second updated epigenetic markers and an associated modification for each second updated epigenetic marker, may be selected. This process may be repeated any number of desired iterations (e.g., at least 2, at least 3, at least 4, or at least 5 iterations).

The method may be used to select and/or evaluate a plurality of epigenetic markers. For example, the target list may include 2 or more, 10 or more, 25 or more, 50 or more, 100 or more, 500 or more, or 1000 or more epigenetic markers. The method may also be used to simultaneously modify a plurality of epigenetic markers in the cell according to the target list. For example, 2 or more, 10 or more, 25 or more, 50 or more, 100 or more, 500 or more, or 1000 or more epigenetic markers may be simultaneously modified in the cell.

The method may include, for example, predicting one or more (e.g., a plurality of) epigenetic modifications (e.g., a target site and/or target-site associated effectors). The method may include modifying a cell according to the one or more predicted epigenetic modifications. The method may further include profiling the cellular state of the cell to generate a cellular profile. The generated cellular profile may then be used as an input to predict one or more new epigenetic modifications.

The methods described herein allow for the determination of an epigenetic profile indicative of a single cell in a population of cells (e.g., a mixture of different cells). This process may be performed as a high-throughput process that allows the epigenetic profiles of many single cells in the population to be determined simultaneously. Nucleic acid molecules (e.g., DNA) can be obtained from the population of cells such that the isolated nucleic acid molecules contain a mixture of DNA from different cells. The DNA is sequenced to generate sequence reads, where the sequence reads include sequence information (i.e., nucleobase sequence) and epigenetic (e.g., base methylation status) information. Overlapping methylation information, along with the sequence information, between sequence reads can be assembled to generate long-range contigs. This allows for assembly of very long-range methylation information up to chromosome length. Further the unique contig that includes both sequencing information and methylation information is indicative of a single cell within the cell population.

A significant amount of heterogeneity in cellular reprogramming that can only be addressed by single cell level resolution. This heterogeneity is in terms of initial state and identity of cells (for example starting with a sorted population of CD8 T-cells, there is a mix of differentiation states). Each cell in the cell population may respond differently to reprogramming, ranging from not responding at all to quickly entering a pluripotent state.

Even though single-cell methylation sequencing has advanced significantly, the ability to profile dimethylation of a large number of single cells has been limited due to small amount of material from each cell and difficulty of translating single cell methylation assays to droplets and other physical compartments. The approach described herein addresses this limitation by long-read methylation sequencing of fragments that came from a pool of cells, which allows for reconstructing long-range methylomes indicative of individual cells, for example up to a chromosome in length.

The epigenetic method can include obtaining DNA molecules from a cell population; sequencing the DNA molecules to provide a plurality of sequence reads comprising a methylation status for a plurality of bases in each sequence read; and assembling a plurality of contigs based on the plurality of sequence reads using sequence information and methylation status for the sequence reads, wherein contigs having substantially the same sequence and different methylation profiles are identified as being associated with different cells in the cell population. The sequence reads may be long-range sequence reads, for example, about 1000 to about 100,000 bases in length. The sequencing reads are assembled using the sequencing information and the methylation status information, which generates a contig that can be up to an entire chromosome in length.

The long-range epigenetic profiling data is indicative of an individual cell in the cell population. This information is particularly useful for evaluating cells that are undergoing cellular reprogramming or that have been subject to cellular reprogramming. For example, the epigenetic profile of a cell in the cell population may be compared to a target epigenetic profile to determine a differential. This can be used to indicate how close a cellular reprogramming protocol is to obtaining the desired target epigenetic profile given the starting state of the cell.

### Terminology

Unless defined otherwise, all technical and scientific terms used have the meaning commonly understood by one of ordinary skill in the art. The following terms have the meanings given:

Singular forms “a,” “an,” “the,” and “said” include the plural forms as well, unless the context clearly indicates otherwise.

The terminology, “and/or.” used in a phrase such as “X and/or Y” includes both X and Y; X or Y; X (alone); and Y (alone).

The words “comprising,” “comprise,” “comprises,” “having.” “have,” “has,” “including,” “includes,” “include.” “containing,” “contains” and “contain” are inclusive or open-ended and do not exclude additional, unrecited elements or method steps. Aspects and embodiments of the invention described include “comprising,” “consisting of,” and “consisting essentially of” (and variants thereof) aspects and embodiments.

Use of terminology like “some embodiments,” “an embodiment,” “one embodiment,” “other embodiments,” “various embodiments,” “another embodiment,” “some cases” and the like with reference to a particular feature or characteristic described in connection with the embodiment or case, means that the feature is included in one or more embodiments, but not necessarily all embodiments. Similarly, reference to “a method,” “the method,” “one method” and the like with reference to a particular feature or characteristic described in connection with the method, means that the feature is included in one or more methods, but not necessarily all methods.

“About” and “approximately” refer to the usual error range for the respective value readily known to the skilled person in this technical field. Exemplary degrees of error are within 20 percent (%), typically, within 10%, and more typically, within 5% of a given value or range of values. Reference to “about” or “approximately” a value or parameter includes (and describes) embodiments directed to that value or parameter per se.

As used herein, the terms “align,” “aligned,” “alignment,” or “aligning” refer to the process of comparing a sequence read to a reference sequence or other sequence (e.g., another sequence read) and thereby determining whether the reference sequence or other sequence contains the sequence read sequence or a portion thereof. If the reference sequence contains the read, the read may be mapped to the reference sequence or, in certain embodiments, to a particular location in the reference sequence. In some cases, an alignment additionally indicates a location in the reference sequence where the sequence read maps to. For example, if the reference sequence is the whole human genome sequence, an alignment may indicate that a sequence read is present on chromosome 13, and may further indicate that the read is on a particular strand and/or site of chromosome 13. Alignment may occur between two or more sequence reads, for example to determine an overlapping sequence or sequence portion between the sequence reads. Alignment is typically implemented by a computer algorithm. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. The matching of a sequence read in aligning can be a 100% sequence match or less than 100% (non-perfect match).

“Determining,” “measuring,” “evaluating,” “assessing,” “assaying.” and “analyzing” refer to forms of measurement. include determining whether an element is present or not (for example, detection). can include quantitative, qualitative, or quantitative and qualitative determinations. Assessing can be relative or absolute. “Detecting the presence of” can include determining the amount of something present in addition to determining whether it is present or absent depending on the context.

“Cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include, for example, a hematologic cancer, a solid tumor, a soft tissue tumor, or a metastatic lesion. “Cancer” includes premalignant, as well as malignant cancers.

“Cell” refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an algal cell, a fungal cell an animal cell, a cell from an invertebrate animal (e.g. fruit fly, enidarian, echinoderm, nematode, etc.), a cell from a vertebrate animal (e.g., fish, amphibian, reptile, bird, mammal), a cell from a mammal (e.g., a pig, a cow, a goat, a sheep, a horse, a rodent, a rat, a mouse, a non-human primate, a human, etc.). A cell can be a somatic cell, for example, a skin cell, a nerve cell, a muscle cell, a blood cell, a muscle cell, a liver cell, a skin cell, an immune cell, a pancreatic cell, a nerve cell, a gastric cell, a cardiac cell, a gonad cell, or a fat cell, a bone cell (e.g., osteoblast, osteocyte, osteoclast, osteoprogenitor cell), a brain cell (e.g., neuron, astrocyte, glial cell), an optic cell, an olfactory cell, an auditory cell, or a kidney cell, or a germ cell, e.g., an oocyte, a sperm. In some embodiments, the cell may be an adult cell, e.g., adult somatic cell, a sperm, an oocyte. In some embodiments the somatic cell is an “adult somatic cell,” by which is meant a cell that is present in or obtained from an organism other than an embryo or a fetus or results from proliferation of such a cell in vitro. Unless otherwise indicated, the compositions and methods for rejuvenating a somatic cell can be performed both in vivo and in vitro, where in vivo is practiced when a somatic cell is present within a subject, and where in vitro is practiced using an isolated somatic cell maintained in culture. In some embodiments, the cell may be a stein cell, e.g., an embryonic stem cell, an adult stem cell, an induce pluripotent stem cell (iPSC). Induced pluripotent stein cells can be derived, for example, from adult somatic cells such as skin or blood cells. In some embodiments, the stem cell may be a totipotent stein cell, a pluripotent stem cell, a multipotent stem cell, or an unipotent stein cell. Certain other cell-related terminology is defined as follows:

(A) “Allogeneic cell” refers to a cell obtained from an individual who is not the intended recipient of the cell as a therapy (the cell is allogeneic to the subject). Allogeneic cells of the disclosure may be selected from immunologically compatible donors with respect to the subject of the methods of the disclosure, Allogeneic cells of the disclosure may be modified to produce “universal” allogeneic cells, suitable for administration to any subject without unintended immunogenicity. Allogeneic cells of the disclosure include, but are not limited to, hematopoietic cells and stem cells, such as hematopoietic stem cells.

(B) “Autologous cell” refers to a cell obtained from the same individual to whom it may be administered as a therapy (the cell is autologous to the subject). Autologous cells of the disclosure include, but are not limited to, hematopoietic cells and stein cells, such as hematopoietic stem cells.

(C) “Cell therapy” refers to the delivery of a cell or cells into a recipient for therapeutic purposes. Cells described herein may be used in compositions and methods of cell therapy.

(D) “Hematopoietic cell” may refer to a cell that arises from a hematopoietic stein cell. This includes, but is not limited to, myeloid progenitor cells, lymphoid progenitor cells, megakaryocytes, erythrocytes, mast cells, myeloblasts, basophils, neutrophils, eosinophils, macrophages, thrombocytes, monocytes, natural killer cells, T lymphocytes, B lymphocytes and plasma cells.

(E) “Induced pluripotent stem cell” (IPS or iPSC) refer to a pluripotent stein cell that can be generated directly from a somatic cell. This includes, but is not limited to, specialized cells such as skin or blood cells derived from an adult.

(F) “Mesenchymal cell” refers to a cell that is derived from a mesenchymal tissue. In some cases, cells of the disclosure may be mesenchymal cells.

(G) “Mesenchymal stromal cell” (MSC) may refer to a spindle shaped plastic-adherent cell isolated from bone marrow, adipose, and other tissue sources, with multi potent differentiation capacity in vitro. For example, a mnesenchymnal stromal cell can differentiate into osteoblasts (bone cells), chondrocytes (cartilage cells), myocytes (muscle cells), and adipocytes (fat cells which give rise to marrow adipose tissue). The term mesenchymal stromal cell is suggested in the scientific literature to replace the term “mesenchymal stem cell.” In some cases, cells of the disclosure may be mesenchymal stromal cells.

(H) “Mesenchyme” refers to a type of animal tissue included of loose cells embedded in a mesh off proteins and fluid, i.e., the extracellular matrix. Mesenchymne directly gives rise to most of the body's connective tissues including bones, cartilage, lymphatic system, and circulatory system.

(I) “Multipotent” refer to a cell that can develop into more than one cell type but is more limited than a pluripotent cell. For example, adult stem cells and cord blood stem cells may be considered as multipotent.

(J) “Pluripotent stem cell” (PSC) may refer to a cell that can maintain an undifferentiated state indefinitely and can differentiate into most, if not all cells of the body.

(K) “Stem cell” refer to an undifferentiated or partially differentiated cell that can differentiate into various types of cells and proliferate indefinitely to produce more of the same stem cell.

(L) “T-lymphocyte” or T-cell” refer to a hematopoietic cell that normally develops in the thymus. T-lymphocytes or T-cells include, but are not limited to, natural killer T cells, regulatory T cells, helper T cells, cytotoxic T cells, memory T cells, gamma delta T cells, and mucosal invariant T cells.

(M) “Transfect,” “transform” and “transduce” refer to a process by which exogenous nucleic acid is transferred or introduced into a cell or a host cell. A “transfected” or “transformed” or “transduced” cell is one which has been transfected, transformed, or transduced with exogenous nucleic acid or progeny of the cell.

The term “contacting a cell” with a substance, refers to contacting the cell with said substance internally or externally, and includes expressing said substance in said cell, unless context clearly indicates otherwise. For example, contacting a cell with a culture medium includes culturing said cell in said culture medium. Contacting a cell with a cellular reprogramming factor can include incubating or culturing said cell in a medium containing said cellular reprogramming factor, or inducing expression of said cellular reprogramming factor within said cell (for example, if the cellular reprogramming factor is a biologic cellular reprogramming factor).

A “cellular reprogramming factor” refers to any substance (e.g., salt, small-molecule compound, or biologic) that directly or indirectly regulates an epigenetic profile of a cell. A cellular reprogramming factor may modify the epigenetic profile of a cell directly by, for example, directly methylating, demethylating, acetylating, or deacetylating a nucleobase or histone. A cellular reprogramming factor may indirectly modify the epigenetic profile, for example, by causing expression of another cellular reprogramming factor that directly or indirectly modifies the epigenetic profile, or recruiting (e.g., by direct or indirect binding) another cellular reprogramming factor that directly or indirectly modifies the epigenetic profile.

“Complementary” and “complementarity” refer to the association of double-stranded nucleic acids by base pairing through specific hydrogen bonds. The base parling may be standard Watson-Crick base pairing (e.g. 5′-A G T C-3′ pairs with the complementary sequence 3-T C A G-5′) or other non-traditional type. Complementarity is typically measured with respect to a duplex region and thus, excludes overhangs, for example. Complementarity between two strands of the duplex region may be partial and expressed as a percentage (e.g., 80%), if only some (e.g., 80%) of the bases are complementary.

“CpG Island” refers to a region with a high frequency of CpG sites. The region is at least 200 bp, with a GC percentage greater than 50%, and an observed-to-expected CpG ratio greater than 60%.

“Diagnose” and “diagnosis” refer to the identification or classification of a molecular or pathological state, disease, or condition (e.g., cancer). For example, “diagnosis” may refer to identification of a particular type of cancer. “Diagnosis” may also refer to the classification or staging of a particular subtype of cancer, for instance, by histopathological criteria, or by molecular features (e.g., a subtype characterized by expression of one or a combination of biomarkers (e.g., genes or proteins encoded by said genes)).

“Domain” refers to a section or portion of a polypeptide or a nucleic acid sequence encoding the section or the portion of the polypeptide that contributes to a specified function to the polypeptide. A domain may comprise a contiguous region or more than one distinct non-contiguous regions of a polypeptide.

“Edit” and “editing” with reference to a nucleic acid refers to any change in nucleic acid, including insertion, deletion, and correction. “Editing” can also refer to any epigenetic changes or epigenetic editing. In some cases, “epigenetic editing” refers to the selective and reversible modification of DNA (e.g., methylation, demethylation) and histories (methylation, demethylation, acetylation, deacetylation). The changes can be in a genome of a cell. “Insertion,” “deletion,” and “correction” have the following meanings:
(A) “Insertion” refers to an addition of one or more nucleotides in a DNA sequence. Insertions can range from small insertions of a few nucleotides to insertions of large segments such as a cDNA or a gene.
(B) “Deletion” refers to a loss or removal of one or more nucleotides in a DNA sequence or a loss or removal of the function of a gene. In some cases, a deletion can include, for example, a loss of a nucleotide, a few nucleotides, an exon, an intron, a gene segment, or the entire sequence of a gene. Deletion of a gene may include any deletion sufficient result in the elimination or reduction of the function or expression of the gene or its gene product.
(C) “Correction” refers to a change of one or more nucleotides of a genome in a cell, whether by insertion, deletion, or substitution.

Editing may also result in a gene knock-in, knock-out or knock-down, each defined as follows:
(A) “Knock-in” refers to an addition of a DNA sequence, or fragment thereof into a genome.
(B) “Knockout” refers to the elimination of a gene or the expression of a gene.
(C) “Knock-down” refers to reduction in the expression of a gene or its gene product(s).

“Epigenetic modulator” and “epigenetic effector” refer to a polypeptide engineered to bind a specific target sequence in chromosomal DNA and modify the DNA or protein(s) associated with DNA at or near the target sequence and modify the target sequence. An epigenetic modulator may, in some cases, include a nucleic acid binding moiety and one or more effector moieties. “Effector moiety” refers to a domain that can alter the expression of a target gene when localized to an appropriate site in the nucleus of a cell, e.g., in a target nucleotide sequence.

“Enhancer” as used herein refers to distal genetic elements that positively regulate gene expression in an orientation-independent manner in ectopic heterologous gain-of-function expression. Enhancer sequences bind transcription factors and are correlated with specific chromatin features including but not limited to reduced DNA methylation, characteristic histone modifications, heightened chromatin accessibility, long-range promoter interactions, and bidirectional transcription.

“Epigenetic map” as used herein refers to any modes of representation of epigenetic states across a plurality of different regions, e.g., coding sequences, intergenic spacers, regulatory regions, e.g., promoters, etc., of the entire genome, a portion of the genome or near or around or within a particular gene or genes. An “epigenetic marker” refers to the collection of a locus and epigenetic status (e.g., methylated or non-methylated) of a nucleic acid residue in an epigenome. A “loss” of an “epigenetic marker” refers to a change of the epigenetic status of the epigenetic marker relative to a comparator or control.

“Gene” refers to a combination of polynucleotide elements, that when operatively linked in either a native or recombinant manner, provide some product or function. “Gene” is to be interpreted broadly and can encompass mRNA, cDNA, cRNA and genomic DNA forms of a gene. In some uses, “gene” encompasses the transcribed sequences, including 5′ and 3′ untranslated regions (5-UTR and 3′-UTR), exons and introns. In some genes, the transcribed region will contain “open reading frames” that encode polypeptides. In some cases, a “gene” comprises only the coding sequences (e.g., an “open reading frame” or “coding region”) necessary for encoding a polypeptide. In some cases, a “gene” may not encode a polypeptide, for example, ribosomal RNA genes (rRNA) and transfer RNA (tRNA) genes. In some cases, a “gene” may include not only the transcribed sequences, but in addition, also includes non-transcribed regions including upstream and downstream regulatory regions, enhancers, and promoters.

“Guide RNA,” “g RNA,” “single guide RNA,” and “sgRNA” refer to any RNA molecule (or a group of RNA molecules collectively) that facilitates binding of a polypeptide, such as a Cas protein, to a specific location of a target nucleic acid. A single guide RNA (sgRNA) can comprise a crRNA and tracrRNA that are fused together. A guide RNA (gRNA) can comprise a crRNA segment and/or a tracrRNA segment. Exemplary guide RNAs include, but are not limited to, crRNAs, pre-crRNAs (e.g., DR-spacer-DR), and mature crRNAs (e.g., mature JDR-spacer, mature DR-spacer-mature JDR). “Guide RNA” also encompasses an RNA molecule or suitable group of molecular segments that binds a Cas protein other than Cas9 (e.g., Cpf1 protein) and that possesses a guide sequence within the single or segmented strand of NA comprising the functions of a guide RNA which include Cas protein binding to form a gRNA:Cas protein complex capable of binding, nicking and/or cleaving a complementary target sequence in a target polynucleotide.

“Homolog” refers to a gene or a protein that is related to another gene or protein by a common ancestral DNA sequence and is functionally similar. Homologous proteins may, but need not be structurally related or are only partially structurally related. “Ortholog” refers to a gene or protein that is related to another gene or protein by a speciation event. Orthologous proteins may in some cases be structurally related or only partially structurally related. In some cases, an ortholog may retain the same function as the gene or protein to which they are orthologous. Non-limiting examples of Cas9 orthologs include: Akkermansia muciniphila Cas9 (AmCas9), Bifidobacterium longum Cas9 (BlCas9), Campylobacter jejuni Cas9 (CjCas9), Francisella novicida Cas9 (FnCas9), Geobacillus stearothernophilus Cas9 (GeoCas9), Legionella pneumophila Cas9 (LpCas9), Neisseria lactamica Cas9 (NlCas9), Neisseria meningitidis Cas9 (NmCas9), Oscillospira luneus Cas9 (OlCas9), Staphylococcus aureus Cas9 (SaCas9), Streptococcus pyogenes Cas9 (SpCas9), Streptococcus thernophilus CRISPR1 Cas9 (St1Cas9), Streptococcus themophilus CRISPR3 Cas9 (St3Cas9). Homologs and orthologs may be identified by homology modeling (e.g., see Filipek, S. (2023). Homology modeling: Methods and protocols. Humana Press.).

“Individual,” “patient,” and “subject” refer to any single subject. e.g., mammal (including such non-human animals as, for example, dogs, cats, horses, rabbits, zoo animals, cows, pigs, sheep, and non-human primates) for which treatment is desired. In particular embodiments, the patient is a human.

“Methylate” and “methylating” refer to (i) the addition one or more methyl groups to one or more cysteine residues, or (ii) the replacement of one or more unmethylated cysteine residues with one or more methylated cysteine residues, or (iii) the addition of one or more methyl to one or more sites to one or more histones. “Demethylate” and “demethylating” refer to (i) the removal of one or more methyl groups from one or more cysteine residues, or (ii) the replacement of one or more methylated cysteine residues with one or more unmethylated cysteine residues, or (iii) the removal of one or more methyl residues from one or more sites on one or more histones.

“Modifying,” “modification,” “modulate” and “modulating” refer to a change in the structure, expression levels or activity of a gene or polypeptide as detected by standard art known methods such as those described herein. a modification (e.g., increase or decrease) includes a 10% change in expression levels, a 25% change, a 40% change, and a 50% or greater change in expression levels.

A “nucleobase sequence” refers to a nucleic acid sequence without respect to a methylation status of the nucleobase. Thus, for example, a nucleic acid molecule having a methylated cytosine is considered to have the same nucleobase sequence as an equivalent nucleic acid molecule having an unmethylated cytosine at the same position.

The term “overlapping” in the context of overlapping sequence reads refers to two or more sequence reads each having a portion with the same nucleobase sequence.

“Polynucleotide,” “oligonucleotide,” “nucleic acid,” and “nucleic acid sequence” are used interchangeably to refer to a polymeric form of nucleotides, such as deoxyribonucleotides, ribonucleotides, NS analogs thereof. Polynucleotides may be provided in single-, double-, or multi-stranded form in a linear, branched, or circular conformation. A polynucleotide can be exogenous (e.g., a sequence that is not native to the cell, or a chromosomal sequence whose native location in the genome of the cell is in a different chromosomal location) or endogenous (e.g., a chromosomal sequence that is native to the cell) to a cell. A polynucleotide can exist in a cell-free environment. A polynucleotide can be a gene or fragment thereof. A polynucleotide can be DNA. A polynucleotide can be RNA, e.g., an mRNA. A polynucleotide can comprise one or more analogs (e.g., altered backbone, sugar, or nucleobase). Non-limiting examples of modifications include addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-0-methyl nucleotides, locked nucleic acids (LNA), peptide nucleic acids (PNA), and morpholinos. If present, modifications to the nucleotide structure can be imparted before or after assembly of the polymer.

“Profile” refers to a set of one or more biological features determined from a sample. Exemplary features that may be included in a profile include, but are not limited to, epigenetic features (e.g., methylation and/or acetylation status of a CpG site or histone), nucleic acid sequence data, expression data, proteomics data, metabolomics data, results from a functional assay, cellular morphological characteristics, etc. “Cellular profile,” “epigenetic profile,” and “personalized differential cellular state profile” have the following meanings:

(D) “Cellular profile” refers to phenotypic and epigenetic state of a whole cell. “Cellular profile” also refers to the epigenetic characteristics of a cell's genome. Non-limiting examples of epigenetic characteristics include DNA methylation, DNA demethylation, histone methylation, histone demethylation, histone acetylation, histone deacetylation and combinations thereof.

(E) “Epigenetic profile” and “epigenome profile” refer to the epigenetic state of a whole genome. “Epigenetic profile” and “epigenome profile” also refer to epigenetic characteristics of genomic sequences in cells or tissues. Non-limiting examples of epigenetic characteristics include DNA methylation, DNA demethylation, histone methylation, histone demethylation, histone acetylation, histone deacetylation and combinations thereof.

(F) “Personalized differential cellular state profile” refers to the cellular profile of a cell compared to a healthy and/or young cell of similar type.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nln.nih.gov. A “genome” refers to the complete genetic information of an organism or virus, expressed in nucleic acid sequences. Exemplary reference sequences or reference genomes include the following assemblies: hg38 (human), hg19 (human), hg18 (human), hg17 (human), hg 16 (human), mm39 (mouse), mm10 (mouse), mm9 (mouse), mm8 (mouse), mm7 (mouse), mm6 (mouse). Other reference genomes and reference sequences are known in the art, include genomes from mammal, birds, fish, insects, fungi, bacteria, viruses, and archea. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In various embodiments, the reference sequence is a consensus sequence or other combination derived from multiple individuals. However, in certain applications, the reference sequence may be taken from a particular individual.

“Reprogram.” “transdifferentiate” and the like refer to a process that alters or reverses the differentiation state of a differentiated cell (e.g., a somatic cell). Reprogramming can encompass complete reversion of the differentiation state of a differentiated cell (e.g., a somatic cell) to a pluripotent state or a multipotent state. Reprogramming can encompass complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. Programming of a differentiated cell (e.g., a somatic cell) according; to the methods of the disclosure can cause a differentiated cell to assume a less differentiated state, or an undifferentiated state (e.g., an undifferentiated cell).

“Sample,” refers to a composition that is obtained or derived from a subject and/or individual of interest that contains or may contain a cellular and/or other molecular entity that is to be characterized and/or identified, for example, based on physical, biochemical, chemical, and/or physiological characteristics. Samples include, but are not limited to, tissue samples, primary or cultured cells or cell lines, cell supernatants, cell lysates, platelets, scrum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

“Sequence homology” and “sequence identity” refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. As a practical matter, whether any particular sequence can be at least 50%, 60%, 70%, 80%, 85%, 90%, 92%, 95%, 96%, 97%, 98% or 99% identical to any sequence described he (which can correspond with a particular nucleic acid sequence described herein), such particular polypeptide sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. For an amino acid sequence, in some cases, the sequence identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty 0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first ten residues at the N-terminus. The ten unpaired residues represent 10% of the sequence (number of residues at the N- and C-termini not matched/total number of residues in the query sequence) so 10% can be subtracted from the percent identity score calculated by the FASTDB program. If the remaining ninety residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

“Subject,” “host,” and “individual,” are as used interchangeably to refer to animals, typically mammalian animals. Any suitable mammal can be treated by a method or composition described herein, Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some cases, a mammal is a human. A mammal may be any age or at any stage of development (e.g., an adult, teen, child, infant, or a mammal in utero). A mammal may be male or female. A mammal can be a pregnant female. In some case, a subject may be a human. In some cases, a human may be more than about: 1 day to about 10 months old, from about 9 months to about 24 months old, from about 1 year to about 8 years old, from about 5 years to about 25 years old, from about 20 years to about 50 years old, from about 1 year old to about 130 wears old or from about 30 years to about 100 years old. Humans can be more than about: 1, 2, 5, 10, 2030, 40, 50, 60, 70, 80, 100, 110, or 120 years of age. Humans can be less than about: 1, 2, 5, 10, 20, 30, 40, 50, 60, 70, 80, 90, 100, 110, 120 or 130 years of age.

Contigs or sequence reads having “substantially the same” nucleobase sequence refers to contigs or sequence reads having 95% or higher sequence identity. As used herein, the term “percent sequence identity” refers to the degree of identity between any given query sequence and a subject sequence. A query nucleobase sequence is aligned to one or more subject nucleobase sequences using the computer program ClustalW (version 1.83, default parameters), which allows alignments of nucleic acid or protein sequences to be carried out across their entire length (global alignment). Chenna et al. (2003) Nucleic Acids Res. 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage: number of top diagonals: 4; and gap penalty: 5. For an alignment of multiple nucleic acid sequences, the following parameters are used: gap opening penalty: 10.0; gap extension penalty: 5.0; and weight transitions: yes. The output is a sequence alignment that reflects the relationship between sequences. ClustalW can be inn, for example, at the Baylor College of Medicine Search Launcher website and at the European Bioinformatics Institute website on the World Wide Web.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements, Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

Certain figures illustrate processes according to various examples. These exemplary processes may be performed, for example, using one or more electronic devices implementing a software platform. In some examples, one or more of the exemplary processes are performed using a client-server system, and the blocks of the illustrated processes may be divided up in any manner between the server and a client device. In other examples, the blocks of the exemplary processes are divided up between the server and multiple client devices. Thus, while portions of the exemplary processes are described herein as being performed by particular devices of a client-server system, it will be appreciated that the processes are not so limited. In other examples, one or more of the exemplary processes are performed using only a client device (e.g., user device) or only one or more client devices. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

All publications, patents, and patent applications mentioned in this specification are herein incorporated by reference in their entirety and for all purposes to the same extent as if each individual publication, patent, or patent application was specifically and individually indicated to be incorporated by reference in its entirety. In the event of a conflict between a term herein and a term in an incorporated reference, the term herein controls.

### Epigenetic Profiling Methods

A method for determining an epigenetic profile indicative of a single cell in a cell population can include obtaining DNA molecules from a cell population; sequencing the DNA molecules to provide a plurality of sequence reads comprising a methylation status for a plurality of bases in each sequence read; and assembling a plurality of contigs based on the plurality of sequence reads using sequence information (i.e., nucleobase sequence) and methylation status for the sequence reads, wherein contigs having substantially the same sequence and different methylation profiles are identified as being associated with different cells in the cell population.

The cell population may be a cultured population, or a population of cells obtained from a sample (e.g., a tissue sample) from an individual. In some embodiments, the cells are obtained from a cell line. Exemplary cells in the population of cells can include fibroblasts, keratinocytes, peripheral mononuclear blood cells, hepatocytes, neural cells, blood cells, immune cells, lung cells, pancreatic beta cells, cardionyocytes, oligodendrocytes, or epithelial cells. In some embodiments, the cells in the population of cells include pancreatic beta cells. In some embodiments, the cells in the population of cells include pancreatic alpha cells.

Nucleic acid molecules may be extracted from tissue samples, biopsy samples, blood samples, or other bodily fluid samples using any of a variety of techniques known to those of skill in the art (see, e.g., Tan, et al., DNA, RNA, and Protein Extraction. The Past and The Present, J. Biomed. Biotech. Vol. 2009, no. 574398 (2009). A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing; through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits, Examples include, but are not limited to, the QIAmp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

In some instances, the cell population may be derived from a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (mbion, Cat. No. AM1975, September 2008): the Maxwell® 16 FFPE Plus LEV DNA Purification Kit Technical Manual (Promega Literature #TM349, February 2011); the E.Z.N.A.® & FFPE DNA Kit Handbook (OMEGA bio-tek, Noreross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell®16 FFPE Plus LEV DNA Purification Kit is used with the Maxwell® 16 Instrument for purification of genomic DNA from 1 to 10 μm sections of FFPE tissue. DNA may be purified using silica-clad paramagnetic particles (PMPs) and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

After isolation, the nucleic acids may be dissolved in a slightly alkaline buffer, e.g., Tris-EDTA (TE) buffer, or in ultra-pure water. In some instances, the isolated nucleic acids (e.g. genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art, Methods for DNA shearing are described in Example 4 in International Patent Application Publication No. WO 2012/092426. In some instances, alternatives to DNA shearing methods can be used to avoid a ligation step during library preparation.

Sequencing of the DNA molecules obtained from the cell population can provide a plurality of sequence reads that each include a methylation status for the plurality of bases in each sequence read. Long-range sequencing technologies may be used, for example to obtain sequence reads that are over 1000 bases in length, for example about 1000 to about 100,000 bases in length. In some implementations, the sequence reads are about 1000 to about 5000 bases in length, about 5000 to about 10,000 bases in length, about 10,000 to about 20,000 bases in length, about 20,000 to about 50,000 bases in length, or about 50,000 to about 100,000 bases in length.

The sequencing method may provide a direct determination of methylation status for bases in the DNA molecules in addition to nucleobase sequence information. Direct determination of methylation status allows, for example, the sequencing method to directly distinguish a methylated base from a non-methylated base without converting the methylation status of a base. For example, the direct determination method can avoid the use of bisulfite treatment of the DNA molecules. The sequence read obtained through the sequencing method can include both nucleobase sequence and the methylation status (e.g., whether a particular base is methylated or unmethylated) of one or more bases in the sequence read.

Nanopore sequencing is an exemplary technique that may be used. Nanopore sequencing allows for the direct identification of nucleic acid base modification, including 5-methylcytosine (5mC), 5-hydroxymethylcytosine (5hmC), N6-methyladenine (6 mA), and bromodeoxyuridine (BrdU). See, for example, Oxford Nanopore Technologies, Benchmarking nanopore methylation analysis by comparison to publicly available bisulfite datasets, available at nanoporetech.com/resource-centre/benchmarking-nanopore-methylation-analysis-companson-publicly-available-bisulphite; Simpson et al., Detecting DNA cytosine methylation using nanopore sequencing, Nature Methods vol. 14, pp. 407-410 (2017); Rand et al., Mapping DNA methylation with high-throughput nanopore sequencing, Nature Methods, vol. 14, pp. 411-413 (2017); and Yuen et al., Systematic benchmarking of tools for CpG methylation detection from nanopore sequencing, Nature Communications, vol. 12, no. 3438 (2021).

In another example, direct determination of methylation status for bases in the DNA molecules can be based on polymerase kinetics. For example, HiFi (also known as 5-base HiFi or single-molecule, real-time (SMRT) sequencing) sequencing techniques may be used to determine methylation (e.g., 5mC, 5hmC, or 6 mA) status within in a sequence. HiFi sequencing provides two channels of information: fluorescence and kinetics. Utilizing both enables highly accurate reads (fluorescence) plus methylation status (kinetics) from a single library. HiFi sequencing observes a polymerase incorporating fluorescently labeled nucleotides complementary to a native DNA strand. The label identifies the base (A, C, G, T). Epigenetic modifications like 5mC impact polymerase kinetics. A convolutional neural network model may be used to process polymerase kinetics to determine methylation status of each epigenetic marker (e.g., CpG site) within the sequence read. See, for example. Flusberg et al., Direct detection of DNA methylation during single-molecule, real time sequencing, Nature Methods, vol. 7, pp. 461-465 (2010); Tse et al., Genome-wide detection of cytosine methylation by single molecule real-time sequencing, Proc. Nat'l Acad. Sci, vol. 118, no. 5, p. e2019768118 (2021); and Ni et al., DNA 5-methylcytosine detection and methylation phasing using PacBio circular consensus sequencing, bioRxiv 2022.02.26.482074 (2022). Similar to the above methods, determining sequencing and epigenetic status information for the sequence reads, the single-molecule, real-time sequencing method does not rely on bisulfite treatment of the DNA molecules.

The sequence reads comprising sequencing information and methylation status information for bases in the sequence reads may be assembled to provide contigs. That is, a plurality of contigs nay be assembled from the sequence reads using sequence information (i.e., the nucleobase sequence) and methylation status (i.e., the methylation status of one or more epigenetic markers, e.g., one or more CpG sites) for the sequence reads. Sequence reads having the same sequence information and methylation statuses within overlapping portions are joined together to form a contig, which can be identified as being associated with an individual cell within the cell population. As methylation statuses may vary within the same nucleobase sequence, a plurality of contigs may be assembled from the sequence reads, with the different contigs identified as being associated with different cells in the cell population.

To assemble the contigs, according to some embodiments, the sequence reads may be aligned (or mapped) to a reference sequence. The reference sequence depends on the species of the cells in the cell population. Thus, preferably, the cells in the cell population are from the same species or, in some embodiments, the same individual. Alignment of the sequence reads to the reference sequence is based on the sequence data. Optionally, at this stage, the methylation information may be ignored to align the sequence reads to the reference sequence. In some implementations, a reference sequence coordinate may be assigned to the sequence read based on the alignment. Once sequence reads are mapped to the reference sequence, the methylation information for overlapping sequence reads can be analyzed to determine whether two or more sequence reads should be joined to form a contig. That is, joined sequence reads used to form the same contig should include the same methylation statuses of epigenetic markers in the overlapping regions.

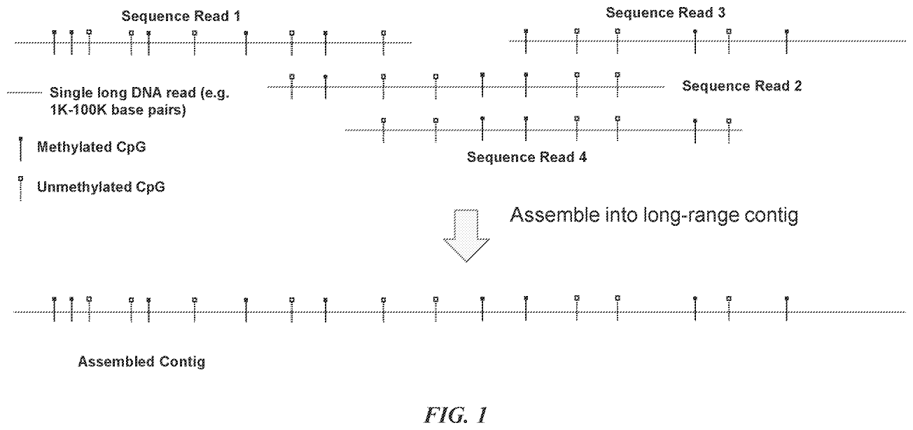

FIG. 1 shows an exemplary method for assembling sequence reads into a contig based on sequence information (i.e., nucleobase sequence) and methylation status for the sequence reads. In the illustrated example, the sequence of the 3′ end of Sequence Read 1 overlaps the sequence of the 5′ end of Sequence Read 2. The sequence of the 3′ end of Sequence Read 2 overlaps the sequence of the 5′ end of Sequence Read 3. Sequence Read 4 also overlaps portions of Sequence Read 1, Sequence Read 2, and Sequence Read 3. Because the sequences of these sequence reads overlap, the methylation status (i.e., epigenetic information) of the sequence reads is further analyzed before assembling the sequence reads into a contig. In particular, the status of epigenetic markers (e.g. CpG sites) is analyzed to determine a match between overlapping portions of the sequence reads. If the overlapping sequences between sequence reads also have the same methylation status for the epigenetic markers, then the sequencing reads are joined to form a contig. A match between the methylation status of the epigenetic markers of the sequence reads confirms assembly of the contig that includes both sequence information and epigenetic information.

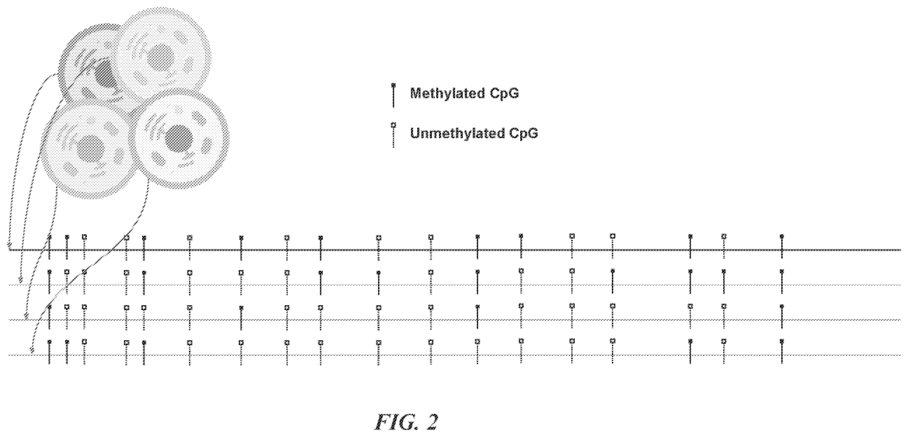

The contig assembly shown in FIG. 1 shows an example where all sequencing reads originate from the same single cell, and therefore form a single contig. When analyzing a cell population, however, there is diversity in the methylomes of the cells within the cell population. The genomes of the different cells in the cell population may be the same, but differences in the methylation status of various epigenetic markers may be different between cells within the population. Thus, the sequencing reads from the cell population may be assembled into a plurality of contigs, as indicated in FIG. 2, with each contig indicative of different single cells in the population.

The epigenetic profiling method may also be performed without the use of a reference sequence. Use of a reference sequence can make the process more computationally efficient, for example by assigning reference sequence coordinates to the sequence reads, which limits the number of sequence read comparisons for assembly of the contigs based on coordinate proximity. However, it is also possible to directly compare the sequence reads to identify overlapping portions of the sequence reads. Portions of the sequence reads having overlapping sequences may be further analyzed for methylation status (i.e., epigenetic information) within the overlapping portions to determine whether two or more sequence reads should be joined to form a contig. If the overlapping sequences between sequence reads also have the same methylation status for the epigenetic markers, then the sequencing reads are joined to form a contig. A match between the methylation status of the epigenetic markers of the sequence reads confirms assembly of the contig that includes both sequence information and epigenetic information.

Contigs having substantially the same nucleobase sequence and different methylation profiles are identified as being associated with different cells in the cell population. If, in contrast, the nucleobase sequences are entirely different, the methylation profiles may not be comparable. Further, the contigs may arise from different chromosomes from the same cell. If however, the nucleobase sequences of the contigs are substantially the same but the methylation profiles differ, it is more likely the contigs arose from the same chromosome in different cells. The nucleobase sequences of the contigs identified as coming from different cells need not be completely identical, as different cells may give rise to one or more variant or mutation profiles. In some embodiments, contigs identified as being associated with different cells in the cell population if the contigs have different methylation profiles and have 95% or greater, 96% or greater, 97% or greater, 98% or greater, 99% or greater, 99.5% or greater, or 99.9% greater sequence identity.

The assembled contigs may comprise a portion of a chromosome, a substantially complete chromosome, or a complete chromosome. In some embodiments, the assembled contigs comprise about 70% or more, about 75% or more, about 80% or more, about 85% or more, about 90% or more, or about 95% or more of a chromosome.

In some embodiments, the epigenetic profiling method comprises generating an epigenetic map that depicts methylation patterns from methylation sequence data from long-range sequence reads. In some cases, generating the epigenetic map comprises using machine learning methods. Generating the epigenetic map can comprise using unsupervised machine learning. In some cases, generating the epigenetic map comprises using clustering or cluster analysis. A method of unsupervised clustering of epigenetic maps can comprise selecting a region of interest. The method can further comprise extracting all fragments that span a genomic region (e.g., a gene), given a set of coordinates spanning the genomic region and the methylation status of any contained CpCs. The genomic region can be annotated as genes and/or promoter regions. A fragment can be a vector of binary values corresponding to CpGs with either methylated (1) or unmethylated (0) values. The method can further comprise computing a distance matrix comprising a distance measure between the fragments that span the genomic region. Non-limiting distance metrics for computing the distance between two binary-valued vectors include Hamming, Random Forest and Simple Matching. For example, a method using Simple Matching can evaluate the number of CpGs that match (e.g., both unmethylated or both methylated) and normalize to the total number of comparable CpGs in the region of interest.

The method can further comprise using the distance matrix to group various fragments to optimize an inter-cluster metric and an intra-cluster metric. For example, an inter-cluster metric can be optimized to minimize the inter-cluster average distance. As another example, an intra-cluster metric can be optimized to maximize the distance between the two closest residents of two separate clusters. Non-limiting examples of methods for clustering include hierarchical and k-means clustering. In some cases, a hierarchical clustering method can be an agglomerative, hierarchical clustering method with complete linkage.

The method can further comprise determining the optimal number of clusters. Determining the optimal number of clusters can comprise the Elbow Method, Silhouette, or the Gap Statistic. The method can comprise computing a figure of merit (FOM) while varying the number of clusters and selecting an optimal cluster number derived from the graph of the FOM vs. clusters (e.g., the elbow, maximum, etc.).

In some cases, the method comprises using the Gap Statistic. The Gap Statistic can comprise comparing the dispersion of inter-cluster distances to that obtained using a reference null distribution in which all samples are equidistant from one another. In some cases, there is only 1 cluster for the null hypothesis.

In some cases, the method comprises generating the reference null distribution. In some cases, for each CpG, a state (1 or 0) from the distribution of fragments that span that CpG is randomly sampled to generate the reference null distribution. In some cases, the resultant reference null data set ensures all features (i.e., CpGs) are independent of one another, thereby eliminating the dependency structure of the actual data. The method can further comprise generating a plurality of reference null distributions (e.g., by repeating the process). In some cases, the method comprises generating at least 5, at least 10, at least 20, at least 30, at least 40, at least 50, at least 60, at least 70, at least 80, at least 90, at least 100, at least 200, at least 300, at least 400, or at least 500 reference null distributions.

The method can further comprise calculating a dispersion FOM (log(Wk)) for each reference null distribution. The method can comprise repeating the calculation for varying cluster number (e.g., up to a maximum determined by the number of fragments for that gene). In some cases, the method further comprises comparing the mean of the reference distribution FOM for each cluster number to that obtained from the actual data and calculating the Gap Statistic. The method can further comprise using the standard error of the reference null FOM for each cluster number as a means to assess the impact of random sampling on a given FOM to another.

In some cases, the method comprises selecting the smallest cluster number (k) that satisfies Gap(k)=Gap(k+1)−3*SE(k+1). The method can a statistical approach to selecting the appropriate number of clusters based on the underlying data distributions.

Following determining the optimal number of clusters, the method can further comprise assigning fragments to the appropriate cluster. The method can further comprise adding annotations for each CpG.

In some cases, the method comprises generating a distribution of optimal number of clusters based on the Gap Statistic across at least 5,000, at least 6,000 at least 7,000, at least 8,000, at least 9,000, at least 10,000, at least 12,000, at least 14,000, at least 16,000, at least 18,000, at least 20,000, at least 25,000, at least 30,000, at least 35,000, at least 40,000, at least 50,000, at least 60,000, at least 70,000, at least 80,000, at least 80,000, or at least 100,000 genes.

The method of epigenetic profiling can enable the definition of epigenetic states at the gene level. In some cases, the method is used for multi-gene state profiling (e.g., whole genome profiling) by linking the states defined for one gene to those arising from a different gene. In some cases, the method uses fragments that span multiple genes to enable inter-genic correlations of epigenetic states. Alternatively, the method can use other data modalities such as single cell methylation profiling and/or gene expression to derive information about inter-genic state relationships.

In some cases, the method further comprises optimization methods to ensure that resultant clusters represent true epigenetic states. Optimization methods can include tightening the gap statistic selection criteria (increasing the number of SE(k+1)'s that Gap(k+1) must be from Gap(k)), placing an upper limit on the number of allowed epigenetic states per gene, denoising techniques to account for technical/biological noise, or incorporating various heuristics (e.g. weighting CpGs in promoter regions more heavily than introns in distance calculations, developing heuristics for accommodating known biological phenomenon such as X-inactivation).

In some cases, the method further comprises assessing the relative importance of GpGs to a given classification (e.g., cluster, experimental condition). This can, for example, can aid in differential analysis to identify favorable epigenetic editing target sites. The method can further comprise calculating an information gain for each CpG in a gene. Information gain can measure the gain in information (reduction in entropy) when partitioning a dataset on a given attribute (e.g., CpG methylation value). Information gain can be used in decision tree creation where it is used in a recursive fashion to select the order of attributes to partition on to maximize classification accuracy. Information gain can be calculated with the following equation:

Infonnation Gain=Entropy(T)−Entropy(T|a), where T is a random variable (e.g., epigenetic state) and a is an attribute (e.g., a specific CpG methylation status). Entropy(T|a) can be interpreted as the Expected value of the resulting entropy when the dataset is partitioned on attribute, a. Thus, given knowledge of the methylation of a CpG, how much information is gained regarding the underlying random variable (e.g., epigenetic state) can be calculated.

Entropy=−p*log 2(p)−(1−p)*log 2(1−p), where p is the probability of event in question (e.g., whether a given CpG is methylated or not).

In some cases, after the entropy of all clusters (i.e., all fragments) is first calculated, the weighted average of the entropy of each individual cluster of fragments is subtracted, thereby generating the information gain. Information gain of various genes can provide a method to quantitate the relative importance of a CpG methylation status on the underlying state classification.

The knowledge of the relative importance of various CpG to some classification (e.g., epigenetic state, experimental condition) can afford the ability to determine which CpG/genomic locations are most important in classification. This information can be used in applications including decision-tree based classification, targeted assays (e.g., use of panels vs, whole genome sequencing), or fundamental understanding of underlying biological processes (e.g., correlating regions of high information gain to differential expression of genes).

### Epigenetic Maps

The methods described herein utilize epigenetic maps of cells of different cellular states and cell types to identify unique methylation markers and patterns that may be contributors to a desired cellular state.

In some embodiments, an epigenetic map may be represented by coordinates compared to a reference genome. In some embodiments, an epigenetic map may be represented graphically. An epigenetic map may be physically displayed, e.g., on a computer monitor.

The mapping information can be obtained from the sequence reads to the region. In some embodiments, sequence read abundance, i.e., the number of times a particular sequence or nucleotide is observed in a collection of sequence reads may be calculated. In some embodiments, the epigenetic map depicting peak signals of sequence reads, e.g., as determined using peak-calling tools, can be generated. The resultant epigenetic map can provide an analysis of the chromatin in the region of interest. In some embodiments, the sequence reads are analyzed computationally to produce a number of numerical outputs that are mapped to a representation e.g., a graphical representation) of a region of interest.

In some instances, an epigenetic map may depict one or more of the following: chromatin accessibility along the region; DNA binding protein (e.g., transcription factor) occupancy for a site in the region, and/or chromatin states along the region. An epigenetic map may further represent the global occupancy of a binding site for the DNA binding protein by, e.g., aggregating data for one DNA binding protein over a plurality of sites to which that protein binds. In some instances, the map can be annotated with sequence information, and information about the sequence (e.g., the positions of introns, exons, transcriptional start sites, promoters, enhancers, etc.) so that the epigenetic information can be viewed in context with the annotation.

In some embodiments, an epigenetic map represents global changes in the methylation of across the entire genome of an organism, e.g., a human as well as changes in methylation of a plurality of different regions, e.g., coding sequences, intergenic spacers, regulatory regions, e.g., promoters, etc., of the entire genome, a portion of the genome or near or around or within a particular gene or genes. In some embodiments, an epigenetic map can represent the methylation level values of all CpG positions within entire genome of an organism, e.g., a human. In some embodiments, an epigenetic map can represent the methylation level values of all CpG positions within a plurality of different regions, e.g., coding sequences, intergenic spacers, regulatory regions, e.g., promoters, etc., of the entire genome, a portion of the genome or near or around or within a particular gene or genes.

In some embodiments, computationally implemented scripts or tools can be used to generate epigenetic/epigenomic maps. Exemplary scripts or tools that can be utilized include make_homer_ucse_file, which can create a .bedGraph file which allows for genome-wide pileups of fragment counts; and homer_bedgraph_to_bigwig which can convert the bedGraph file to a binary-compressed bigWig file, used by most genome browsers to visualize fragment coverage across the genome. The analysis can include generating a metric associated with particular elements of a gene. For example, such metrics can include accessibility over a promoter of an annotated gene, or over the coding region of an annotated gene. In some embodiments, annotation and generation of metric can be used for further downstream analysis, e.g., comparing epigenetic profiles, clustering and/or biological pathway analysis to produce a differential epigenetic map.

In some embodiments, an epigenetic map may be a differential epigenetic map. In some embodiments, a differential epigenetics map provides a representation of epigenetic modifications that have been made to across a plurality of different regions, e.g., coding sequences, intergenic spacers, regulatory regions, e.g., promoters, etc., of the entire genome, a portion of the genome or near or around or within a particular gene or genes compared to a reference. In some embodiments a differential epigenetics map provides a comparative representation of a first epigenetic map taken at a point in time and a second epigenetic map generated at another point of time to determine what changes have taken place in a specific time period. In some embodiments a differential epigenetics map provides a comparative representation of a first epigenetic map taken obtained before epigenetic modifications that have been made to across a plurality of different regions, e.g., coding sequences, intergenic spacers, regulatory regions, e.g., promoters, etc., of the entire genome, a portion of the genome or near or around or within a particular gene or genes and a second epigenetic map obtained after epigenetic modifications that have been made to across a plurality of different regions, e.g., coding sequences, intergenic spacers, regulatory regions, e.g., promoters, etc., of the entire genome, a portion of the genome or near or around or within a particular gene or genes. In some embodiments, a differential epigenetics map provides a representation of epigenetic differences between a plurality of different regions, e.g., coding sequences, intergenic spacers, regulatory regions, e.g., promoters, etc., of the entire genome, a portion of the genome or near or around or within a particular gene or genes located within a first cell and a plurality of different regions, e.g., coding sequences, intergenic spacers, regulatory regions, e.g., promoters, etc., of the entire genome, a portion of the genome or near or around or within a particular gene or genes located within a second cell. In some embodiments, the first cell and the second cell are of same type. In some embodiments, the first cell and the second cell are of different type. In some embodiments, the first cell and the second cell are of same age. In some embodiments, the first cell and the second cell are of different age, e.g., the first cell is an old cell, and the second cell is a young cell of the same type or vice versa. In some embodiments, the first cell and the second cell are in same cellular state. In some embodiments, the first cell and the second cell are in the different cellular state, e.g., the first cell is in a healthy state and the second cell is in a diseased state or vice versa.

In some embodiments, the epigenetic map can provide information regarding active regulatory regions and/or the transcription factors that are bound to the regulatory regions.

In some aspects, the methods described herein generate an epigenetic map that represents the epigenetic profile of a cell in a specific cellular state. The epigenetic map can present the epigenetic state (a methylation state, a 5′ hydroxymethylation state, a chromatin accessibility state, or a histone modification state) of a genomic site at a single-nucleotide resolution. In some cases, the epigenetic map represents the epigenetic profile of the whole genome of the cell in the specific cellular state. A cellular state can be a state of differentiation, a state of rejuvenation, a state of exhaustion, a state of memory, a biological age, a state of health, a state of disease, or a state of dysfunction. For example, a cellular state can comprise a level of stemness, a stem-like characteristic, or a memory characteristic. In another example, a cellular state can comprise a level of exhaustion, a level of differentiation, a disease-associated characteristic, a dysfunction-associated characteristic, or an age-associated characteristic.

In some embodiments, the methods described herein generate an epigenetic map that represents the epigenetic profile of a cell in a diseased state, an exhausted state or a dysfunctional state. In some embodiments, the epigenetic map represents the epigenetic profile of a cell in a healthy state, a rejuvenated state, or high-functioning state. In some embodiments, the epigenetic map represents the epigenetic profile of a cell in a young, more stemlike, or less differentiated cellular state. In some embodiments, the epigenetic map represents the epigenetic profile of a cell in an aged or more differentiated cellular state. For example, a cellular state may be an exhausted effector tumor infiltrating lymphocyte, a stemlike tumor infiltrating lymphocyte, a fibrotic state, a resident cell state, an induced pluripotent stem cell state, a target differentiated cell state, an alpha cell state, or a beta cell state.

In some aspects, the methods described herein generate an epigenetic map that represents the epigenetic profile of a cellular state of a specific cell or tissue type. A cell or tissue type may be defined by one or more characteristics, such as phenotypic properties (e.g., cell surface markers) or certain functional characteristics (e.g., ability to release cytokines). A cell type can also be classified by its tissue of origin (e.g., liver hepatocyte or blood granulocyte). For example, a cell may be a red blood cell, a white blood cell (e.g., a granulocyte or a lymphocyte), a liver hepatocyte, a cardiomyocyte, a pancreatic acinar cell, or an oligodendrocyte. In some embodiments, the methods described herein comprise profiling a cellular state of a lymphocyte (e.g., a natural killer cell, a T-cell, or a B-cell). In some cases, the lymphocyte is a T-cell. The T-cell may be a CD8+ T-cell, a CD4+ T-cell, or a regulatory T-cell.

In some aspects, generating an epigenetic map comprises methylome sequencing. Methylome sequencing may provide information about methylation states (e.g., methylated or unmethylated) of different sites in a gene or multiple genes. The methylome sequencing may be whole methylome sequencing and provide information about methylation states across the whole genome. Methylome sequencing may provide information about the methylation state at specific CpG sites or DNA methylations regions that regulate gene expression through transcriptional silencing of the corresponding gene. DNA methylation states may differ in different cell types or tissue types. DNA methylation states may differ based on state of differentiation, a state of rejuvenation, a state of exhaustion, a state of memory, a biological age, a state of health, a state of disease, or a state of dysfunction.

One or more epigenetic profiles described herein can be compared to identify a unique epigenetic marker or a unique epigenetic pattern (e.g., a unique methylation marker or a unique methylation pattern). In some cases, one or more epigenetic profiles described herein can be compared to identify a unique acetylation marker or a unique acetylation pattern. An epigenetic profile described herein can be used to identify a desired methylation or acetylation state at a specific genomic site. A differential between two or more epigenetic profiles described herein can identify a target site for modifying a cellular state to achieve a desired cellular state or to be closer to a desired cellular state. In some cases, detecting a differential in the two or more epigenetic profiles comprises comparing two or more epigenetic maps of the two or more epigenetic profiles. For example, a genomic site may be methylated in a first epigenetic profile and unmethylated in a second epigenetic profile. The differential at this genomic site can be detected by comparing the two epigenetic profiles. In some cases, a differential between two or more epigenetic profiles can be a differential in epigenetic state (e.g., methylation state) of a single nucleotide. In some cases, a differential between two or more epigenetic profiles can be a differential in epigenetic state (e.g., a methylation pattern) of a genomic region comprising at least 2, at least 4, at least 6, at least 8, at least 10, at least 20, at least 30, at least 40, at least 50, at least 60, at least 80, at least 100, at least 150, at least 200, at least 250, at least 300, at least 350, at least 400, at least 450, or at least 500 nucleotides. A list of one or more epigenetic target sites and associated modifications for each epigenetic target site may be selected computationally. For example, a machine-learning model trained to associate one or more modifications of an epigenetic marker to a desired cellular state (e.g., a desired biological age state or a desired disease state). Data used to train the model can include epigenetic profiling; data from a database (e.g., a publicly available database). Training data may additionally or alternatively include differential cellular state profiling data.

In some embodiments, epigenetic profiling is used in differential cellular state profiling. In some embodiments, the epigenetic profiling comprises an unsupervised clustering scheme described herein. In some embodiments, the unsupervised clustering scheme identifies epigenetic states on a whole genome scale. In some embodiments, the unsupervised clustering scheme identifies epigenetic states on a gene-level basis. In some embodiments, the unsupervised clustering scheme identifies epigenetic states on a whole genome and gene-level basis. In some embodiments, clustering scheme further comprises calculating the information gain for CpGs. In some cases, the information gained from a given classification (e.g., cluster) can provide information on the relative importance of a CpG methylation status on the underlying state classification (e.g., cluster).

### Cellular Identity Marker

In embodiments, the methods of epigenetic profiling described herein are used to identify a cellular identity marker. The epigenetic cellular identity marker can be correlated with the identity (i.e., cellular differentiation state) of cell. Loss of the epigenetic cellular identity markers may cause the cell to lose its cellular identity. See, for example, Basu et al., Epigenetic reprogramming o/cell identity: lessons rom development for regenerative medicine, Clinical Epigenetics, vol. 13, no. 144 (2021). The cellular identity of a cell can be the cellular differentiation state, for example, an immune cell (or particular type of immune cell), neural cell, epithelial cell, etc. In some cases, cell identity is dictated by the specific set of genes expressed and proteins produced in the cell that are activated by the epigenetic state of the cell to enable its unique function. In some cases, altering the epigenetic state of the epigenetic cellular identity markers causes a loss of cellular state identity.

In some embodiments, the epigenetic cellular identity marker is selected from a database. Such a database may be generated, for example, by comparing epigenetic profiles of different types of cells. The specific epigenetic sites across the genome of the different types of cells are compared and sites that are highly specific to a given tissue and cell are selected. For example, this could be in the form of a specific set of CpG sites in particular location in the genome that are unmethylated for cardiomyocytes but are methylated in all other tissues. Exemplary cellular identity markers are described in Moss et al., Comprehensive human cell-type methylation atlas reveals origins of circulating cell-free DNA in health and disease, Nat. Connmun., vol. 9, no. 5068 (2018); Loyfer et al., A human DINA methylation atlas reveals principles of cell type-specific methylation and identifies thousands of cell type-specific regulatory elements, Biorxiv 2022.01.24.477547 (2022); and Cui et al., A human tissue map of 5-hydroxymethylcytosines exhibits tissue specificity through gene and enhancer modulation, Nat. Commun., vol. 11, no. 6161 (2020),

### Cellular Reprogramming

The epigenetic profiling method described herein is particularly useful for analyzing cell populations that are undergoing cellular reprogramming or have been subject to cellular reprogramming. During cellular reprogramming, epigenetic markers of a cell are modified to alter, for example, the identity, disease state, or biological age of the cell. In a heterogenous population of cells, however, it is difficult to track the impact of a cellular reprogramming protocol on an individual cell. Because the epigenetic profiling methods described herein are able to determine the methylome of different cells in the cell population, they may be used to track changes to particular cells or determine the impact to particular cells undergoing cellular reprogramming.

A cell or population of cells can be partially reprogrammed by contacting the cell (or cells in a population) with one or more cellular reprogramming factors that modify one or more epigenetic markers. Optionally, the cellular reprogramming method may further include contacting the cell with a blocking reagent that specifically binds to the one or more epigenetic markers selected for preservation. The blocking reagent inhibits modification of the selected one or more epigenetic markers. For example, in some embodiments, it may be desirable to preserve specific epigenetic markers (e.g., one or more epigenetic markers correlated with cellular identity), and the blocking reagent may be used to limit the impact of the one or more cellular reprogramming factors. The cell may be simultaneously contacted with the blocking reagent and the one or more cellular reprogramming factors such that the blocking reagent inhibits the one or more modification enzymes from modifying the one or more epigenetic markers.

The one or more cellular reprogramming factors may include one or more one or more targeted cellular reprogramming factors that target one or more target epigenetic markers and/or may include one or more non-targeted cellular reprogramming factors (such as one or more, or all four, Yamanaka factors, or high potassium cell media). The method may further include culturing the cell after contacting the cell with the blocking reagent and the cellular reprogramming factors.

The method may further include selecting the one or more epigenetic markers and/or selecting the one or more target epigenetic markers (i.e., epigenetic marker targeted for modification), Selection may be, for example, based a known association between the epigenetic marker and a cellular identity, disease state, and/or biological age. As further described herein, selection may be based on the epigenetic markers of a desired cellular state profile.

Methods of partially reprogramming a cell may be performed in vivo (e.g., in a subject), ex vivo (e.g., outside of a subject), or in vitro (e.g., using; a cell line). For example, the one or more cellular reprogramming factors and/or blocking reagents may be administered to an individual. The cellular reprogramming factors and/or blocking reagents may be administered, for example, using a vector (such as a viral vector), which allows for expression of the cellular reprogramming factors and/or blocking reagents in the cell, which causes the partial reprogramming. In some implementations, the vector may be targeted to a particular cell type. In some embodiments, the method may be performed ex vivo, for example by obtaining a cell (or population of cells) from a subject. In some embodiments, the partially reprogrammed cell taking from the subject may then be readministered to the subject.

In some embodiments, the method may be used to partially reprogram an immune cell. In some embodiments, the method may be used to partially reprogram an immune cell ex vivo. In some embodiments, the method may be used to partially reprogram an immune cell for immunosenescence. In some embodiments, the method may be used to partially reprogram an immune cell for adoptive cell therapy. After partially reprogramming the cell, the partially reprogram cell may be, in some embodiments, administered to a subject, which may be the same subject or a different subject from which the original cell was obtained.

In some embodiments, the method may be used in partially reprogram a cell in vivo, Such partial reprogramming may be used to treat, for example, fibrosis in lung, liver, kidney, heart, or neurodegenerative disease, or type 2 diabetes. In some embodiments, the method may be used in partially reprogram a pancreatic beta cell in vivo.

The methods described herein include the use of one or more cellular reprogramming factors that modify one or more epigenic markers. The cellular reprogramming factors may be targeted or non-targeted (i.e., cause epigenetic modification at a plurality of different sites). In some embodiments, the one or more cellular reprogramming factors may include one or more transcription factors. For example, non-targeted cellular reprogramming transcription factors may include one or more Yamanaka factors (i.e., one or more of OCT4, SOX2, KIF4, and c-MYC). See, for example. Ocampo et al., In Vivo Amelioration of Age-Associated Hallmarks by Partial Reprogramming, Cell, vol. 167, pp. 1719-1733.e12 (2016); Gill et al., Multi-omic rejuvenation of human cells by maturation phase transient reprograming, Elife, vol. 11, e71624 (2022). Other cellular reprogramming factors can include small molecule and chemical cellular reprogramming factors. Exemplary small molecule cell reprogramming factors are described in Kim et al., Small-molecule-mediated reprogramming: a silver lining for regenerative medicine, Exp. Mol. Medicine, vol. 52, pp. 213-226 (2020); Bar-Nur et al., Small molecules facilitate rapid and synchronous iPSC generation, Nat. Methods, vol. 11, pp. 1170-1176 (2014). Exemplary chemical reagents are described in Guan et al., Chemical reprogramming of human somatic cells to pluripotent stem cells, Nature, vol. 605, pp. 325-331 (2022); Vodnala et al., T cell stemness and dysfunction in tumors are triggered by a common mechanism, Science, vol. 363, no. 6435 (2019). See also Basu et al., Epigenetic reprogramming of cell identity: lessons from development for regenerative medicine, Clin. Epigenetics, vol. 13, no. 1, pp. 1-11 (2021).

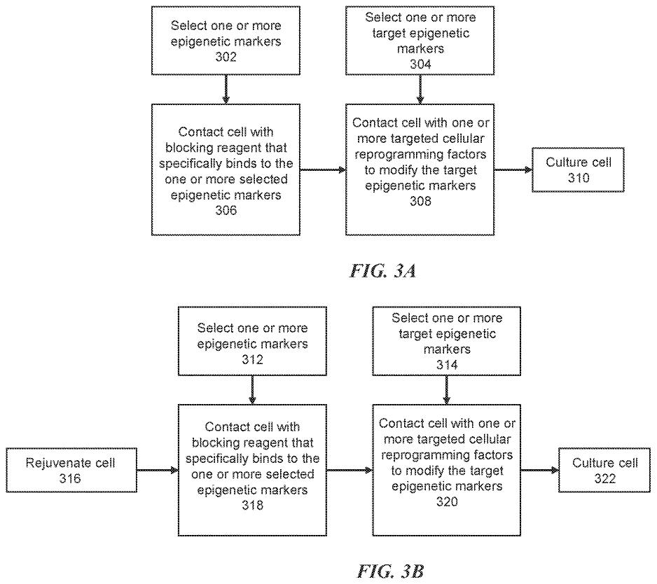

FIG. 3A shows an exemplary method for partially reprogramming a cell. Although the figure is shown representing steps in a particular order, the illustrated steps may be performed in any suitable order. As shown in FIG. 3A, at 302, one or more epigenetic markers are selected. The one or more epigenetic markers may be associated (i.e., correlated), for example, with the identity of the cell subject to the partial reprogramming method. At 304, one or more target epigenetic markers are selected. The one or more target epigenetic markers are those epigenetic markers intended to be modified, for example an epigenetic marker associated with biological aging or a disease state, At 306, a blocking reagent that specifically binds to the one or more selected epigenetic markers is contacted with the cell. In some embodiments, the blocking reagent is added to a cellular medium containing the cell. In some embodiments, the blocking reagent is expressed in the cell, for example using a heterologous vector controlled by an inducible promoter. Exemplary forms of the blocking agent may include mRNA, integrative DNA, non-integrative DNA, and/or proteins. Exemplary methods of introducing the blocking reagent into the cell include (1) passive uptake through the media, (2) transfection, (3) transduction (e.g., using various viruses, lentivirus, AAV, etc.), (4) activation of endogenous genes, and (5) lipid nanoparticles. For example, dCAS9 with guide RNAs may be used for specific markers may be introduced into the cell through transduction using AAV2. In another example, dCAS9 protein and guide RNAs are introduced into the cell directly through electroporation. At 308, the cell is contacted with one or more targeted cellular reprogramming factors to modify the target epigenetic markers. The one or more cellular reprogramming factors may be introduced in the same manner or different manner as the blocking agent. For example, in some embodiments, the one or more cellular reprogramming factors are added to a cellular medium containing the cell. In some embodiments, the one or more cellular reprogramming factors are expressed in the cell, for example using a heterologous vector controlled an inducible promoter. Exemplary methods of introducing the cellular reprogramming factors into the cell include (1) passive uptake through the media, (2) transfection, (3) transduction (e.g., using various viruses, lentivirus, AAV, etc.), (4) activation of endogenous genes, and (5) lipid nanoparticles. Although FIG. 3A shows step 306 occurring prior to step 108, these steps may occur in either order or simultaneously. At optional step 310, the cell is cultured in the presence of the blocking reagent and the one or more modification enzymes, which allows the modification enzymes to modify the targeted epigenetic marker while the blocking regent protects the one or more selected epigenetic markers. In some embodiments, in an alternative to step 310, the method may occur in vivo.

FIG. 3B shows an exemplary method for partially reprogramming a cell, which includes at least partially rejuvenating the cell. Although the figure is shown representing steps in a particular order, the illustrated steps may be performed in any suitable order. As shown in FIG. 3B, at 312, one or more epigenetic markers are selected. The one or more epigenetic markers may be associated (i.e., correlated) with the identity of the cell subject to the partial reprogramming method. At 314, one or more target epigenetic markers are selected. The one or more target epigenetic markers are those epigenetic markers intended to be modified, for example an epigenetic marker associated with biological aging or a disease state. At 316 the cell is at least partially rejuvenated, for example by contacting the cell with one or more non-targeted cellular reprogramming factors (e.g., one or more transcription factors, such as one or more Yamanaka factors). Contacting the cell with the one or more non-targeted cellular reprogramming factors can include, for example, adding the one or more non-targeted cellular reprogramming factors to the cell medium containing the cell. In another example, contacting the cell with the one or more non-targeted cellular reprogramming factors can include expressing the one or more transcription factors in the cell, for example using a heterologous vector controlled an inducible promoter. Exemplary methods of introducing the non-targeted cellular reprogramming factors into the cell include (1) passive uptake through the media, (2) transfection, (3) transduction (e.g., using various viruses, lentivirus, AAV, etc.), (4) activation of endogenous genes, and (5) lipid nanoparticles. At 318, a blocking reagent that specifically binds to the one or more selected epigenetic markers is contacted with the cell. In some embodiments, the blocking reagent is added to a cellular medium containing the cell. In some embodiments, the blocking reagent is expressed in the cell, for example using a heterologous vector controlled by an inducible promoter. Exemplary forms of the blocking agent may include mRNA, integrative DNA, non-integrative DNA and/or proteins. Exemplary methods of introducing the blocking reagent into the cell include (1) passive uptake through the media, (2) transfection, (3) transduction (e.g., using various viruses, lentivirus, AAV, etc.), (4) activation of endogenous genes, and (5) lipid nanoparticles. For example, dCAS9 with guide RNAs may be used for specific markers may be introduced into the cell through transduction using AAV2. In another example, dCAS9 protein and guide RNAs are introduced into the cell directly through electroporation At 320, the cell is contacted with one or more targeted cellular reprogramming factors to modify the target epigenetic markers. In some embodiments, the one or more modification enzymes or fragments are added to a cellular medium containing the cell. In some embodiments, the one or more modification enzymes or fragments are expressed in the cell, for example using a heterologous vector controlled an inducible promoter. Exemplary methods of introducing the targeted cellular reprogramming factors into the cell include (1) passive uptake through the media, (2) transfection, (3) transduction (e.g., using various viruses, lentivirus, AAV, etc.), (4) activation of endogenous genes, and (5) lipid nanoparticles. Although FIG. 3B shows step 316 occurring prior to step 318, and step 318 occurring prior to step 320, these steps may occur in either order or simultaneously. At optional 322, the cell is cultured in the presence of the blocking reagent and the one or more modification enzymes, which allows the modification enzymes to modify the targeted epigenetic marker while the blocking regent protects the one or more selected epigenetic markers. In some embodiments, in an alternative to step 322, the method may occur in vivo.

The cell may be, for example, a fibroblast, a keratinocyte, a peripheral mononuclear blood cell, a hepatocyte, or an epithelial cell. In some embodiments, the cell is a neural cell, a blood cell, an immune cell, a hepatocyte, a lung cell, a pancreatic beta-cell, a cardiomyocyte, or an oligodendrocyte. The cell is obtained from an individual (i.e., is not a cell line). As further described herein, the methods can include protecting one or more epigenetic markers from modification, thus allowing the status for selected epigenetic markers to be maintained.

The one or more epigenetic markers may comprise one or more CpG sites and/or one or more histones. The one or more target epigenetic markers are modified by methylation, demethylation, acetylation, or deacetylation.

The method may include at least partially reversing cellular identity of the cell. In some embodiments, at least partially reversing cellular identity of the cell comprises generating an induced pluripotent step cell (iPSC) from the cell. In some embodiments, at least partially reversing cellular identity of the cell excludes generating an induced pluripotent step cell (iPSC) from the cell. For example, the method may include contacting the cell with one or more cellular reprogramming factors for a limited time (for example, 1-10 days, or 1-20 days) instead of a full reprogramming cycle (generally 20-30 day treatment), or contacting the cell with one or more cellular reprogramming factors at a reduced dose or dose cycling (e.g., on/off cycles). See, for example, Gill et al., Multi-omic rejuvenation of human cells by maturation phase transient reprogramming, Elife, vol. 11, e71624 (2022); Ocampo et al., In Vivo Amelioration of Age-Associated Hallmarks by Partial Reprogramming, Cell, vol 167, pp, 1719-1733.e12 (2016), For example, at least partially reversing cellular differentiation of the cell can include contacting the cell with one or more transcription factors, or inducing or modulating expression of one or more transcription factors, in the cell (e.g., one or more Yamanaka factors, such as OCT4, SOX2, KIF4, and/or c-MYC). Expression of the one or more transcription factors may be modulated or induced by modifying ore or more target epigenetic markers associated with expression of the one or more transcription factors. In another example, expression of the one or more transcription factors using a heterologous expression vector (for example, a PiggyBac gene expression vector a viral expression vector, such as a cytomegalovims (CMV) expression vector). Exemplary methods of introducing the cellular reprogramming factors into the cell include (1) passive uptake through the media, (2) transfection, (3) transduction (e.g., using various viruses, lentivirus, AAV, etc.), (4) activation of endogenous genes, and (5) lipid nanoparticles. In some embodiments, the cell is an immune cell, and at least partially reversing cellular identity of the cell comprises culturing the cell in a high potassium medium (for example, comprising about 40 mM potassium or higher, such as between about 40 mM and about 80 mM potassium). See, for example, Vodnala et al., T cell stemness and dysfunction in tumors are triggered by a common mechanism, Science, vol. 363, no. 6435 (2019); and WO 2021/222479A1.

To modify the one or more target epigenetic markers, the cell is contacted with one or more modification enzymes (or an active fragment thereof). The modification enzymes may be specifically targeted to the one or more target epigenetic markers. Exemplary modification enzymes (also known as “effector proteins”) include KRAB, VPR, p65 VP64, HSF1, p300, DNMT3A, TET1, EZH2, G9a SUV39H1, HDAC3, LSD1, PRDM9, DOT1L, FOG1, BAF, PYL1, ABI1, CIBN, ADAR2, METTL3, METTL14, ALKBH5, and FTO. The modification enzyme may be bound or fused to a nuclease-deficient targeted DNA binding protein.

The targeted epigenetic makers in the cell may be modified using a CRISPR-based editing platform. Exemplary methods for using editing epigenomic markers using a CRISPR-based editing platform are described in Nakamura et al., CRISPR technologies for precise epigenome editing, Nature Cell Biology, vol. 23, pp. 11-22 (2021); Kang et al., Regulation of gene expression by altered promoter methylation using a CRISPR Cas9-mediated epigenetic editing system, Scientific Repots, vol. 9, no. 11960 (2019); Nunez et al., Genome-wide programmable transcriptional memory by CRISPR-based epigenome editing, Cell, vol. 184, p. 2503-2519 (2021); Policarpi et al., Epigenetic editing: Dissecting chromatin function in context, Bioessays, vol. 43, no. 2000316 (2021). In an exemplary method, the CRISPR-based editing platform comprises one or more single guide RNA (sgRNA) molecules that targets an epigenetic marker. A dead Cas9 endonuclease (e.g., Sa/pdCas9) or other suitable ortholog (e.g., dead Cpf1, dead Cas13 or dead CasRx) may be used for the CRISPR-based editing platform, which is optionally introduced using a viral inducible vector. The dead Cas9 endonuclease may be fused to an epigenetic modification protein (which may be an effector protein) or active fragment thereof. Exemplary effector proteins include KRAB, VPR, p65 VP64, HSF1, p300, DNMT3A, TET1, EZH2, G9a, SUV39H1, HDAC3, LSD1, PRDM9, DOT1L, FOG1, BAF, PYL1, ABT1, C1BN, ADAR2, METTL3, METTL14, ALKBH5, and FTO.

In another example, the nuclease-deficient targeted DNA binding protein comprises a transcription activator-like (TAL) effector DNA-binding domain or a zinc finger DNA binding domain that specifically bind the targeted epigenetic marker.

Ideally, epigenetic modification by contacting the cell with one or more cellular reprogramming factors to modify targeted epigenetic markers limits epigenetic modification to only those targeted markers. More commonly, however, the cellular reprogramming factors, particularly non-targeted cellular reprogramming factors, modify non-targeted epigenetic markers. To limit off target epigenetic modification the cell can be contacted with a blocking reagent that specifically binds to one or more selected epigenetic markers. By specifically binding a selected epigenetic marker, the modification enzymes are sterically prevented from modifying the protected marker.

The blocking reagent can include a DNA binding protein that specifically binds to a selected epigenetic marker. The DNA binding protein may specifically bind based on the nucleic acid sequence at the epigenetic locus (that is, the DNA binding protein can bind to the locus irrespective of the status of the epigenetic marker). The DNA binding protein is generally a nuclease-deficient targeted DNA binding protein. For example, DNA binding protein may not include a nuclease domain or, if it includes a nuclease domain said nuclease domain is deficient. The blocking reagent may include a CRSPR-based editing platform, which can include a dead endonuclease domain (e.g., a dead Cas9) domain. The CRISPR-based editing platform of the blocking reagent may further include one or more single guide RNA (sgRNA) molecules that targets one or more epigenetic markers. In another example, the nuclease-deficient targeted DNA binding protein comprises a transcription activator-like (T A L) effector DNA-binding domain or a zinc finger DNA binding domain that specifically bind the selected epigenetic marker.

In contrast to the DNA binding protein used with the one or more modification enzymes (or fragment thereof) used to modify one or more target epigenetic markers, as further described herein, the DNA binding protein used with the blocking reagent is not fused or bound to a modification enzyme.

### Minimizing Modifications to an Off-Target Cell/Tissue

When selecting a target genomic site for epigenetic editing, such as for the purpose of modifying a cellular state, it may be desirable to control the effects of epigenetic editing to specific target cell types and minimize modifications to off-target cell types/tissues. The present disclosure provides long-range epigenetic profiling methods to generate epigenetic maps to identify target genomic sites for epigenetic editing a target cell that can minimize the risk or level of modifications in an off-target cell or tissue. For example, specific genomic sites may be unmethylated in the target cell and methylated in an off-target cell. Targeting this specific genomic site for methylation would produce no change to the genomic site in the off-target cell, since it is already methylated. The methods described herein may be useful to narrow or remove the search space for target epigenetic sites for selective editing.

In some cases, the present disclosure provides methods for generating a target cellular epigenetic map of a target cell, wherein the target cellular epigenetic map provides a methylation state of each genomic site of a plurality of genomic sites in the target cell. In some cases, the method further comprises generating an off-target cellular epigenetic map of an off-target cell, wherein the off-target cellular epigenetic map provides a methylation state of each genomic site of a plurality of genomic sites in the off-target cell. In some cases, the target cell is of a first cell type, and the off-target cell is of a second cell type, wherein the first cell type and the second cell type are different cell types. In some cases, the target cell is from a target tissue and the off-target cell is from an off-target tissue, wherein the target tissue and the off-target tissue are different tissues. For example, in one application, a liver hepatocyte may be selected as a target cell, A pancreatic acinar cell or a gastric epithelial cell may be considered an off-target cell.

In some cases, the method further comprises comparing the target cellular epigenetic map and the off-target cellular epigenetic map, thereby detecting a differential. In some cases, the method farther comprises using the differential to identify a target genomic site in the plurality of genomic sites, wherein (i) the target genomic site is a first methylation state in the target cell, and (ii) the target genomic site is in a second methylation state in the off-target cell, wherein the first methylation state and the second methylation state are different methylation states. For example, a target cellular epigenetic map of a target diseased liver hepatocyte may be compared with an off-target cellular epigenetic map of a healthy pancreatic acinar cell. This comparison may reveal a promoter site that is unmethylated in the target diseased liver hepatocyte and that is methylated in the off-target healthy pancreatic acinar cell. In this example, the promoter site may be identified as a favorable epigenetic editing site for methylation, since a targeted epigenetic modulator comprising a methylase would modify this site in the target diseased liver hepatocyte but would produce no change to this site in the off-target healthy pancreatic acinar cell, since it is already methylated.

In some cases, the method comprises generating a plurality of off-target cellular epigenetic maps of a plurality of off-target cells, wherein the plurality of off-target cellular epigenetic maps provides a methylation state of each genomic site of the plurality of genomic sites in each off-target cell in the plurality of off-target cells. In some cases, the target cell is of a first cell type, and each off-target cell of the plurality of off-target cells is of a cell type that is different from the first cell type. In some cases, the plurality of off-target cells comprises at least two off-target cells of different cell types. In some cases, the target cell is from a target tissue and the plurality of off-target cells is from off-target tissues, wherein the target tissue and the off-target tissues are different tissues. For example, the target cell may be a liver hepatocyte and the plurality of off-target cells may comprise a pancreatic acinar cell or a gastric epithelial cell. In some cases, the plurality of off-target cells comprises a pancreatic acinar cell and a gastric epithelial cell.

In some cases, the method comprises comparing the target cellular epigenetic map and the plurality of off-target cellular epigenetic maps. In some cases, comparing the epigenetic maps detects a differential between the target cellular epigenetic map and the plurality of off-target cellular epigenetic maps. In some cases, the method comprises using the differential to identify the target genomic site in the plurality of genomic sites, wherein the target genomic site is in the second methylation state in each off-target cell in the plurality of off-target cells. For example, the target cellular epigenetic map may be a diseased liver hepatocyte epigenetic map, and the plurality of off-target cellular epigenetic maps may be a healthy pancreatic acinar epigenetic map and a healthy gastric epithelial cell epigenetic map. Comparing the diseased liver hepatocyte epigenetic map with the healthy pancreatic acinar epigenetic map and the healthy gastric epithelial cell epigenetic map may reveal a target site that is unmethylated in the diseased liver hepatocyte and methylated in both the healthy pancreatic acinar and the healthy gastric epithelial cell. This target site may be identified as a favorable target site for methylation given that this target site is already methylated in the healthy pancreatic acinar and the healthy gastric epithelial cell and introducing a targeted methylating agent to this site would have no effect on this site in the healthy pancreatic acinar and the healthy gastric epithelial cell.

### Modified or Edited Cells

In some cases, the method produces a modified cellular state that is functionally more similar to a desired cellular state than the initial cellular state is to the desired cellular state. For example, introducing an epigenetic edit in an initial diseased cell can change the diseased cell to be functionally more similar to a desired healthy state. As another example, introducing an epigenetic edit in an initial highly differentiated cell can change the differentiation state of the cell to a less differentiated state. In some cases, the method further comprises profiling a function of the modified cell, for example, using a functional assay.

In some cases, the method produces a modified cell that exhibits a modified phenotype that is different from an initial phenotype of the target cell. A phenotype of the cell can be expression of a cell marker, a cell size, or cellular morphology. In some cases, the modified phenotype is more similar to a desired phenotype of the desired cell in the desired cellular state than the initial phenotype is to the desired phenotype. For example, if a naïve T-cell is the desired cellular state, introducing an epigenetic edit in an effector T-cell cell can result in the cell exhibiting a desired cell marker characteristic of naïve T-cells. In some cases, the method further comprises profiling a phenotype of the modified cell. For example, expression of a cellular marker can be profiled using antibodies against the cellular marker and flow cytometry analysis. The size or morphology of modified cells can be profiled by imaging.

In some cases, modifying the target genomic site from the initial methylation state to the desired methylation state turns on expression of a gene. In some cases, modifying the target genomic site from the initial methylation state to the desired methylation state turns off expression of a gene. For example, methylating a promoter site can turn off expression of a gene. On the other hand, demethylating a promoter site can turn on expression of a gene. In sone cases, methylating an internal region of a gene can turn on or turn off expression of a gene. In some cases, demethylating an internal region of a gene can turn on or turn off expression of a gene. In some cases, methylating an activator or repressor gene can turn on or turn off expression of a second gene. In some cases, demethylating an activator or repressor gene can turn on or turn off expression of a second gene.

In some cases, the method further comprises epigenetic profiling the modified cell to examine the effects of the epigenetic modulator. Epigenetic profiling of the cell after modification can be used to further refine the epigenetic editing system. For example, for a CRISPR based epigenetic editing system, one or more guide RNAs can be screened for efficacy of epigenetic editing of the target site. The one or more guide RNAs can also be screened for off-target edits at off-target genomic sites.

### Blocking Reagent

In some aspects, the present disclosure provides a blocking reagent. The blocking; reagent can be capable of blocking an off-target genomic site from an epigenetic modification. The blocking reagent can include a nucleic acid binding moiety that is capable of specifically binding to an off-target genomic site, e.g., an epigenetic cellular identity marker. The nucleic acid binding moiety may be configured to bind based on the nucleic acid sequence at the epigenetic locus (that is, the nucleic acid binding moiety can bind to the locus irrespective of the status of the epigenetic marker). The nucleic acid binding moiety can be a nuclease-deficient targeted nucleic acid binding moiety. The blocking reagent may include a CRISPR-based editing platform, which can include a dead endonuclease domain (e.g., a dead Cas9) domain. The CRISPR-based editing platform of the blocking reagent may further include one or more single guide RNA (sgRNA) molecules that targets one or more epigenetic cellular identity markers, e.g., a blocking guide RNA, A blocking guide RNA can comprise a nucleic acid sequence that is complementary to the off-target genomic site identified by any of the methods described herein. In some cases, the blocking guide RNA is configured to bind to a CRISPR/Cas domain, wherein the CRISPR/Cas domain-blocking guide RNA complex hinds to the off-target genomic site. The CRISPR/Cas domain can be catalytically inactive. In some cases, CRISPR/Cas domain-blocking guide RNA complex prevents a modification, e.g., methylation, demethylation, acetylation, or acetylation, from occurring at the off-target genomic site. In another example, the nuclease-deficient targeted nucleic acid binding moiety comprises a transcription activator-like effector (TALE) DNA-binding domain or a zinc finger nucleic acid binding moiety that specifically bind the off-target genomic site, e.g., an epigenetic cellular identity marker. In some cases, the nucleic acid binding moiety used with the blocking reagent is not fused or bound to an modification enzyme.

### Blocking Modifications at Off-Target Genomic Sites

In another aspect, the present disclosure provides a method of blocking a modification at an off-target genomic site. An off-target genomic site can be a genomic site that is unintentionally targeted or a site where a modification is undesired. In some cases, an off-target genomic site comprises an epigenetic cellular identity marker. An epigenetic cellular identity marker can be correlated with the identity (i.e., cellular differentiation state) of cell, as described elsewhere herein. In some cases, loss of the epigenetic cellular identity markers causes the cell to lose its cellular identity. Cell identity can be dictated by the specific set of genes expressed and proteins produced in the cell that are activated by the epigenetic state of the cell to enable its unique function. Altering the epigenetic state of the epigenetic cellular identity markers can cause a loss of cellular state identity. To preserve the identity of the cell, the methods described herein can preserve the epigenetic state of the one or more epigenetic cellular identity markers, e.g., through blocking a modification at an off-target genomic site comprising a cellular identity marker.

To limit off target epigenetic modification, and more particularly to preserve selected epigenetic cellular identity markers, the cell can be contacted with a blocking reagent that specifically binds to one or more selected epigenetic cellular identity markers. By specifically binding a selected epigenetic marker, the modification enzymes are sterically prevented from modifying the protected marker. Thus, the cellular identity of the cell may be preserved when epigenetic cellular identity markers are protected by the blocking reagent.

The blocking reagent can include a nucleic acid binding moiety that specifically binds to an off-target genomic site, e.g., an epigenetic cellular identity marker. The nucleic acid binding moiety may specifically bind based on the nucleic acid sequence at the epigenetic locus (that is, the nucleic acid binding moiety can bind to the locus irrespective of the status of the epigenetic marker). The nucleic acid binding moiety can be a nuclease-deficient targeted nucleic acid binding moiety. The blocking reagent may include a CRISPR-based editing platform, which can include a dead endonuclease domain (e.g., a dead Cas9) domain. The CRISPR-based editing platform of the blocking reagent may further include one or more single guide RNA (sgRNA) molecules that targets one or more epigenetic cellular identity markers, e.g., a blocking guide RNA. A blocking guide RNA can comprise a nucleic acid sequence that is complementary to the off-target genomic site identified by any of the methods described herein. In some cases, the blocking guide RNA is configured to bind to a CRISPR/Cas domain, wherein the CRISPR/Cas domain—blocking guide RNA complex binds to the off-target genomic site. The CRISPR/Cas domain can be catalytically inactive. In another example, the nuclease-deficient targeted DNA binding domain comprises a transcription activator-like effector (TALE) nucleic acid binding moiety or a zinc finger nucleic acid binding moiety that specifically bind the off-target genomic site, e.g., an epigenetic cellular identity marker. In some cases, the CRISPR/Cas domain—blocking guide RNA complex, the TALE nucleic acid binding moiety, or the zinc finger nucleic acid binding moiety prevents a modification, e.g., methylation, demethylation, acetylation, or acetylation, from occurring at the off-target genomic site. In some cases, the nucleic acid binding moiety used with the blocking reagent is not fused or bound to an epigenetic modulator.

### Evaluation of Cellular Reprogramming

The epigenetic profiling method described herein may be used to evaluate a cell undergoing or having undergone cellular reprogramming. In some embodiments, the epigenetic profiling method described herein is used for evaluating a cellular reprogramming protocol. Cells in a cell population may be subject to epigenetic reprogramming with the intention of obtaining a target epigenetic profile.

A cellular reprogramming protocol may be selected to reprogram a cell to best match a target cell (which may be a real cell or a hypothetical cell). The target cell has a target epigenetic profile, which can include an epigenetic status of one or more epigenetic cellular identity markers. The target epigenetic profile may also include an epigenetic status of one or more target epigenetic markers. The target epigenetic profile need not include the statuses of all epigenetic markers of the target cell; for example, certain epigenetic markers may not significantly alter the cell's identity or age/disease status. The selected protocol optimally modifies the epigenetic markers of the cell being modified to best match the target cell.

The target epigenetic profile is the epigenetic profile of a cell (either real or theoretical) desired to be matched according to the optimized cellular reprogramming protocol. The target epigenetic profile may be selected, for example, from a database of epigenetic profiles or empirically determined. For example, the epigenetic profile of a target cell may be determined (for example, using a methylation sequencing (methyl-seq) method). Exemplary profiling techniques may include, for example, epigenetic profiling, transcriptomic profiling, proteomic profiling, cell imaging, determining a cellular state, a functional assay, multi-omics profiling, metabolic profiling, flow cytometry, whole genome bisulfite sequencing, single-cell sequencing, ATAC sequencing, single-cell ATAC sequencing, a methylation microarray profiling, methylation sequencing, single-cell methylation sequencing, single-cell RNA sequencing, or nucleic acid sequencing. In some implementation, the target cell is profiled using single-cell sequencing, methylation sequencing, or single-cell methylation sequencing.

The target cell can include a desired identify characteristic (e.g., a particular type of cell) and can include one or more additional desired phenotypes (for example, a desired age or desired disease status associated with an epigenetic profile). The target epigenetic profile can include one or more cellular identity markers and an associate maker status for each of the one or more cellular identity markers. The target epigenetic profile may further include one or more target epigenetic markers and an epigenetic status of the one or more target epigenetic markers. The target epigenetic markers are markers other than the one or more cellular identity markers that are associated with the desired phenotype of the cell. For example, the one or more target epigenetic markers may be associated (i.e., correlated) with a biological age or disease state.

A differential between the target epigenetic profile and an epigenetic profile from a cell in a cell population (for example obtained according to the methods described herein) can be obtained, thereby providing a differential epigenetic profile. The differential epigenetic profile indicates differences between the target epigenetic profile and the test epigenetic profile. Thus, it is possible to obtain, for example, a differential epigenetic profile at each of the plurality of time points, and for each cell sample in the plurality of cell samples. By analyzing the differential epigenetic profile, it is possible to determine how close a particular reprogramming protocol is to obtaining the target epigenetic profile at a particular time point.

### Epigenetic Modulators

As described herein, the present disclosure in part provides an epigenetic modulator. In some embodiments, the modulator increases or decreases the expression of a target gene, e.g., a transcription factor. In some embodiments, the modulator suppresses the expression and/or activity of a target gene. In some embodiments, the modulator increases the expression and/or activity of a target gene.

In some embodiments, the epigenetic modulator comprises a nuclear binding domain. In some embodiments, the nucleic acid binding domain can be a CRISPR/Cas domain, a zinc finger domain, or a TAL domain. In some embodiments, the nucleic acid binding domain is fused to an effector moiety (e.g., DNA methyltransferase, DNA demethylase, a histone methyltransferase, a histone demethylase, a histone acetyltransferase, or a histone deacetylase).

In some embodiments, the effector moiety of the epigenetic modulator may be or may comprise a moiety capable of modifying a nucleic acid. In some embodiments, the nucleic acid is a DNA, e.g., genomic DNA, In some embodiments, the nucleic acid is a RNA, e.g., mRNA.

In some embodiments, the effector moiety is capable of altering methylation profile of a genome of a cell. In some cases, effector moiety can modify a nucleic acid by increasing or decreasing methylation in a target nucleic acid. In other cases, the effector moiety modifies the chromatin structure of a cell through histone modifications, e.g., via modulating histone methylation and/or acetylation profile. In some embodiments, the epigenetic modulator comprises a nucleic acid binding moiety and multiple effector moieties (e.g., 1, 2, 3, 4, 5, 6, 7, 8, 9, or 10 effector moieties). In some embodiments, the nucleic acid binding moiety and the effector moiety are covalently linked, e.g., via a peptide bond. In some embodiments, the nucleic acid binding moiety and the effector moiety are not covalently linked.

In some embodiments, the epigenetic modulator may be capable of binding to a transcription regulatory element (e.g., a promoter, an enhancer, or a transcription start site operably linked to a gene) and facilitating an epigenetic modification at the desired target site. In some embodiments, the epigenetic modulator may be capable of binding to a site in a CpG island of a target nucleic acid and introducing an epigenetic modification at a desired target site. In some embodiments, the epigenetic modulator may be capable of methylating or demethylating at least one CpG site of a target nucleic acid.

In some embodiments, the epigenetic modulator is capable of binding to a transcription regulatory element. In some embodiments, the epigenetic modulator is capable of binding to a transcription regulatory element selected from a promoter, an enhancer, a silencer, an insulator, a locus control region, or a transcription start site operably linked to a gene. In some embodiments, the epigenetic modulator is capable of binding to a promoter element. In some embodiments, the epigenetic modulator is capable of binding to a promoter element selected from a TATA box, a CAAT box, a GC box, an INR, a DPE, an MTE, a DCE, or a BRE. In some embodiments, the epigenetic modulator is capable of binding to a TATA box. In some embodiments, the epigenetic modulator is capable of binding to a CA AT box. In some embodiments, the epigenetic modulator is capable of binding to a GC box. In some embodiments, the epigenetic modulator is capable of binding to an INR. In some embodiments, the epigenetic modulator is capable of binding to a DPE. In some embodiments, the epigenetic modulator is capable of binding to an MTE. In some embodiments, the epigenetic modulator is capable of binding to a DCE. In some embodiments, the epigenetic modulator is capable of binding to a BRIE. The consensus sequences of exemplary promoter elements are provided in Table 1 below. In some embodiments, the promoter may be constitutively active. Alternatively, in some embodiments, the promoter may be conditionally active (e.g., where transcription is initiated only under certain physiological conditions). In some embodiments, the epigenetic modulator is capable of binding to an enhancer. In some embodiments, the epigenetic modulator is capable of binding to a silencer. In some embodiments, the epigenetic modulator is capable of binding to an insulator. In some embodiments, the epigenetic modulator is capable of binding to a locus control region. In some embodiments, the epigenetic modulator is capable of binding to a transcription start site.

TABLE 1
Exemplary Promoter Elements

| Promoter Element | Consensus Sequence |
|---|---|
| TATA box | TATAWAWN |
| CAAT box | GGCCAATCT |
| GC box | GGGCGG |
| INR | YYANWYY |
| DPE | RGWYVT |
| MTE | CSARCSSAAC |
| DCE sub element 1 | CTTC |
| DCE sub element 2 | CTGT |
| DCE sub element 3 | AGC |
| BRE | SSRCGCC |

In some embodiments, a nucleic acid binding moiety binds to its target sequence with a KD of less than or equal to 500, 450, 400, 350, 300, 250, 200, 150, 100, 50, 40, 30, 20, 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.005, 0.003, 0.002, or 0.001M. In some embodiments, a nucleic acid binding moiety does not bind, e.g., does not detectably bind to a non-target sequence. In some embodiments, the nucleic acid binding moiety comprises a sequence complimentary, e.g., at least 5%, at least 10%, at least 15%, at least 20%, at least 30%, at least 40%, at least 50%, at least 60%, at least 70%, at least 80%, at least 90%, at least 99%, or 100% complimentary to the target sequence.

In some cases, an epigenetic modulator may comprise a fusion protein comprising a nucleic acid binding domain and an effector domain. In some instances, the nucleic acid binding domain of an epigenetic modulator may be located at the N-terminus or C-terminus of the effector domain. In some cases, the nucleic acid binding domain is located at the N-terminus of the effector domain. In other cases, the nucleic acid binding domain is located at the C-terminus of the effector domain. In some cases, the nucleic acid binding domain is located within the effector domain. In other cases, the effector domain is located within the nucleic acid binding domain. In some embodiments, the epigenetic modulator comprises more than one effector domain. For an epigenetic modulator comprising more than one effector domain, in some cases, the first effector domain may be located at the N-terminus or C-terminus of the second effector domain. In other cases, first effector domain may be located at the N-terminus of the nucleic acid binding domains, and the second effector domain may be located at the C-terminus of the nucleic acid binding domain. The epigenetic modulator may comprise any combination of arrangements of the nucleic acid binding moiety and the effector moiety described in this disclosure.

In some embodiments, the epigenetic modulator. e.g., an epigenetic modulator described herein may be capable of methylation, demethylation, acetylation, and/or deacetylation. In some embodiments, the epigenetic modulator is capable of adding or removing a methyl group in a nucleic acid. In some embodiments, the epigenetic modulator is capable of adding or removing a methyl group in a histone. In sone embodiments, the epigenetic modulator is capable of adding or removing an acetyl group in a histone. In some embodiments, the epigenetic modulator is an epigenetic modulator comprising an effector moiety selected from DNMT3A1, DNMT3A2. DNMT3B1, DNMT3B2, DNMT3B3, DNMT3B4, DNMT3B5, DNMT3B6, DNMT3L, TRDMT1, MQ1, MET1, DRM2, CMT2, CMT3, TET1, TET2, TET3, SETDB1, SETDB2, EHMT2 (i.e., G9A), EHMT1 (i.e. GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, NO66, KAT1, KAT2A, KAT3A, KAT3B, KAT13C, HDAC1, HDAC2, HDAC3, HDAC4, HDAC5, HDAC6, HDAC7 HDAC8, HDAC9, HDAC10, HDAC11, SIRT1, SIRT2, SIRT3, STRT4, SIRT5, SIRT6, SIRT7, SIRT8, SIRT9, KRAB, MeCP2, HP1, RBBP4, REST, FOG1, SUZ12, MBD2, MBD3, TDG, ROS1, DME, DML2, DML3, TRDMT1 (DNMT2), m.MpeI, M.SssI, M. HpaII, M.AluI, M.HacIII, M.HhaI, M.Msp1, MET1, Dim-2, dDnmt2, or Pmt1 or a functional equivalent thereof. In some cases, the effector moiety comprises M.TaqI, M.EcoDam, M.CerMI, or CamA.

In some embodiments, an epigenetic modulator comprises an effector moiety comprising DNMT3A. In some embodiments, an epigenetic modulator comprises an effector moiety comprising DNMT3A and KRAB.

In some embodiments, the epigenetic modulator, e.g., an epigenetic modulator described herein may comprise multiple effector moieties, e.g., at least 2, at least 3, at least 4, at least 5, at least 6, at least 7, at least 8, at least 9, or at least 10 effector moieties.

In some embodiments, the 2nd, 3rd, 4th, 5th, 6th, 7th, 8th, 9th, or 10th effector moiety is selected from one or more of DNMT3A1, DNMT3A2, DNMT3B1, DNMT3B2, DNMT3B3, DNMT3B4, DNMT3B5, DNMT3B6, DNMT3L, TRDMT1, MQ1, MET1, DRM2, CMT2, CMT3, TET1, TET2, TET3, SETDB1, SETDB2, EHMT2 (i.e., G9A), EHMT1 (i.e., GLP), SUV39H1, EZH2, EZH1, SUV39H2, SETD8, SUV420H1, SUV420H2, KDM1A (i.e., LSD1), KDM1B (i.e., LSD2), KDM2A, KDM2B, KDM5A, KDM5B, KDM5C, KDM5D, KDM4B, NO66, KAT1, KAT2A, KAT3A, KAT3B, KAT13C, HDAC1, HDAC2, HDAC3, HDAC4, HDAC5, HDAC6, HDAC7, HDAC8, HDAC10, HDAC10, HDAC11, SIRT1, SIRT2, SIRT3, SIRT4, SIRT5, SIRT6, SIRT7, SIRT8, SIRT9, KRAB, MeCP2, HP1, RBBP4, REST, FOG1, SUZ12, M.TaqI, M.EcoDam, M.CcrMI, CamnA, MBD2, IBD3, TDG, ROS1, DME, DML2, DML3, TRDMT1 (DNMT2), m.MpeI, M.SssI, M. HpaII, M.AluI, M.HaeIII, M.HhaI, M.Msp1, MET1, Dim-2, dDnt2, or Pmt1 or a functional equivalent thereof.

In some embodiments, the epigenetic modulator, e.g., an epigenetic modulator described herein may simultaneously methylate and transcriptionally repress a target site. In some embodiments, the epigenetic modulator, e.g., an epigenetic modulator described herein may simultaneously methylate and transcriptionally activate a target site. In some embodiments, the epigenetic modulator, e.g., an epigenetic modulator described herein may simultaneously demethylate and transcriptionally repress a target site. In some embodiments, the epigenetic modulator, e.g., an epigenetic modulator described herein may simultaneously demethylate and transcriptionally activate a target site. In some embodiments, the epigenetic modulator, e.g., an epigenetic modulator described herein may simultaneously acetylate and transcriptionally repress a target site. In some embodiments, the epigenetic modulator, e.g., an epigenetic modulator described herein may simultaneously deacetylate and transcriptionally activate a target site.

In some embodiments, the effector moiety of the epigenetic modulator may enhance or repress methylation in a target nucleic acid. The effector moiety of the epigenetic modulator may be or comprise a DNA methyltransferase or a functional equivalent thereof. The DNA methyltransferase may be selected from a m6A methyltransferase, an m4C methyltransferase, and an m5C methyltransferase. The DNA methyltransferase may be selected from DNMT1, DNMT3A1, DNMT3A2, DNMT3B1, DNMT3B2, DNMT3B3, DNMT3B4, DNMT3B5, DNMT3B6, DNMT3L, TRDMT1, MQ1, MET1, DRM2, CMT2, CMT3, or a functional equivalent thereof.

In some embodiments, the effector moiety may be or may comprise a moiety capable of effecting DNA demethylation. The effector moiety may be or comprise a DNA demethylase. The effector moiety may comprise a member of the TET family. The effector moiety may be selected from TET1, TET2, and TET3, or a functional equivalent thereof. The effector moiety may be or comprise TDG.

In other embodiments, the effector moiety of the epigenetic modulator may increase or decrease methylation or acetylation in a histone. Increasing or decreasing methylation or acetylation in a histone can modify chromatin structure. In some embodiments, the effector moiety may be or comprise a histone methyltransferase or a functional equivalent thereof. The histone methyltransferase may be selected from SET1, SETDB1, SETDB2, EHMT2 (i.e., G9A), EHMT1 (i.e., GLP) SUV39H1, EZH2, EZH1, SUV39H2, SETD8, SUV420H1, SUV420H2, a viral lysine methyltransferase (vSET), a histone methyltransferase (SET2), a protein-lysine N-methyltransferase (SMYD2), or a functional equivalent thereof. In some cases, the effector moiety comprises DOT1L, PRDM9, PRMT1, PRMT2, PRMT3, PRMT4, PRMT5, NSD1, NSD2, NSD3, ROM2, AtHD3A, HDAC11, HDAC8, SIRT3, SIRT6, HST2, a SETDB1 domain, a NuRD domain, or a TET family protein domain.

The effector moiety of the epigenetic modulator may be or comprise a histone demethylase or a functional equivalent thereof. The histone demethylase may be selected from KD1A (i.e., LSD1), KDM1B (i.e., LSD2), KDM2A, KDM2B, KDM5A, KDM5B, KDM5C, KDM5D, KDM4B, NO66, UTX, JMJD3, or a functional equivalent thereof.

In some embodiments, the effector moiety of the epigenetic modulator may, be capable of adding or removing an acetyl group in a histone. In some embodiments, the effector moiety of the epigenetic modulator may be or comprise a histone acetyltransferase or a functional equivalent thereof. The histone acetyltransferase may be selected from KAT1, KAT2A, KAT3A, KAT3B, KAT13C, or a functional equivalent thereof. The effector moiety of the epigenetic modulator may be or comprise a histone deacetylase. The histone deacetylase may be selected from HDAC1, HDAC2, HDAC3, HDAC4, HDAC5, HDAC6, HDAC7, HDAC8, HDAC9, HDAC10, HDAC11, SIRT1, SIRT2, SIRT3, STRT4, SIRT5, SIRT6, SIRT7, SIRT8, SIRT9, or a functional equivalent of any thereof.

In some embodiments, the effector moiety of the epigenetic modulator may be or comprise a transcriptional activator moiety or a transcriptional regulator. In some embodiments, the transcriptional activator moiety may be selected from categories comprising a DNA demethylase, historic acetyltransferase, histone methyltransferase, and histone demethylase. In some embodiments, the transcriptional activator moiety or transcriptional regulator may be selected from a VP16 tetramer (e.g., VP64), a p65 activation domain, a VP 160, Rta, a p300 domain, VPR, VPH, HSF1, CBP, FOXO3, a KRAB domain, a lysine-specific histone demethylase 1 (LSD1), a euchromatic histone-lysine N-methyitransferase 2 (G9a), a histone-lysine N-methyltransferase, an enhancer of zeste homolog 2 (EZH2), a viral lysine methyltransferase (vSET), a histone methyltransferase (SET2), a protein-lysine N-methyltransferase (SMYD2), SUV39H1, NUE, DIM5, MES0L04, SET8, SET-TAF1B, an Epstein-Barr virus R transactivator (Rta) activation domain, an Rta activation domain, CACO1, DRM1, DRM2, CMT1, CMT2, CMT3, CBX8, CBX5, CBX1, CBX3, CBX4, CBX7, CDYL2, CDY2, PCGF2, SCMH1, SCML2, MPP8, SUMO3, SUMO1, SUMO5, HERC2, IRF2BP1, IRF2BP2, IRF2BPL, KMT2A, HAT1, HIF1lalpha, SMARCA2, SIN3A, RYBP, SAV1, HAP2, HAP3, or HAP4. In some cases, the effector moiety comprises VPH, VPR, miniVR, or microVR, In some cases, the effector moiety comprises a gene expression regulatory domain. In some cases, the effector moiety comprises Masc1, Masc2, Rid, a domain encoded by the hsdM gene, or a domain encoded by the hsDSgene. In some embodiments, the effector moiety of the epigenetic modulator may be or comprise a transcriptional regulation domain. The transcriptional regulation domain may be selected from Kruppel associated box, such as a KRAB domain, an ERF repressor domain, an MXII repressor domain, a SID repressor domain, a SID4X repressor domain, or a Mad-SID repressor domain. In some cases, the KRAB domain is a KRAB domain of KOX1 or ZIM3.

In sone embodiments, the effector moiety of the epigenetic modulator comprises a transcriptional repressor moiety, e.g., an effector moiety selected from KRAB, MeCP2, HP1, RBBP4, REST1, FOG1, SUZ12, or a functional equivalent.

In some embodiments, the effector moiety of the epigenetic modulator may be or comprise a transcription factor regulator or DNA-binding domain. The transcription factor regulator or DNA-binding domain may be selected from a KRAB domain, KAP1 domain, MECP2 domain, SAM, CTCF, SOX2, KLF4, OCT3/4, XISTA/B/C/D/E/F, VP16, P64, p65, FOXA1, FOXA2, FOXO3, FOXO1, TOX, TOX3, TOX4, ID2, ID1, CREM, SCX, TWST1, CREB1, TERF1, ID3, GSX1, ATF1, TWST2, ZMYM3, I2BP1, RHXF1, I2BL, T168, HXB13, HEY1, PHC2, FIGLA, SAM11, KMT2B, HEY2, JDP2, ASCL4, HHEX, GSX2, ASCL3, PHC1, OTP, I2BP2, VGLL2, HXA11, PDLI4, ASCL2, CDX4, ZN860, NKX25, ISL1, CDX2, PROP1, HXC11, HXC10, PRKS6A, VSX1, NKX23, MTG16, HMX3, HMX1, KIF22, CSTF2, CEBPE, CLX2, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CDX8, CEBPD, DLX5, NOTC1, TERF2, RGS12, PAX7, NKX62, ASXL2, GATA1, ZMYM5, GATA2, GATA3, IRX4, ZBED6, LHX4, NKX61, R51A1, MB3L1, NKX22, ATF1, SSX2, ZN680, HXA13, PHC3, TCF24, ETV7, LMBL4, PDIP3, CERBPB, SIN3B, SMBT1, SEC13, FIP1, ALX4, LHX3, PRIC2, MAGI3, NELL1, PRRX1, MTG8R, RX2, DLX3, DLX1, NKX26, NAB1, SAMD7, PITX3, WDR5, MEOX2, NAB2, DRX8, FOXA2, EMX2, CPSF6, HXC12, KDM2B, LMBL3, PHX2A, EMX1 NC2B, DLX4, SRY, NELL1, BSH, SF3B4, TEAD1. TEAD2, RGAP1, PHF1, RBBP7, SPI2B, LRP1, MIXL1, SGT1, LMCD1, CEBPA, SOX14, ZTIP, PRP19, NKX11, RBBP4, DMRT2, SMCA2, VP16, VP64, VP160, CITED2, Stat3, p65, p53, ZNF473, myb, CRTC1, Med9, EGR3, Dpy-30, NCOA3, HSF1, YAF2, MGA, BIN1, PTA, AF9, ANM2, APBB1, EGR3, IKKA, ITCH, KIBRA, KPCI, KS6B2, MYB, MYBA, NCOA2, NOCA3, NOC2, STAT2, T2EB, CRTC2, CRTC3, CXXC1, DPF1, ENL, IMA5, MTA3, WWTR1, CREBBP, GCN4, GCN5, SAGA, or SALSA.

In some embodiments, the effector moiety of the epigenetic modulator may comprise a tyrosine kinase, e.g., ABL1 or TK. In some cases, the effector moiety of the epigenetic modulator may comprise a Homobox, e.g., HOXA13, HOXB13, HOXC13, HOXA11, HOXC11, HOXC10, HOXA10, HOXB9, HOXA9.

In some embodiments, the effector moiety of the epigenetic modulator may be or comprise an epigenetic or chromatin modifier. The epigenetic or chromatin modifier may be selected from a TET protein (e.g., TET1), an EKE protein (e.g., ERF1, ERF3), LSD1, PYGO1, KRAB, MeCP2, SIN3A, HDT1, MBD2B, NIPP1, VP64, HP1A, Rb, SUVR4, COBB, NCOR, or HP1A.

In some embodiments, the effector moiety of the epigenetic modulator may be or comprise a protein complex or interactor. The protein complex or interactor may be selected from APC16, DPY30, PRP19, PYGO1, PYGO2, SMCA2, SMRC2, U2AF4, WBP4, WWP1, WWP2, PCAF, RBAK, or HIKR1.

In some embodiments, the effector moiety of the epigenetic modulator may be or comprise a protein domain (e.g., a P16 domain) or a protein tag (e.g., a SunTag).

In some embodiments, the effector moiety may be a durable effector moiety. In some embodiments, the effector moiety may be a transient effector moiety. In some embodiments, the epigenetic modulator may comprise at least two durable effector moieties. In some embodiments, the epigenetic modulator may comprise at least two transient effector moieties. In some embodiments, the epigenetic modulator may comprise at least one durable effector moiety and at least one transient effector moiety.

In some embodiments, an epigenetic modulator comprises a protein having a sequence as recited in Uniprot ref: Q8NFU7 or a protein encoded by a nucleotide sequence as recited in NCBI Accession: Accession: NM_030625.3,
GI: 1519311914; or Accession: NM_001406365.1, GI: 2238345226;
or Accession: NM_001406367.1, GI: 2238345083; or Accession: NM_001406368.1,
GI: 2238345245; or Accession: NM_001406369.1, G&: 2238345201;
or Accession: NM_001406370.1, GI: 2238345031;
or Accession: NM_001406371.1, GI: 2238345008; or Accession: NM_001406372.1,
GI: 2238345087; or Accession: NM_001406373.1, GT: 2238345233;
or Accession: NM_001406374.1, GI: 2238885731; or Accession: NM_001406375.1,
GI: 2238345043: or Accession: NM_001406376.1, GI: 2238345085. In some embodiments, an epigenetic modulator comprises a functional fragment or variant of any thereof, or a polypeptide with a sequence that has at least 80, 85, 90, 91, 92, 93, 94, 95, 96, 97, 98, or 99% identity to any of the above-referenced sequences.

In some embodiments, an epigenetic modulator comprises a protein having a sequence as recited in Uniprot ref: Q9Y6K1 or a protein encoded by a nucleotide sequence as recited in NCBI Accession: NM_001320892.1 GI: 1677500358; or Accession: NM_001320893.1 GI: 1003701584; or Accession: NM_001375819.1 GI: 1034612234; or Accession: NM_0225525, GT: 1812533218; or Accession: NM_153759.3, CT: 371940994; or Accession: NM_175629.2, GI: 371940990; or Accession: NM_175630.1, GI: 28559070. In sone embodiments, an epigenetic modulator comprises a functional fragment or variant of any thereof, or a polypeptide with a sequence that has at least 80, 85, 90, 91, 92, 93, 94, 95, 96, 97, 98, or 99% identity to any of the above-referenced sequences. In some embodiments, the epigenetic modulator can be part of a construct that comprises a Cas9 protein. In some embodiments, the epigenetic modulator methylates the target sequence. In some embodiments, the epigenetic modulator deactivates the target gene.

In some embodiments, an epigenetic modulator comprises a protein having a sequence as recited in Uniprot ref: Q9UJW3 or a protein encoded by a nucleotide sequence as recited in NCBI Accession: NM_013369.4, GI: 1676318741; or Accession: NM_175867.3, GI: 1732746326. In some embodiments, an epigenetic modulator comprises a functional fragment or variant of any thereof, or a polypeptide with a sequence that has at least 80, 85, 90, 91, 92, 93, 94, 95, 96, 97, 98, or 99% identity to any of the above-referenced sequences. In some embodiments, the epigenetic modulator can be part of a construct that comprises a Cas9 protein. In some embodiments, the epigenetic modulator methylates the target sequence. In some embodiments, the epigenetic modulator deactivates the target gene. In some embodiments, an epigenetic modulator comprises a protein having a sequence as recited in Uniprot ref: P21506 or a protein encoded by a nucleotide sequence as recited in NCBI Accession: NM_015394.5, GI: 1519244023. In some embodiments, an epigenetic modulator comprises a functional fragment or variant of an, thereof, or a polypeptide with a sequence that has at least 80, 85, 90, 91, 92, 93, 94, 95, 96, 97, 98, or 99% identity to any of the above-referenced sequences. In some embodiments, the epigenetic modulator can be part of a construct that comprises a Cas9 protein. In some embodiments, the epigenetic modulator methylates the target sequence. In some embodiments, the fusion construct deactivates the target gene.

In some embodiments, the epigenetic modulator further comprises a linker, e.g., a linker connecting the domains of the epigenetic modulator. In some cases, a linker may connect a polypeptide to another polypeptide. In some cases, a linker may connect a polypeptide to a nucleic acid. In some cases, a linker may connect a nucleic acid to another nucleic acid. In some cases, a linker connects the nucleic acid binding domain and the effector domain of an epigenetic modulator. A linker may be a chemical bond. In some cases, a linker may be a covalent bond. In other cases, a linker may be a noncovalent bond. In some cases, a linker may be a peptide linker. In some cases, a peptide linker may be at least 2, 3, 4, 5, 6, 7, 8, 9, 10 or more amino acids in length. In some cases, a linker may be a rigid linker. As well known by one of skill in the art, rigid linkers may comprise an alpha helix structure or Pro-rich sequence. Rigid linkers maintain a substantially fixed spatial distance between domains. In other cases, a linker may be a flexible linker. As well known by one of skill in the art, flexible linkers may comprise small amino acids (e.g., Gly, Ser, or Ala). Flexible linkers allow the domains they connect to have flexibility of movement relative to each other. In some cases, a linker may be a cleavable linker. Cleavable linkers may utilize the reversible nature of a disulfide bond. In some cases, a cleavable linker comprises a cleavage site motif for a protease. In some cases, a cleavable linker may be a self-cleaving linker. In vivo cleavage of linkers in compositions described herein may be cleaved in specific conditions.

In some instances, an epigenetic modulator described herein may comprise one or more nuclear localization sequences (NLS) (e.g., an SV40 NLS). In some cases, the one or more NLS facilitates the import of the epigenetic modulator comprising an NLS into the cell nucleus. In some cases, the epigenetic modulator may comprise 1 NLS. In some cases, the epigenetic modulator may comprise 2 NLSs. In some cases, the polypeptide may comprise 3 NLSs. In other cases, the epigenetic modulator may comprise more than 3, 4, 5, 6, 7, 8, 9, or 10 NLSs. In some cases, the NLS is located at the N-terminus, C-terminus, or in an internal region of the epigenetic modulator. In some cases, an NLS is fused to the N-terminus of the nucleic acid binding domain of an epigenetic modulator described herein. In some cases, an NLS is fused to the C-terminus of the nucleic acid binding domain of an epigenetic modulator. In some cases, an NLS is fused to the N-terminus of the effector domain of an epigenetic modulator. In some cases, an NLS is fused to the C-terminus of the effector domain of an epigenetic modulator. In some cases, the nucleic acid binding domain of the epigenetic modulator does not comprise an NLS In some cases, the effector domain of the epigenetic modulator does not comprise an NLS. In some cases, an NLS is fused to the N-terminus of a CRISPR/Cas effector protein. In some cases, an NLS is fused to the C-terminus of a CRISPR/Cas effector protein. Examples of NLS are provided in Table 2 below.

TABLE 2
Exemplary NLS Sequences

| | | SEQ |
|---|---|---|
| | | ID |
| NLS | Sequence | NO. |
| SV40 Large T-AG | PKKKRKV | 16 |
| EGL-13 | MSRRRKANPTKLSENAKKLAKEVEN | 17 |
| Tus-protein | KLKIKRPVK | 18 |
| polyoma large T-AG | VSRKRPRP | 19 |
| Hepatitis D virus | EGAPPAKRAR | 20 |
| antigen | | |
| Rev protein | RQARRNRRRRWRERNR | 21 |
| murine p53 | PPQPKKKPLDGE | 22 |
| nuceloplasmin | AVKRPAATKKAGQAKKKKLD | 23 |

It should be noted that the epigenetic modulators and effector moieties of the disclosure may be delivered to cells directly as polypeptides, or indirectly via polynucleotide moieties (e.g. DNA, RNA) that may be transcribed and/or translated into polypeptides in the cell.

### CRISPR/Cas Domains

In some embodiments, the nucleic acid binding moiety of the epigenetic modulator determines the site of nucleic acid modification through specific binding with a target nucleic acid. In some embodiments, the nucleic acid binding moiety may be or comprise a CRISPR/Cas domain, a zinc finger domain, or a TAL domain. In some embodiments, the nucleic acid binding moiety of the epigenetic modulator may be or may comprise a Cas9 protein or a functional equivalent. In some embodiments, the nucleic acid binding moiety of the epigenetic modulator may be or may comprise a Cas12 protein or a functional equivalent.

In some embodiments, the CRISPR/Cas domain comprises one or more RNA molecules, which can be a crRNA and/or a tracrRNA and/or optionally, an engineered single guide RNA or sgRNA. In some embodiments, the CRISPR/Cas domain forms a complex with its partner RNA or RNAs. In some embodiments, the CRISPR/Cas domain and RNA complex utilizes RNA-DNA base pairing to determine the binding site to a target nucleic acid. In some embodiments, the CRISPR/Cas domain optionally complexed with its partner sgRNA or sgRNAs binds to a CpG site in a target nucleic acid. In some embodiments, the CRISPR/Cas domain optionally complexed with its partner sgRNA or sgRNAs binds to a protospacer adjacent motif (PAM) sequence in the target nucleic acid. In some embodiments, the PAM sequence is located within a CpG Island in a target nucleic acid.

In some embodiments, the CRISPR/Cas domain may comprise a CRISPR/Cas protein. In some embodiments, a CRISPR/Cas domain may be derived from a protein involved in a Clustered Regularly Interspaced Short Palindromic Repeats (CRISPR) system or have structural and/or functional similarities to a protein involved in the CRISPR system and optionally a guide RNA, e.g., a single guide RNA (sgRNA). Two classes of CRISPR systems have been identified, class 1 and class 2 CRISPR systems. The class 2 CRISPR systems use a single Cas endonuclease effector (rather than a multiple subunit effector). Class 2 CRISPR systems can comprise type II or type V systems. An example of a type 11 CRISPR system uses an effector comprising a Cas9 endonuclease, a CRISPR RNA (“crRNA”), and a trans-activating crRNA (“tracrRNA”). The crRNA contains a “guide RNA”, typically about 20-nucleotide RNA sequence that corresponds to a target DNA sequence. crRNA also contains a region that binds to the tracrRNA to form a double-stranded structure which is cleaved by RNase III, resulting in a crRNA/tracrRNA hybrid. A crRNA/tracrRNA hybrid then directs Cas9 endonuclease to recognize and cleave a target DNA sequence. One example of a type V system comprises the endonuclease Cpf1, which is smaller than Cas9; examples include AsCpf1 (from Acidaminococcus sp.) and LbCpf1 (from Lachnospiraceae sp.). Cpf1-associated CRISPR arrays are processed into mature crRN s without the requirement of a tracrRNA; in other words, a Cpf1 system requires only Cpf1 nuclease and a crRNA to cleave a target DNA sequence. The CRISPR/Cas protein may be selected from a type I, type II, type III, type IV, type V Cas protein, and type VI Cas protein. The CRISPR/Cas protein may be selected from Cas1, Cas1B, Cas2, Cas3, Cas4, Cas5, Cas5d, Cas5t, Cas5h, Cas5a, Cas6, Cas7 Cas8, Cas8a, Cas8b, Cas8c, Cas9, Cas10, Cas10d, Cas12a/Cpf1, Cas12b/C2c1, Cas12c/C2c3, Cas12d/CasY, Cas12e/CasX, Cas12g, Cas12h, Cas12i, Cas12j (Cas-phi2), Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csn6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, AsCas12a, Cas13a, Cas13b, Cas13c, Cas13d, Cas13X, Cas13Y, LbCas12a, HypaCas9, a Type I Cas effector protein, a Type 11 Cas effector protein, a Type III Cas effector protein, a Type IV Cas effector protein, a Type V Cas effector protein, a Type VI Cas effector protein, CARE, DinG, Cpf1, Cas12b/C2c1, Cas12c/C2c3, Cas12b/C2c1, and functional fragments and derivatives thereof. In some embodiments, the CRISPR/Cas protein may be or comprise a Cas9 ortholog. The Cas9 protein may be selected from SpCas9, SaCas9, ScCas9, StCas9, NmCas9, VRERCas9, VERCas9, xCas9, espCas91.0, espCas1.1, Cas9HF1, hypaCas9, evoCas9, HiFiCas9, and CjCas9. In some embodiments, the CRISPR/Cas protein may be or comprise a Cas12 ortholog. The Cas12 protein may be selected from Cpf1, FnCas12a, LbCas12a, AsCas12a, LbCas12a, TsCas12a, SaCas12a, Pb2Cas12a, PgCas12a, MiCas12a, Mb2Cas12a, M1b3Cas12a, Lb4Cas12a, Lb5Cas12a, FbCas12a, CpbCas12a, CrbCas12a, CMaCas12a, BsCas12a, BfCas12a, BoCas12a. In some embodiments, the CRISPR/Cas protein may be derived from a bacteria or has one or more components derived from a bacteria, and wherein the one or more components may optionally be derived from different bacteria. The bacteria origin of the CRISPR/Cas protein of each of the epigenetic modulators may be selected from Streptococcus pyogenes, Streptococcus thermophilus, Streptococcus sp., Staphylococcus aureus, Nocardiopsis dassonvillei, Streptomyces pristinac spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Bacillus niameyensis, Bacillus okhensis, Capnocytophaga canis, Chryseobacterium gallinarum, Coriobacterium_glomerans_PW2, Dechloromonas denitrificans, Enterococcus cecorun, Enterococcus faccium, Enterococcus italicus, Eubacterium dolichum, Eubacterium sp., Eggerthella sp. YY7918, Exiguobacterium sibiricum, Flavobacaterium frigidarium, Facklamia hominis, Finegoldia_magna_ATCC_29328, Kingella kingae, Lactobacillus_rhamnosus LOCK900, Lactobacillus delbrueckii, Lactobacillus salivarius. Lactobacillus sp., Microscilla marina, Mycoplasma_gallisepticum_CA06, Neissena meningitidis, Ornithobacterium rhinotracheale, Burkholderiales bacterium, Polaromonas naphthaienivorans, Polaromonas sp., Pediococcus acidilactici, Prevotella histicola, Parabacteroides sp., Streptococcus_agalactiae NEM316, Streptococcus_dysgalactiac_subsp._equisimilis_AC-2713, Streptococcus equinus, Streptococcus galiolyticus, Streptococcus gordonii, Streptococcus mutans GS-5, Streptococcus macedonicus, Streptococcus ratti, Streptococcus_salivarius_JIM8777, Streptococcus sinensis, Streptococcus suis D9. Streptococcus thermophilus LMG 18311, Tissierellia bacterium KA00581, Treponema denticola ATCC 35405, Treponema putidum, Turicibacter sp., Veillonella parvula ATCC 17745, Weeksella virosa, Streptococcus equi, Streptococcus agalactiae, Lactobacillus animnalis KCTC 3501, Listeria monocytogenes, Lachnospiraceae bacterium ND2006, Acidaminococcus sp. BV3TL6, Helcococcus kunzii, Prevotella ibumii, Prevotella bryantii B14, Compost_meta-_(Ga0079224_100045232_-CRISPR associated_protein, _Csn1_family_CDS_translation_Compost_meta, Geyser Hotspring_Yellowstone_Ga0078972_1022257_-CRISPR-associated_protein, _Csn1_family_CDS_translation_Community_metagenome, Geyser-Hotspring_Yellowstone_(Ga0078972_1010018-_CRISPR-associated_protein, _Csn1_family_CDS_translation_Community_metagenome, Crocosphaera watsonii, Cyanothece sp., Microcystis aeruginosa, Pseudomonas aeurginosa, Synechococcus sp., Acetohalobium arabaticum, Ammonifex degensii, Caldicelulosinuptor becscii, Candidatus Desulforudis, Clostridium botulinum, Clostridium difficile, Clostridium Tyrobutynicum, Clostridium beijerinckii, Clostridium perfringens, Clostridium autoethanogenum, Finegoldia magna, Natranaerobius thermophilus, Methanococcus maripaludis, Pelotomaculum thennopropionicumn, Acidithiobacillus caldus, Lactobacillus crispatus, Acidithiobacillus ferrooxidans, Acidaninococcus intestine RyC-MR95, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Streptococcus thermophilus, Lactococcus lactis, Staphylococcus epidennidis Anabaena vaniabilis, Nodularia spurnigena, Nostoc sp., Arthrospira maxima, Arthrospira platensis, Arthrospira sp., Lyngbya sp., Microcoleus chthonoplastes, Oscillartoria sp., Petrotoga mobilis, Thermosipho africanus, Clostridium acetobutylicum, Synechococcus elongatus UTEX 2973, Actinoplanes sp., B. subtilis, Corynebacterium glutamicum, Streptomyces sp. Clostridium difficile, Clostridium saccharoperbutylacetonicum N1-4, Acaryochloris marina, Leptotrichia shahii, and Francisella novicida.

The CRISPR/Cas protein may be derived from a virus, e.g., a phage virus, e.g., a bacteriophage, e.g., a Biggievirus or has one or more components derived from a virus, e.g., a phage virus, e.g., a bacteriophage, e.g., a Biggievirus and wherein the one or more components may optionally be derived from different virus.

In some embodiments, the CRISPR/Cas domain comprises a modified form of a wild-type Cas protein. The modified from of the wild-type Cas protein can comprise one or more amino acid changes (e.g., deletion, insertion, or substitution). In some embodiments, the endonuclease domain may comprise one or more amino acid substitutions as compared to a wild-type endonuclease domain. In some embodiments, the CRISPR/Cas domain comprises an endonuclease domain that has modified or reduced nuclease activity as compared to a wild-type protein. For example, the endonuclease domain can have less than 90%, less than 80%, less than 70%, less than 60%, less than 50%, less than 40%, less than 30%, less than 20%, less than 10%, less than 5%, or less than 1% nuclease activity of the wild-type Cas protein. In some embodiments, the CRISPR/Cas domain comprises a catalytically inactive CRISPR/Cas protein (e.g., dCas9) or a CRISPR/Cas protein with substantially reduced nuclease activity compared to a wild-type CRISPR/Cas protein. Many catalytically inactive CRISPR/Cas proteins are known in the art. A catalytically inactive CRISPR/Cas protein or a CRISPR/Cas protein that has reduced DNA cleavage activity with respect to both strands of a double-stranded target DNA can result from deletion or mutation of all of the nuclease domains of a CRISPR/Cas protein (e.g., both RuvC and HNH nuclease domains in a Cas9 protein: RuvC nuclease domain in a Cpf1 protein). For example, a catalytically inactive S. pyogenes Cas9 can result from a D10A (aspartate to alanine at position 10) mutation in the RuvC domain and H939A (histidine to alanine at amino acid position 839) or H840A (histidine to alanine at amino acid position 840) in the HNH domain. A catalytically inactive CRISPR/Cas protein (e.g., dCas, dCas9) can bind to a target polynucleotide but may not cleave the target polynucleotide. Examples of mutations in Cas9 include but are not limited to D10A, D11A, D16A, D17A, H557A, H558A, H588A, N611A, N612A, H589A, H820A, H821A, D839A, H840A, N863A, N864A, D917, D918A, H969A, H970A, E993A, E994A, N995A, N996A, E1006A, E1007A, D1255A, D1256A, or any combination thereof. In some embodiments, a spCas9 mutation include e.g., D10A/H820A, D10A, D10A/D839A/1840A, and D01A/D839A/H840A/N863A or any combination thereof.

In some embodiments, the CRISPR/Cas domain comprises a CRISPR/Cas domain that has single strand DNA cleavage activity when contacted with a double stranded DNA sequence. In some embodiments, the CRISPR/Cas domain comprises a CRISPR/Cas domain (i.e., a nickase) that can generate a single-strand break but not a double-strand break. Many CRISPR/Cas nickases are known in the art. A CRISPR/Cas nickase can result from deletion or mutation of one of the nuclease domains in a Cas protein comprising at least two nuclease domains (e.g., Cas9). For example, an S. pyogenes Cas9 nickase can result from a D10A (aspartate to alanine at position 10) mutation in the RuvC domain or a H839A (histidine to alanine at amino acid position 839) or H840A (histidine to alanine at amino acid position 840) mutation in the HNH domain.

In some embodiments, a Cas protein described herein is a mature Cas protein, e.g., lacking a N terminal methionine. A Cas protein can be a chimeric Cas protein that is fused to other proteins or polypeptides. A Cas protein can be a chimera of various Cas proteins, for example, comprising domains of Cas proteins from different organisms. In some embodiments, a Cas9 is a chimeric Cas9, e.g., modified Cas9, e.g., synthetic RNA-guided nucleases (sRGNs), e.g., modified by DNA family shuffling, e.g., sRGN3.1, sRGN3.3. In some embodiments, the DNA family shuffling comprises, fragmentation and reassembly of parental Cas9 genes, e.g. one or more of Cas9s from Staphylococcus hyicus (Shy), Staphylococcus lugdunensis (Slu), Staphylococcus microti (Smi) and Staphylococcus pasteuri (Spa).

PAM sequences: A target DNA sequence must generally be adjacent to a “protospacer adjacent motif” (“PAM”) that is specific for a given Cas domain; however, PAM sequences appear throughout a given genome. In some embodiments, the PAM is required for target binding of the Cas protein. The specific PAM sequence required for Cas domain recognition may depend on the specific type of the Cas domain. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In some embodiments, the PAM can be a 3′ PAM (i.e., located downstream of the 5′ end of the protospacer). In some embodiments, the Cas domain recognizes a canonical PAM, for example, a SpCas9 recognizes 5′-NGG-3′ PAM. In some embodiments, a Cas domain described herein has altered PAM specificity. In some embodiments, a Cas domain described herein may have one or mutations in a PAM recognition motif Examples of specific PAM sequences are provided in Table 3 below. As used in PAM sequences in Table 3 and consensus sequences of exemplary promoter elements in Table 1, “N” refers to any one of nucleotides A, G. C, and T, “R” refers to nucleotide A or G, “Y” refers to nucleotide C or T, “W” refers to nucleotide A or T, “K” refers to nucleotide G or T, “M” refers to nucleotide A or C, “B” refers to nucleotide C or C or T, “D” refers to nucleotide A or C or T. “H” refers to nucleotide A or C or T, and “V” refers to nucleotide A or C or G

TABLE 3
Exemplary PAM Sequences of CRISPR/Cas Proteins

| | | | SEQ |
|---|---|---|---|
| Organism / source | Abbreviation | PAM Sequence | Identifier |
| Acidaminococcus_intestini_ | Ain | NRTAW | A11 |
| RyC-MR95 | | | |
| Bacillus niameyensis | Bni | NNGTAMAY | A12 |
| Bacillus okhensis | Bok | NNHDAGGDNA | A13 |
| Capnocytophaga canis | Cca1 | BRTTTTT | A14 |
| Chryseobacterium | Cga | BRRTTTW | A15 |
| gallinarum | | | |
| Coriobacterium_glomerans | Cg1 | NCAARC | A16 |
| PW2 | | | |
| Compost_meta_- | Cme1 | NARNCCN | A17 |
| _Ga0079224_100045232_- | | | |
| _CRISPR- | | | |
| associated_protein,_Csn1_ | | | |
| family_CDS_translation_ | | | |
| Compost_meta | | | |
| Clostridium perfringens | Cpe | NNNNCVGAA | A18 |
| Dechloromonas | Dde | NNNNGYAA | A19 |
| denitrificans | | | |
| Enterococcus cecorum | Ece | NNGHWAAA | A20 |
| Eubacterium dolichum | Edo | TGARGNANY | A21 |
| Enterococcus faecium | Efa | BTGGDATNN | A22 |
| Enterococcus italicus | Eit | NGGGAHTNAN | A23 |
| Eggerthella_sp._YY7918 | Esp1 | NAAAG | A24 |
| Eubacterium sp. | Esp2 | NVARAACCN | A25 |
| Flavobacterium frigidarium | Ffr | NARATC | A26 |
| Facklamia hominis | Fho | NNGAAAN | A27 |
| Finegoldia_magna_ATCC_ | Fma | NNGAC | A28 |
| 29328 | | | |
| Geyser- | Ghy2 | NAANARCN | A29 |
| Hotspring_Yellowstone_ | | | |
| Ga0078972_1022257_- | | | |
| _CRISPR- | | | |
| associated_protein, Csn1_ | | | |
| family_CDS_translation_ | | | |
| Community_metagenome | | | |
| Geyser- | Ghy4 | YHHNGTH | A30 |
| Hotspring_Yellowstone_ | | | |
| Ga0078972_1010018_- | | | |
| _CRISPR- | | | |
| associated_protein,_Csn1_ | | | |
| family_CDS_translation_ | | | |
| Community_metagenome | | | |
| Kingella kingae | Kki | NNNNCTAA | A31 |
| Lactobacillus_rhamnosus_ | Lrh | NTNTAAWA | A32 |
| LOCK900 | | | |
| Lactobacillus sp. | Lsp1 | NNGWAAYT | A33 |
| Lactobacillus sp. | Lsp2 | NCAAHYBY | A34 |
| Mycoplasma_gallisepticum | Mga | NNGAD | A35 |
| CA06 | | | |
| Neisseria meningitidis | Nme2 | NHDTCCA | A36 |
| Neisseria sp. | Nsp | NCNNTCCN | A37 |
| Ornithobacterium | Orh | NNAAARG | A38 |
| rhinotracheale | | | |
| Pediococcus acidilactici | Pac | NHTAAAA | A39 |
| Prevotella histicola | Phi | NARGHWHAGNC | A40 |
| Parabacteroides sp. | Psp | NRATTTT | A41 |
| Streptococcus_agalactiae_ | Sag2 | NGGDAWT | A42 |
| NEM316 | | | |
| Streptococcus_dysgalactiae_ | Sdy | NGGNG | A43 |
| subsp._equisimilis_AC- | | | |
| 2713 | | | |
| Streptococcus equinus | Seq1 | NTAGANANN | A44 |
| Streptococcus_gallolyticus | Sga | NGGDAHT | A45 |
| subsp._gallolyticus_ ATCC_ | | | |
| 43143 | | | |
| Streptococcus_gordonii_str. | Sgo | NNAAAG | A46 |
| Challis substr. CH1 | | | |
| Streptococcus_mutans_GS- | Smu | NGGDT | A47 |
| 5 | | | |
| Streptococcus macedonicus | Sma2 | NHGYNANA | A48 |
| Streptococcus ratti | Sra | NGGDAGNN | A49 |
| Streptococcus_salivarius_ | Ssa | NNNAGAAA | A50 |
| JIM8777 | | | |
| Streptococcus sinensis | Ssi | NHAAAAA | A51 |
| Streptococcus_suis_D9 | Ssu | NHTAAAAA | A52 |
| Streptococcus_thermophilus | Sth1A | NHGYRAA | A53 |
| LMG 18311 | | | |
| Tissierellia bacterium | Tba | NNNATTT | A54 |
| KA00581 | | | |
| Treponema_denticola_ ATCC_ | Tde | NAAAAY | A55 |
| 35405 | | | |
| Treponema putidum | Tpu | NATAWNA | A56 |
| Turicibacter sp. | Tsp | NNAAACN | A57 |
| Veillonella parvula ATCC | Vpa | NATARCH | A58 |
| 17745 | | | |
| Weeksella virosa | Wvi | HHAAATD | A59 |
| Streptococcus pyogenes, | Spy, Sag1, | NGG | A60 |
| Streptococcus equi, | Seq2 | | |
| Streptococcus agalactiae | | | |
| Lactobacillus animalis | Lan | NRG | A61 |
| KCTC 3501 | | | |
| Listeria monocytogenes | Lmo | NBGG | A62 |
| Lachnospiraceae bacterium | Cpf1 | TTTV | A63 |
| ND2006, Acidaminococcus | | | |
| sp. BV3L6 | | | |
| Helcococcus kunzii | HkCas12a | YYV | A64 |
| Prevotella ihumii | PiCas 12a | KKYV | A65 |
| Francisella novicida | Fn3Cas12a | YTV | A66 |
| Prevotella bryantii B14 | Pb2Cas12a | NYTV | A67 |
| Biggievirus strain Mosll | CasPhi-2 | TBN | A68 |
| Oleiphilus sp. HI0009 | Cas12c1 | TG | A69 |

### Zinc Finger Domains

In some embodiments, a nucleic acid binding moiety may be or comprises a Zn finger domain. Zn finger proteins and methods for design and construction of fusion proteins are known to those of skill in the art. The Zn finger domain may comprise or consist essentially of or consist of 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 zinc fingers. Zn finger proteins and/or multi fingered Zn finger proteins may be linked together, e.g., as a fusion protein, using any suitable linker sequences. The Zn finger domain may include any combination of suitable linkers between the individual Zn finger proteins and/or multi-fingered Zn finger proteins of the Zn finger molecule.

The Zn finger domain of an epigenetic modulator may comprise a Zn finger molecule comprising an engineered zinc finger protein that binds (in a sequence-specific manner) to a DNA sequence in a target nucleic acid. Engineering methods include, but are not limited to, rational design and various types of selection. Rational design includes, for example, using databases comprising triplet (or quadruplet) nucleotide sequences and individual Zn finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,453,242 and 6,534,261, incorporated by reference herein in their entireties. Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as International Patent Publication Nos. WO 98/37186; WO 98/53057; WO 00/27878; and WO 01/88197 and GB 2,338,237. In addition, enhancement of binding specificity for zinc finger proteins has been described, for example, in International Patent Publication No. WO 02/077227.

In some cases, a Zn finger molecule may comprise a two-handed Zn finger protein. Two handed Zn finger proteins are those proteins in which two clusters of zinc finger proteins are separated by intervening amino acids so that the two Zn finger domains bind to two discontinuous target DNA sequences. An example of a two-handed type of zinc finger binding protein is SIP1, where a cluster of four zinc finger proteins is located at the amino terminus of the protein and a cluster of three Zn finger proteins is located at the carboxyl terminus (Remade et al. 1999). Each cluster of zinc fingers in these proteins is able to bind to a unique target sequence and the spacing between the two target sequences can comprise many nucleotides.

In some embodiments, the Zn finger domain comprises a ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZN7F677, ZNF596, ZNF214, ZNF37A, ZNF34, ZNF250, ZNF547, ZNF273, ZNF354A, ZNF82, ZNF224, ZNF33A, ZNF45, ZNF175, ZNF595, ZNF184, ZNF419, ZNF28-1, ZNF28-2, ZNF18, ZNF213, ZNF394, ZNF1, ZNF14, ZNF416, ZNF557, ZNF566, ZNF729, ZIM2, ZNF254, ZNF764, ZNF7T85, ZNF10 (KOX1), ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN582, ZN540, ZN75D, ZN555, ZN658, ZN684, ZN829, ZN582, ZN112, ZN716, ZN350, ZN480, ZN416, ZNF92, ZN100, ZN736, ZNF74, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, ZNF12, ZN169, ZN433, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z5873, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, ZN490, ZN805, ZN80B, ZN763, ZN285, ZNF85, ZN223, ZNF90, ZN557, ZN425, ZN229, ZN606, ZN155, ZN222, ZN442. ZNF91, ZN135, ZN778, ZN534, ZN586, ZN567, ZN440, ZN583, ZN441, ZNF43, ZNF589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, ZN101, ZN783, ZN417, ZN182, ZN823, ZN177, ZN197, ZN717, ZN669, ZN256, ZN251, ZN562, ZN461, Z324A, ZN766, ZN473, ZN496, ZN597, ZN274, ZN783, ZN840, ZN777, ZN212, ZN214, ZN764, ZNF17, ZN282, ZNF81, or ZN298 domain.

### TAL Domains

In some embodiments, a nucleic acid binding moiety is or comprises a TAL domain. A TAL domain is derived from a TAL effector molecule that specifically binds a DNA sequence. TAL effectors typically comprise a plurality of TAL effector domains or fragments thereof, and optionally one or more additional portions of naturally occurring TAL effectors (e.g., N- and/or C-terminal of the plurality of TAL effector domains). More than 113 TAL effector sequences are known to date. Non-limiting examples of TAL effectors from Xanthomonas include Hax2, Hax3, Hax4, AvrXa7, AvrXalO and AvrBs3. Many TAL domains are known to those of skill in the art and are commercially available.

TAL effectors comprise a central repeat domain of tandemly arranged repeats (the repeat-variable di-residues, RVD domain) that determine the specific binding of TAL effectors. These repeats are typically 33 or 34 amino acids. Different TAL effectors may have a different number of repeats (typically ranging from 1.5 to 33.5 repeats) and a different order of their repeats. The C-terminal repeat is usually shorter in length (e.g., about 20 amino acids) and is generally referred to as a “half-repeat”, Each repeat of the TAL effector generally correlates to one base-pair in the target DNA sequence with different repeat types exhibiting different base-pair specificity. A smaller number of repeats generally results in weaker protein-DNA interactions. A number of 6.5 repeats in a TAL effector has been shown to be sufficient to activate transcription of a reporter gene (Scholze et ab, 2010).

Many variations between repeats occur at amino acid positions 12 and 13, which have been termed “hypervariable” and are responsible for the specificity of the interaction with the target DNA promoter sequence, as shown in Table 4 listing exemplary repeat variable di-residues (RVD) and their corresponding nucleic acid base targets.

TABLE 4
RVDs and Nucleic Acid Base Specificity

| Target | Possible RVD Amino Acid Combinations | | | | | | | | | | | | |
|---|---|---|---|---|---|---|---|---|---|---|---|---|---|
| A | NI | NN | CI | HI | KI | | | | | | | | |
| G | NN | GN | SN | VN | LN | DN | QN | EN | HN | RH | NK | AN | FN |
| C | HD | RD | KD | ND | AD | | | | | | | | |
| T | NG | HG | VG | IG | EG | MG | YG | AA | EP | VA | QG | KG | RG |

The RVD NK has also been shown to target G. Many target sites of TAL effectors also include a T flanking the 5 base targeted by the first repeat.

In some embodiments, the TAL domain described herein may be derived from a TAL effector from any bacterial species (e.g., Xanthomonas species such as the African strain of Xanthomonas oryzae pv. Oryzae (Yu et al. 2011), Xanthomonas campestris pv. raphani strain 756C and Xanthomonas oryzae pv. oryzzco/a strain BLS256 (Bogdanove et al. 2011). In some embodiments, the TAL domain comprises an RVD domain as well as flanking sequence(s) (sequences on the N-terminal and/or C-terminal side of the RVD domain) also from the naturally occurring TAL effector. It may comprise more or fewer repeats than the RVD of the naturally occurring TAL effector. The TAL domain can be designed to target a given nucleic acid sequence based on Table 4 and other nucleic acid base specificities known in the art. The TAL domain of an epigenetic modulator can comprise a number of TAL effector domains (e.g., repeats (monomers or modules)) selected based on the desired binding site to a target nucleic acid. TAL effector domains, e.g., repeats, may be removed or added in order to suit a specific binding target sequence. In some cases, the TAL domain of an epigenetic modulator may comprise between 6.5 and 33.5 TAL effector domains, e.g., repeats. In some cases, TAL domain of an epigenetic modulator may comprise between 8 and 33.5 TAL effector domains, between 10 and 25 TAL effector domains, or between 10 and 14 TAL effector domains. In some cases, the TAL domain of an epigenetic modulator may comprise TAL effector domains that correspond to a perfect match to the DNA target sequence. In some cases, the TAL domain of an epigenetic modulator may comprise a mismatch between a repeat and a target base-pair in the target nucleic acid as along as it allows for the function of the epigenetic modulator comprising the TAL effector molecule. In some cases, the TAL domain of an epigenetic modulator comprises no more than 7 mismatches, 6 mismatches, 5 mismatches, 4 mismatches, 3 mismatches, 2 mismatches, or 1 mismatch, and optionally no mismatch, with the target DNA sequence. In general, TAL binding is inversely correlated with the number of mismatches. Without wishing to be bound by theory, in general the smaller the number of TAL effector domains in the TAL domain of the epigenetic modulator, the smaller the number of mismatches will be tolerated and still allow for the function of the epigenetic modulator comprising the TAL domain. The binding affinity of the TAL domain to the target nucleic acid is thought to depend on the sum of matching repeat-DNA combinations. For example, TAL effector molecules having 25 TAL effector domains or more may be able to tolerate up to 7 mismatches.

In addition to the TAL effector domains, the TAL domain of an epigenetic modulator may comprise additional sequences derived from a naturally occurring TAL effector. The length of the C-terminal and/or N-terminal sequence(s) included on each side of the TAL effector domain portion of the TA L domain can vary and be selected by one skilled in the art. For example, a number of C-terminal and N-terminal truncation mutants in Hax3 derived TAL-effector based proteins have been characterized (Zhang et al. 2011) and key elements have been identified that contribute to optimal binding to the target sequence and activation of transcription. Transcriptional activity was generally found to inversely correlate with the length of N-terminus. On the C-terminus side, an important element for DNA binding residues was identified within the first 68 amino acids of the Flax 3 sequence. Accordingly, in some cases, the first 68 amino acids on the C-terminal side of the TAL effector domains of the naturally occurring TAL effector may be included in the TAL domain of the epigenetic modulator. In some cases, a TAL domain in an epigenetic modulator comprises 1) one or more TAL effector domains derived from a naturally occurring TAL effector; 2) at least 70, 8, 90, 100, 110, 120, 130, 140, 150, 170, 180, 190, 200, 220, 230, 240, 250, 260, 270, 280 or more amino acids from the naturally occurring TAL effector on the N-terminal side of the TAL effector domains, and/or 3) at least 68, 80, 90, 100, 110, 120, 130, 140, 150, 170, 180, 190, 200, 220, 230, 240, 250, 260 or more amino acids from the naturally occurring TAL effector on the C-terminal side of the TAL effector domains.

It is possible to modify the repeats to target specific DNA sequences. In some embodiments, the TAL effector domain of an epigenetic modulator can be engineered to carry the epigenetic modulator to desired target sites.

### OMEGA System Domains

In some embodiments, a nucleic acid binding moiety may be or comprise a domain from an obligate mobile element-guided activity (OMEGA) system. The OMEGA domain can comprise an RNA-programmable nuclease domain. In some cases, the OMEGA domain can comprise a distinct transposon-encoded protein domain, for example, an IscB domain, an IsrB domain, an IshB domain, or an TnpB domain. The OMEGA domain can be an ancestor or a variant of an ancestor of a CRISPR nuclease domain, for example, a Cas9 domain or a Cas12 domain. An IscB domain or an TnpB domain can be encoded in family of IS200/IS605 transposons. The OMEGA domain can comprise a nuclease domain. In some cases, the OMEGA domain comprises a RuvC domain or an HNH domain. In some cases, the OMEGA domain comprises a RuvC domain and an HNH domain. In other cases, the OMEGA domain can comprise an HNH domain but no RuvC domain. The OMEGA domain can further comprise a PLMP domain. In some cases, the OMEGA domain is catalytically active. The OMEGA domain can, for example, comprise nickase activity. The OMEGA domain can be mutated to be deficient in nuclease activity. In some cases, the OMEGA domain is catalytically inactive.

In some cases, the OMEGA domain can comprise RNA-guided activity. In some cases, an OMEGA domain can comprise an RNA-guided nuclease. An OMEGA domain can be capable of specifically interacting with or binding to a specific noncoding RNA, for example, an ωRNA. The noncoding RNA can be configured to recruit the OMEGA domain to a specific target sequence, for example, by hybridization of a segment of the noncoding RNA to the target sequence. In some cases, hybridization of the segment of the nonRNA to the target sequence triggers the OMEGA domain to activate its nuclease domain and carry out double-stranded DNA cutting or a single-stranded DNA nick at the target sequence. In some cases, the noncoding; RNA that interacts with the OMEGA domain comprises a CRISPR repeat sequence or a sequence from a CRISPR array. In some cases, the OMEGA domain is associated with a CRISPR array. In some cases, the OMEGA domain is capable of associating with a particular target adjacent motif (TAM). The OMEGA domain may require binding to the TAM in order to activate its RNA-guided activity.

In some embodiments, an OMEGA domain is a part of an epigenetic modulator described elsewhere herein. In some embodiments, an OMEGA domain is a part of a blocking reagent described elsewhere herein. An OMEGA domain can be the nucleic acid binding domain of an epigenetic modulator. An OMEGA domain can be coupled to an effector moiety described elsewhere herein, for example, as a fusion protein. Alternatively, an OMEGA domain can be the nucleic acid binding domain of a blocking reagent described elsewhere herein.

### Fanzor Domain

In some embodiments, a nucleic acid binding moiety may be or comprise a Fanzor domain. The Fanzor domain can comprise an RNA-programmable nuclease domain. In some cases, the Fanzor domain is derived from a eukaryotic cell or an engineered variant thereof. The Fanzor domain can be derived from a metazoan, fungus, choanoflagellate, algae, rhodophyta, a unicellular eukaryote, plant, or animal. In further cases, the Fanzor domain is derived from a virus or an engineered variant thereof. For example, the Fanzor domain can be derived from Phycodnaviridae, Ascoviridae, or Mimiviridae. In some cases, the Fanzor domain is derived from the Acanthamoeba polyphaga mimivirus, Mercenaria, Dreissena polymorpha, Batillaria attroamentaria, Klebsormidium nitens, or Chlamydomonas reinhardtii. The Fanzor domain can comprise a homolog of a TnpB domain. A Fanzor domain can be capable of associating with a eukaryotic transposase. In some cases, a Fanzor domain is capable of associating with a LINE, CMC, Crypton, Mariner/Tc1, hAT, IS607, EnSpm, Sola, or Helitron transposon. The Fanzor domain can comprise a nuclease domain. In some cases, the Fanzor domain comprises a RuvC domain. The Fanzor domain can further comprise a WED domain. In some cases, the Fanzor domain is catalytically active. The Fanzor domain can, for example, comprise nickase activity. The Fanzor domain can be mutated to be deficient in nuclease activity. In some cases, the Fanzor domain is catalytically inactive.

In some cases, the Fanzor domain can comprise RNA-guided activity. In some cases, an Fanzor domain can comprise an RNA-guided nuclease. A Fanzor domain can be capable of specifically interacting with or binding to a specific noncoding RNA, for example, an ⋅RNA. The noncoding RNA can be configured to recruit the Fanzor domain to a specific target sequence, for example, by hybridization of a segment of the noncoding RNA to the target sequence. In some cases, hybridization of the segment of the nonRNA to the target sequence triggers the Fanzor domain to activate its nuclease domain. In some cases, an activated Fanzor domain carries out double-stranded DNA cutting or a single-stranded DNA nick at the target sequence. In some cases, the Fanzor domain is capable of associating with a particular target adjacent motif (TAM). The Fanzor domain may require binding to the TAM in order to activate its RNA-guided activity. The Fanzor domain can be smaller in size compared to a CRISPR Cas9 protein or a CRISPR Cas12 protein.

In some embodiments, a Fanzor domain is a part of an epigenetic modulator described elsewhere herein. In some embodiments, a Fanzor domain is a part of a blocking reagent described elsewhere herein. A Fanzor domain can be the nucleic acid binding domain of an epigenetic modulator. A Fanzor domain can be coupled to an effector moiety described elsewhere herein, for example, as a fusion protein. Alternatively, a Fanzor domain can be the nucleic acid binding domain of a blocking reagent described elsewhere herein.

### Vectors

The present disclosure is further directed, in part, to vectors, e.g., a viral vector and/or a non-viral vector. An epigenetic modulator or a blocking reagent described herein can be delivered via a vector into a cell via electroporation, chemical transformation, nucleofection, viral transduction, viral transfection, or other similar techniques.

In some embodiments, the vector is a viral vector. Examples of viral vectors include expression vectors, replication vectors, probe generation vectors, and sequencing vectors. In general, a suitable vector contains an origin of replication functional in at least one organism, a promoter sequence, convenient restriction endonuclease sites, and one or more selectable markers. An expression vector may be used to express natural or synthetic nucleic acids by operably linking a nucleic acid encoding the gene of interest to a promoter, Vectors can be suitable for replication and integration in eukaryotes. Typical cloning vectors contain transcription and translation terminators, initiation sequences. and promoters useful for expression of the desired nucleic acid sequence. Viral vectors, including those derived from retroviruses such as lentivirus, are suitable tools to achieve long-term gene transfer since they allow long-term, stable integration of a transgene and its propagation in daughter cells. An expression vector may be provided to a cell in the form of a viral vector. Viral vector technology is well known in the art and described in a variety of virology and molecular biology manuals. Viruses, which are useful as vectors include, but are not limited to, retroviruses, adenoviruses, adeno-associated viruses (AAV), herpes viruses, and lentiviruses.

An AAV can be AAV1, AAV2, AAV4, AAV5, AAV6, AAV8, AAV9, AAV 10 or any combination thereof. One can select the type of AAV with regard to the cells to be targeted, e.g., one can select AAV serotypes 1, 2, 5 or a hybrid capsid AAV, AAV2, AAV5 or any combination thereof for targeting brain or neuronal cells; and one can select AAV4 for targeting cardiac tis-sue. AAV8 is useful for delivery to the liver.

In certain instances, recombinant AAV (rAAV) may be used. rAAVs utilizes the cis-acting 145-bp ITRs to flank vector transgene cassettes, providing up to 4.5 kb for packaging of foreign DNA.

In some embodiments, a vector comprises an expression cassette comprising the nucleic acid encoding a protein or functional RNA. In some embodiments, the protein or functional RNA in the expression cassette is operatively linked to a promoter sequence that controls the expression of the protein or functional RNA, The present disclosure should not be interpreted to be limited to use of any particular promoter or category of promoters. In some embodiments, the promoter may be an inducible promoter that is capable of turning on expression of a polynucleotide sequence to which it is operatively linked, when such expression is desired. In some embodiments, the inducible promoter is capable of turning off expression when expression is not desired. Examples of inducible promoters include, but are not limited to a metallothionine promoter, a glucocorticoid promoter, a progesterone promoter, and a tetracycline promoter.

In some embodiments, the vector comprising an expression cassette may contain a selectable marker gene (e.g., antibiotic resistance gene) or a reporter gene (e.g., luciferase, beta-galactosidase, green fluorescent protein gene) to facilitate identification and selection of cells containing the vector. Suitable expression systems are well known to one of skill in the art and may be prepared using known techniques or obtained commercially.

In some embodiments, the present disclosure provides a composition of a vector or vector set encoding an epigenetic modulator, a blocking reagent, a guide RNA, or any polypeptide or nucleic acid described elsewhere herein. In some such embodiments, provided vectors may be or include DNA, RNA, e.g., mRNA, or any other nucleic acid moiety or entity as described herein, and may be prepared by any technology described herein or otherwise available in the art (e.g., synthesis, cloning, amplification, in vitro or in vivo transcription, etc.). In some embodiments, provided nucleic acids that encode an epigenetic modulator, a blocking reagent, a guide RNA, or a nucleic acid in a guided epigenetic editing composition described elsewhere herein may be operationally associated with one or more replication, integration, and/or expression signals appropriate and/or sufficient to achieve integration, replication, and/or expression of the provided nucleic acid in a system of interest (e.g., in a particular cell, tissue, organism, etc.).

In some embodiments, the vector is a non-viral vector, e.g., liposome, exosome, lipid nanoparticle. In some embodiments, the vector may be selected from a lipid nanoparticle, a liposome, an exosome, and a micro vesicle. In some embodiments, the viral vector may be derived from an adenovirus, a retrovirus, an adeno-associated virus, a vaccinia virus, a lentivirus, a phage virus, a herpes simplex virus, or a polio virus. In some embodiments, the lipid nanoparticle may comprise an ionizable lipid. In some embodiments, the lipid nanoparticle further comprises one or more of neutral lipids, ionizable amine-containing lipids, biodegradable alkyne lipids, steroids, phospholipids, polyunsaturated lipids, structural lipids (e.g., sterols), PEG, cholesterol, or polymer conjugated lipids.

In some embodiments, the vector may be provided as a component of a reaction mixture. In some embodiments, the vector may be provided as a component of a composition comprising the vector and a pharmaceutically acceptable carrier. In some embodiments, the vector may be provided as a component of a culture comprising a cell. In some embodiments, the vector may be provided as a component of a production vector.

### Software, Systems, and Devices

In some aspects, provided herein are systems and devices that may be used to perform the methods described herein or a portion of such methods. Also provided is a non-transitory computer-readable storage media, which may store instructions or programs which, when executed by one or more processors, may cause the system or device to perform the methods or portions of the methods described herein. In some embodiments, the non-transitory computer-readable storage media comprise one or more programs for execution by one or more processors of a device, the one or more programs including instructions which, when executed by the one or more processors, cause the device or system to sequence a DNA molecule to provide a plurality of sequencing reads, assemble a plurality of contigs from a plurality of sequence reads, identify contigs as being associated with different cells in a cell population, obtain an epigenetic profile for a cell in a cell population, and/or determine a differential between an obtained epigenetic profile and a target epigenetic profile,

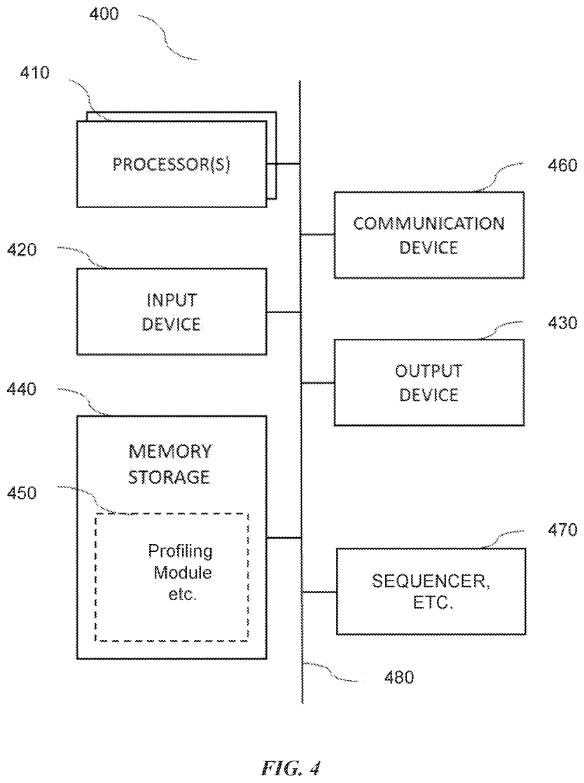

FIG. 4 illustrates an example of a computing device or system in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more processor(s) 410, input devices 420, output devices 430, memory or storage devices 440, communication devices 460, and a profiling data generation device (e.g., a nucleic acid sequencer) 470. Software 450 residing in memory or storage device 440 may comprise, e.g., an operating system as well as software for executing; the methods described herein. Input device 420 and output device 430 can generally correspond to those described herein, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage (e.g., an electrical, magnetic or optical memory including a RAM (volatile and non-volatile), cache, hard drive, or removable storage disk). Communication device 460 can include any suitable device capable of transmitting and receiving signals over a network, such as a network interface chip or device. The components of the computer can be connected in any suitable manner, such as via a wired media (e.g., a physical system bus 480, Ethernet connection, or any other wire transfer technology) or wirelessly (e.g., Bluetooth®, Wi-Fi®, or any other wireless technology).

Software module 450, which can be stored as executable instructions in storage 440 and executed by processor(s) 410, can include, for example, an operating system and/or the processes that embody the functionality of the methods of the present disclosure.

Software module 450 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 440, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

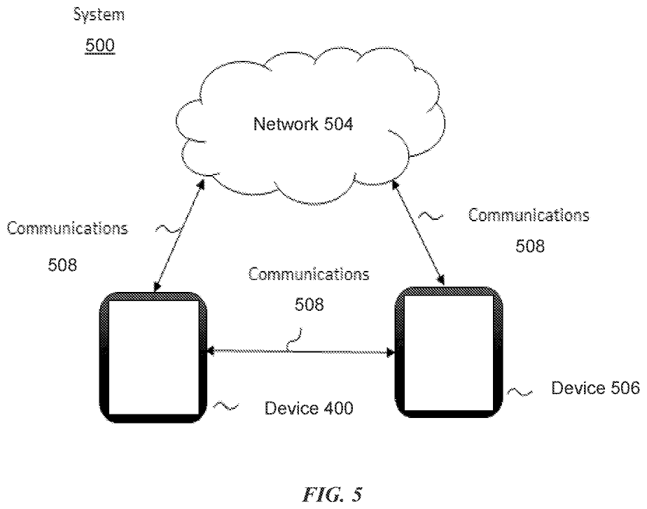

Device 400 may be connected to a network (e.g., network 504, as shown in FIG. 5 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can be implemented using any operating system, e.g., an operating system suitable for operating on the network. Software module 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 410.

Device 400 can further include, for example, a nucleic acid sequencer 470, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000, and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 5 illustrates an example of a computing system in accordance with one embodiment. In computing system 500, device 400 (e.g., as described above and illustrated in FIG. 4) is connected to network 504, which is also connected to device 506, In some embodiments, device 506 is a sequencer. Exemplary sequencers can include, without limitation, Roche/454's Genome Sequencer (GS) FLX System. Illumina/Solexa's Genome Analyzer (GA), Illumina'S HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 400 and 506 may communicate, e.g., using suitable communication interfaces via network 504, such as a Local Area Network (LAN), Virtual Private Network (VPN), or the Internet. In some embodiments, network 504 can be, for example, the Internet, an intranet, a virtual private network, a cloud network, a wired network, or a wireless network. Devices 400 and 506 may communicate, in part or in whole, via wireless or hardwired communications, such as Ethernet, IEEE 802.11b wireless, or the like. Additionally, devices 400 and 506 may communicate, e.g., using suitable communication interfaces, via a second network, such as a mobile/cellular network. Communication between devices 400 and 506 may further include or communicate with various servers such as a mail server, mobile server, media server, telephone server, and the like. In some embodiments, devices 400 and 506 can communicate directly (instead of, or in addition to, communicating via network 504), e.g., via wireless or hardwired communications, such as Ethernet, IEEE 802.11b wireless, or the like. In some embodiments, devices 400 and 506 communicate via communications 508, which can be a direct connection or can occur via a network (e.g., network 504).

One or all of devices 400 and 506 generally include logic (e.g., http web server logic) or are programmed to format data, accessed from local or remote databases or other sources of data and content, for providing and/or receiving information via network 504 according to various examples described herein.

### EXAMPLES

These examples are provided for illustrative purposes only and not to limit the scope of the claims provided herein.

### Example 1: Generating Epigenetic Maps Based on Unsupervised Clustering of Epigenetic States Using Long Read Sequencing

This example shows a method of generating epigenetic maps that depict methylation patterns in DNA from methylation sequence data using long read sequencing. Unsupervised clustering scheme was developed to identify epigenetic states on a whole genome and gene-level bases, using long read sequencing with methylation calling. Oxford Nanopore Technologies (ONT) was used to generate sequencing reads from CD8+ T-cells, isolated from three normal, healthy donors. All *.bam files were merged into one *.barn file to maximize coverage for this analysis.

Unsupervised clustering analysis was performed with the *.ban file, First, the region of interest (ROT) was selected. Given a set of coordinates spanning a genomic region (e.g., a gene), all fragments that span that region and the methylation status of any contained CpGs was extracted. All regions from the *.barn files were annotated as genes in the Gencode.v42 database including promoter regions (defined as 1 kb upstream as determined by strand annotation) https://ftp.ebi.ac.uk/pub/databases/gencode/Gencode human/release_42/gencode.v42.basic.ann otation.gtf.gz).

Next, for a given ROI, a distance matrix which contains a distance measure between all fragments that span that gene was computed. Each fragment was a vector of binary values corresponding to CpGs with either methylated (1) or unmethylated (0) values. Various distance metrics exist for computing the distance between two binary-valued vectors including Hamming, Random Forest and Simple Matching. In this implementation, Simple Matching, which evaluates the number of CpGs that match (e.g., both unmethylated or both methylated) and normalizes to the total number of comparable (i.e., CpGs) in the ROI was used.

With the commuted distance matrix, various fragments were grouped (clustered) to optimize an inter-cluster metric (e.g., minimize inter-cluster average distance) and an intra-cluster metric (e.g., maximize the distance between the two closest residents of two separate clusters). The two most common methods for clustering are hierarchical and k-means clustering. In this approach, hierarchical clustering was performed. Specifically, an agglomerative, hierarchical clustering with complete linkage (see: https://en.wikipedia.org/wiki/Complete-linkage clustering) was used.

Finally, the optimal number of clusters was determined. Common methods for determining the appropriate number of clusters include the Elbow Method, Silhouette, and the Gap Statistic. The appropriate number of clusters was determined by computing a figure of merit (FOM) while varying the number of clusters and selecting an optimal cluster number derived from the graph of the FOM vs. clusters (e.g., the elbow, maximum, etc.). Here, a version of the Gap Statistic was used.

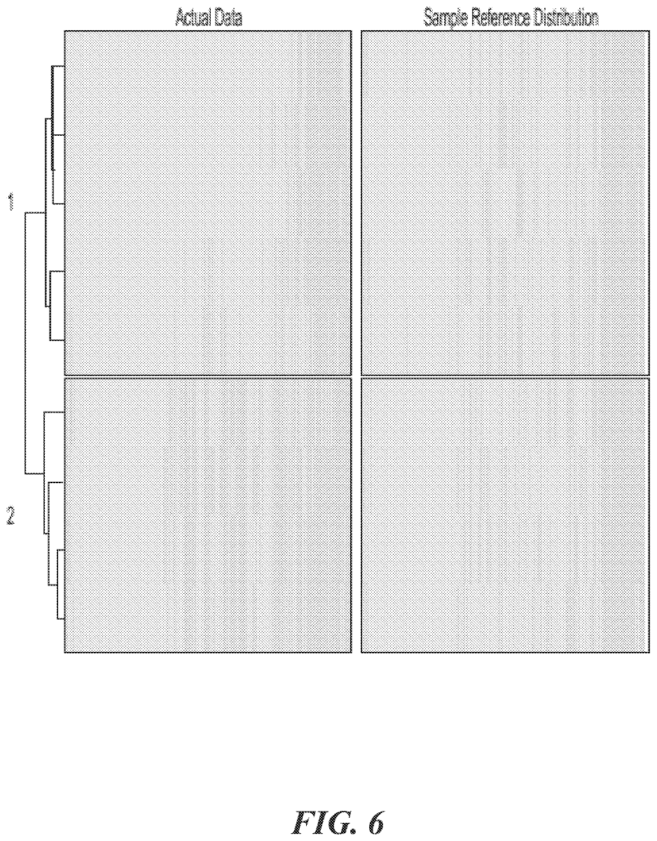

The Gap Statistic provides a method to evaluate the correct number of clusters by comparing the dispersion of inter-cluster distances to that obtained using a reference null distribution in which all samples are equidistant from one another (i.e., there should only be 1 cluster for the null hypothesis). To generate the correct reference null distribution, for each CpG, a state (1 or 0) from the distribution of fragments that span that CpG was randomly sampled. The resultant reference null data set eliminated the dependency structure of the actual data by ensuring all features (i.e., CpCs) were independent of one another. As shown in FIG. 6, actual and reference null data sets for TCF7 was compared. The columns represent CpGs in TCF7, and the rows represent individual fragments spanning TCF7.

This process was repeated multiple times (e.g., 50 times) to generate many reference null distributions. For each reference null distribution, a dispersion FOM (log(W)) was calculated. This was repeated for varying cluster number (up to a maximum determined by the number of fragments for that gene), The mean of the reference distribution FOM for each cluster number was compared to that obtained from the actual data and the Gap Statistic was calculated. Further, the standard error of the reference null FOM for each cluster number to as a means to assess the impact of random sampling on a given FOM to another was used.

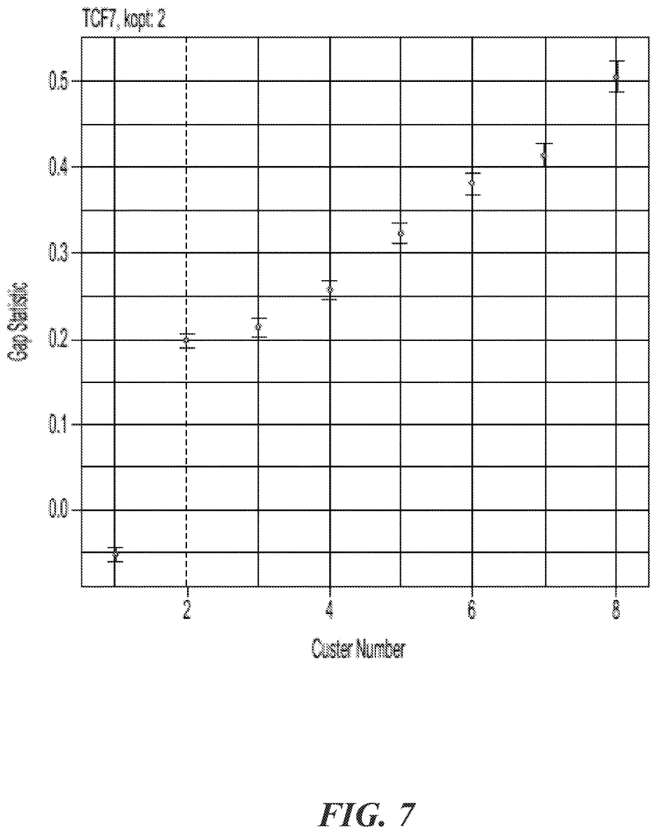

In this example, the smallest cluster number (k) that satisfies Gap(k)>=Gap(k+1)-3*SE(k+1) was selected. This enabled a statistical approach to selecting the appropriate number of clusters based on the underlying data distributions. As shown in FIG. 7, the dotted line, representing the optimal number of clusters for TCF7 (e.g., two clusters), was generated to satisfy Gap(k)>=Gap(k+1)−3*SE(k+1).

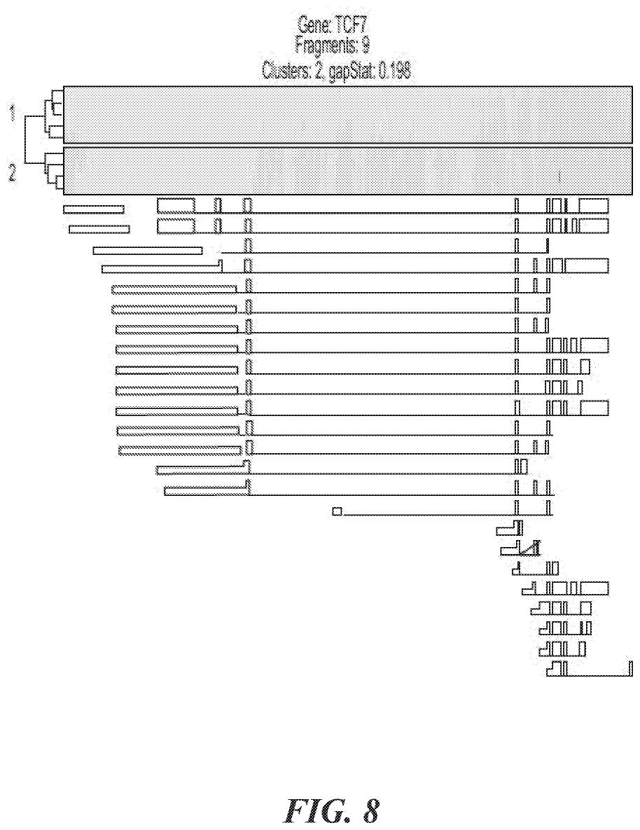

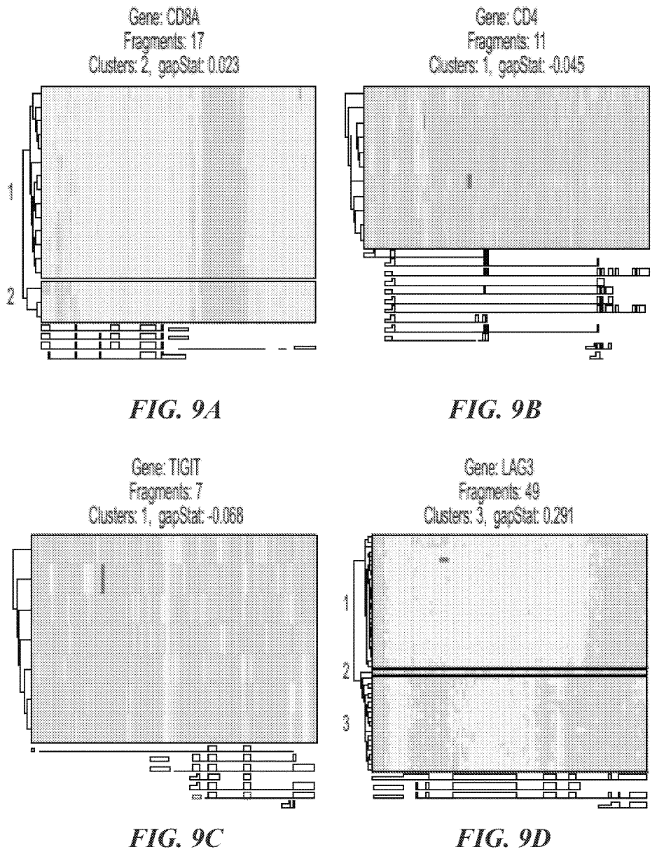

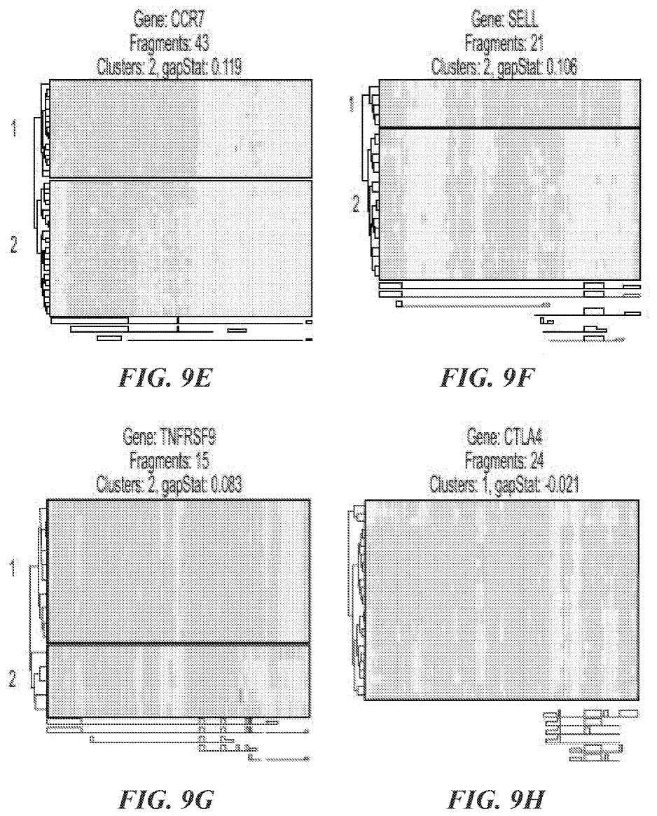

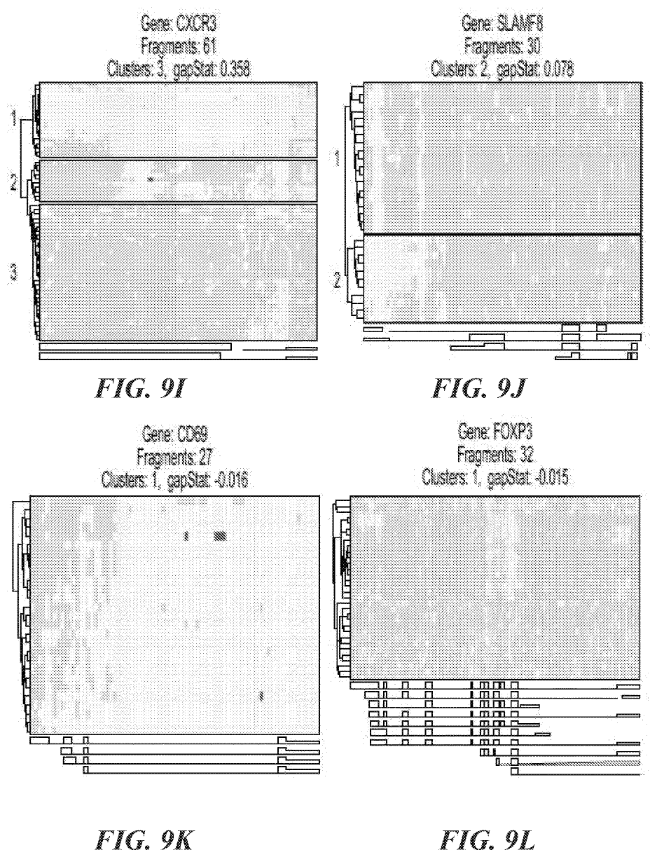

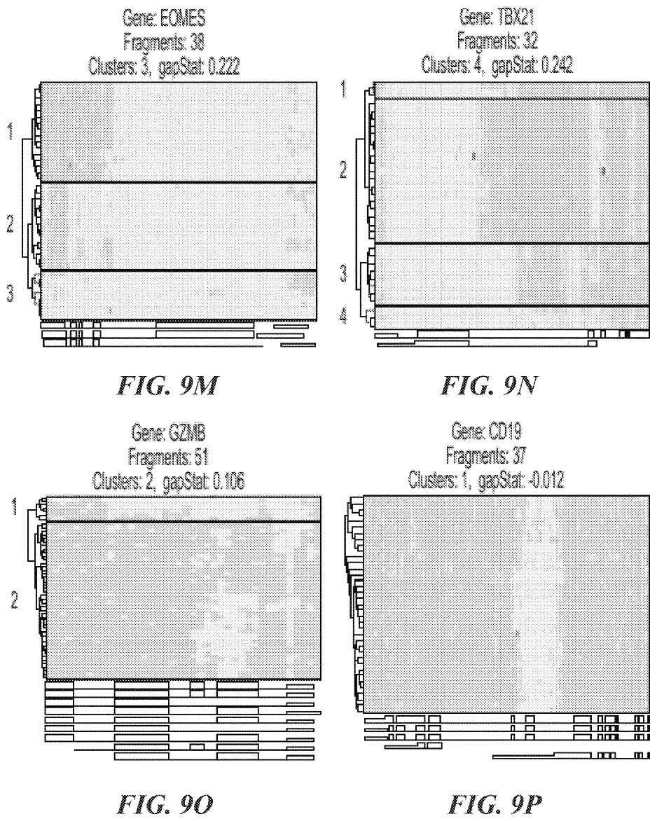

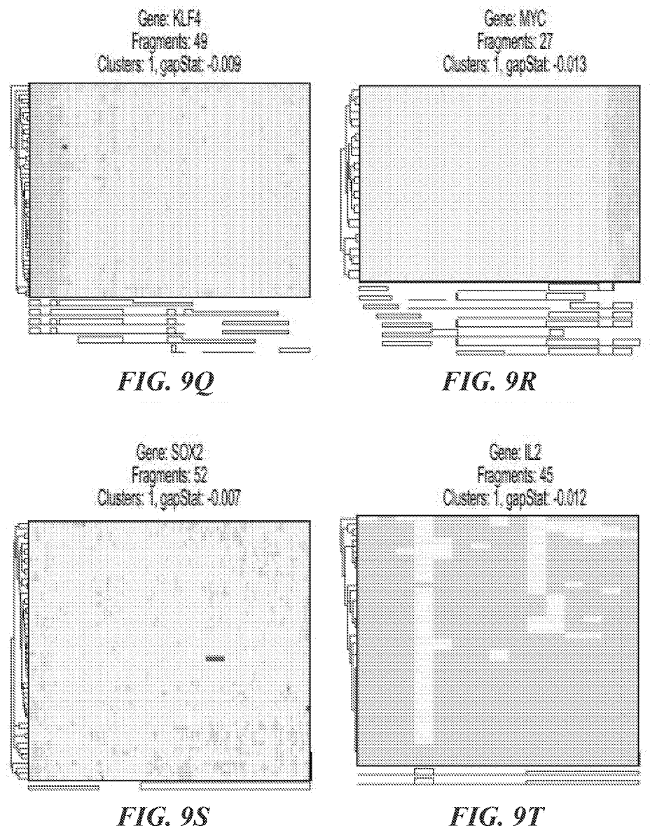

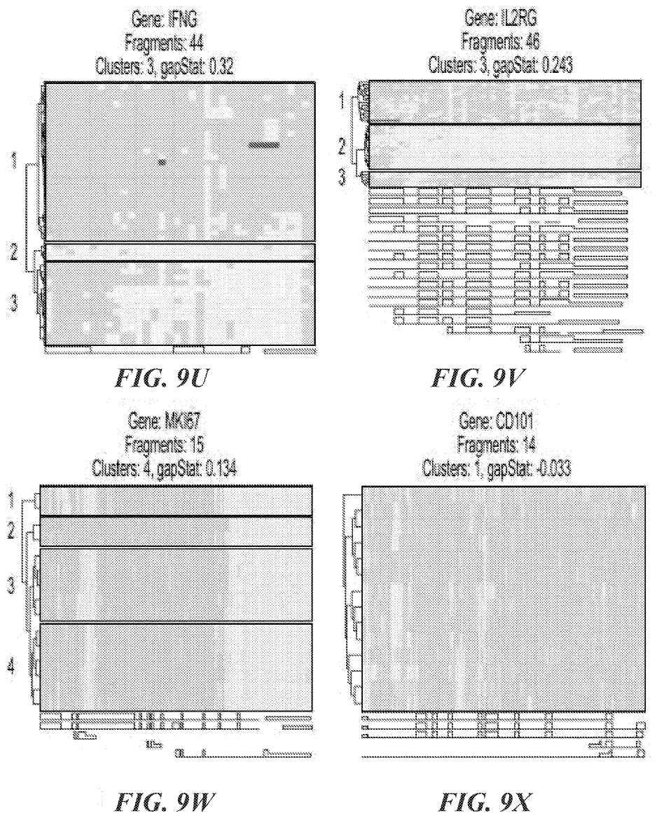

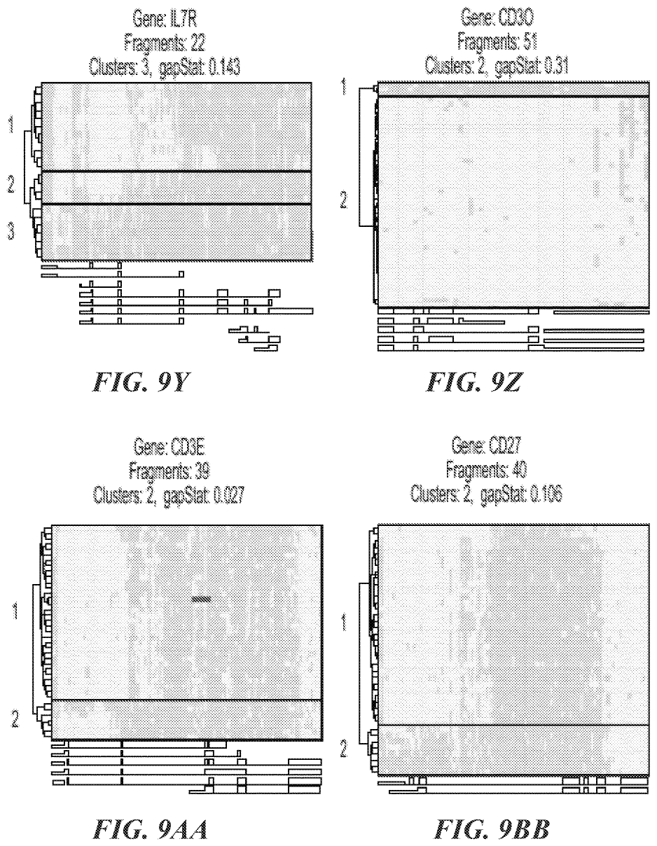

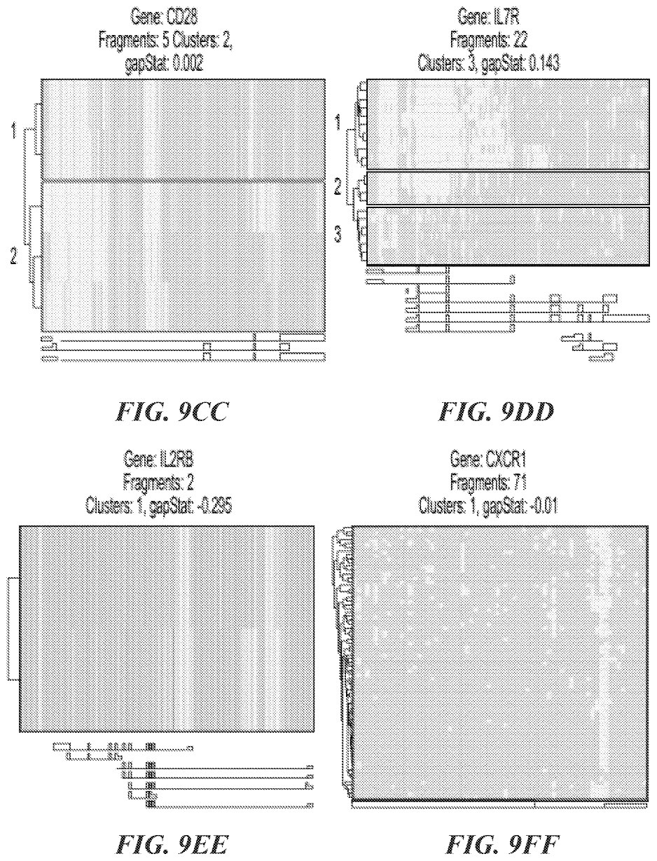

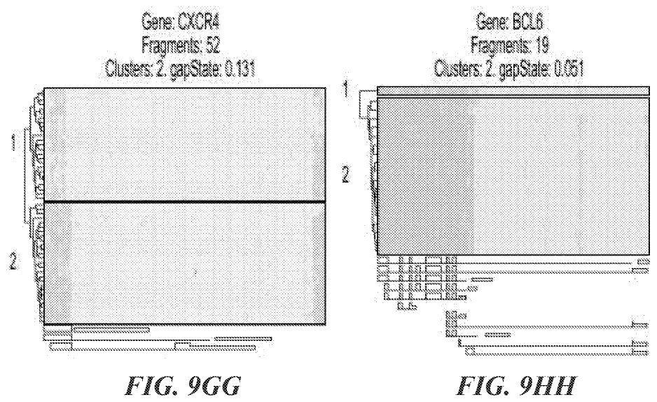

Once the optimal number of clusters was determined, fragments were assigned to the appropriate cluster. Finally, annotations for each CpG based on the UCSC database were added. An example plot for the TCF7 gene is shown in FIG. 8. One of the primary differences between the two clusters appeared to be the methylation of a large intron (shortest gray bar height in FIG. 8). Aside from plot for TCF7, various heatmuaps of T-cell related genes was also generated to show optimal number of clusters based on the Gap Statistic (FIGS. 9A-14Z, FIGS. 9AA-9HH).

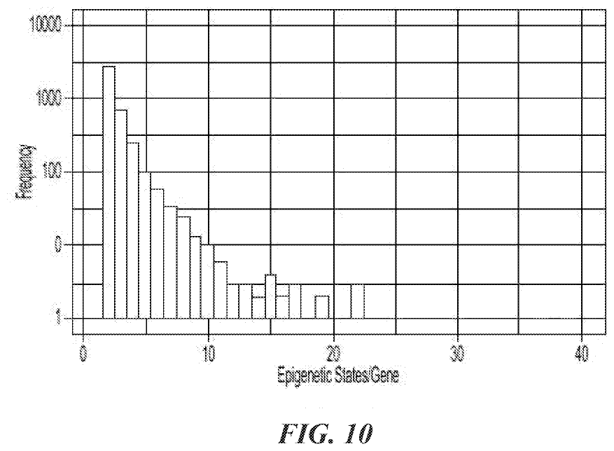

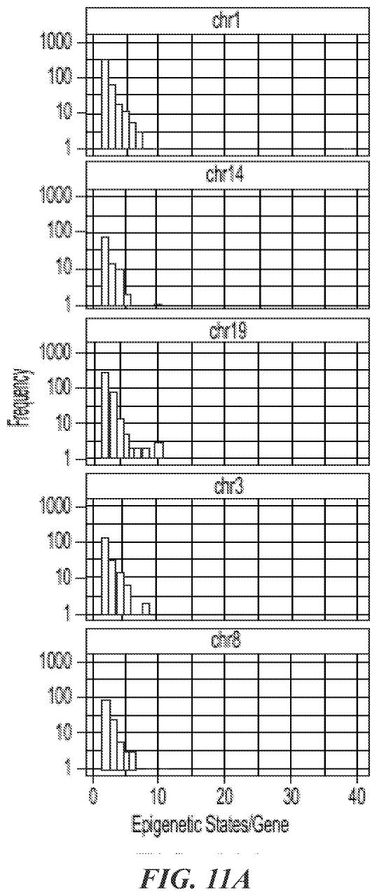

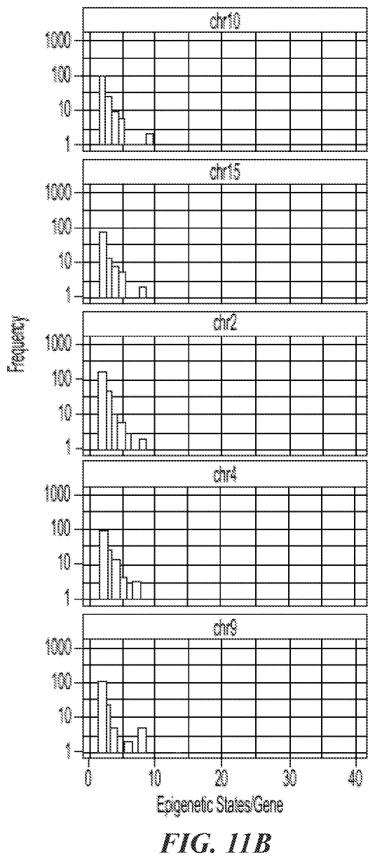

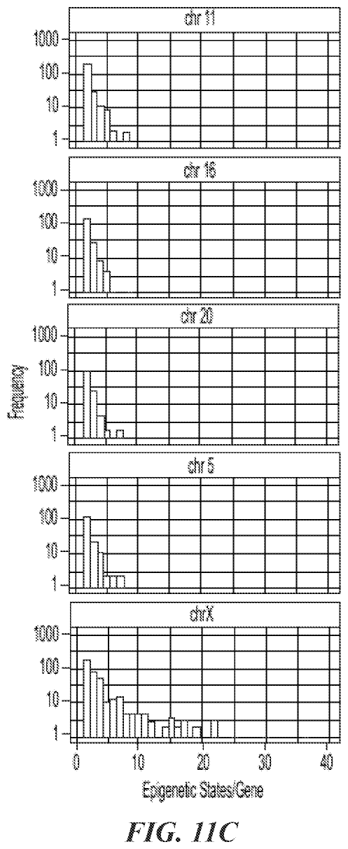

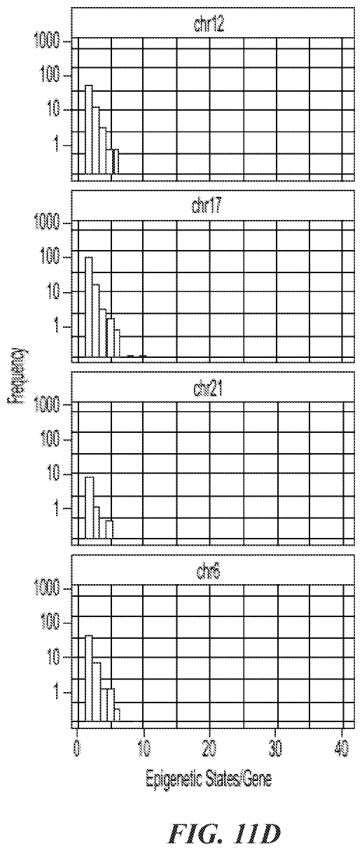

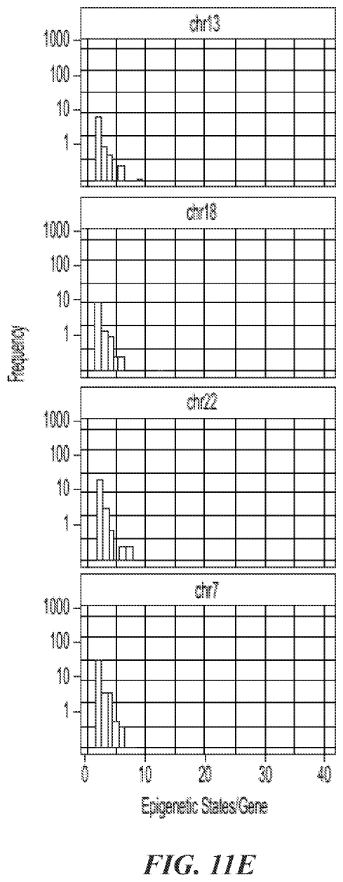

As shown in FIG. 10, distribution of optimal number of clusters based on the Gap Statistic across >14,000 Hg38 genes (y axis is log scale) was generated. While a minority of the overall distribution, genes exhibiting high numbers of clusters (>5-10) can likely be over-clustered (i.e., clusters that do not correspond to true epigenetic states). Similarly, the optimal number of clusters was identified per chromosome. As shown in FIG. 11A-E, the majority of the genes with a large number of epigenetic states appeared to come from X chromosome.

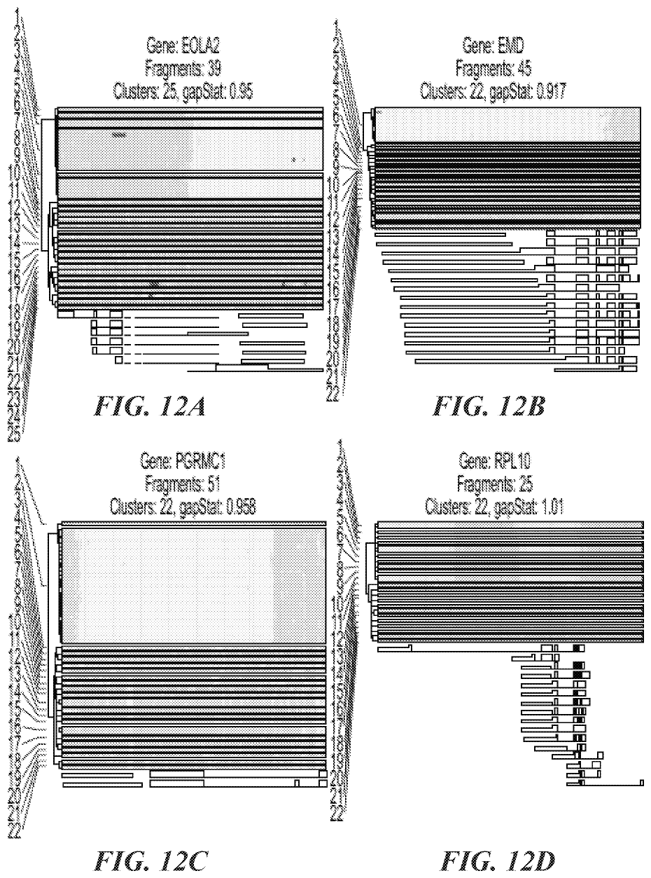

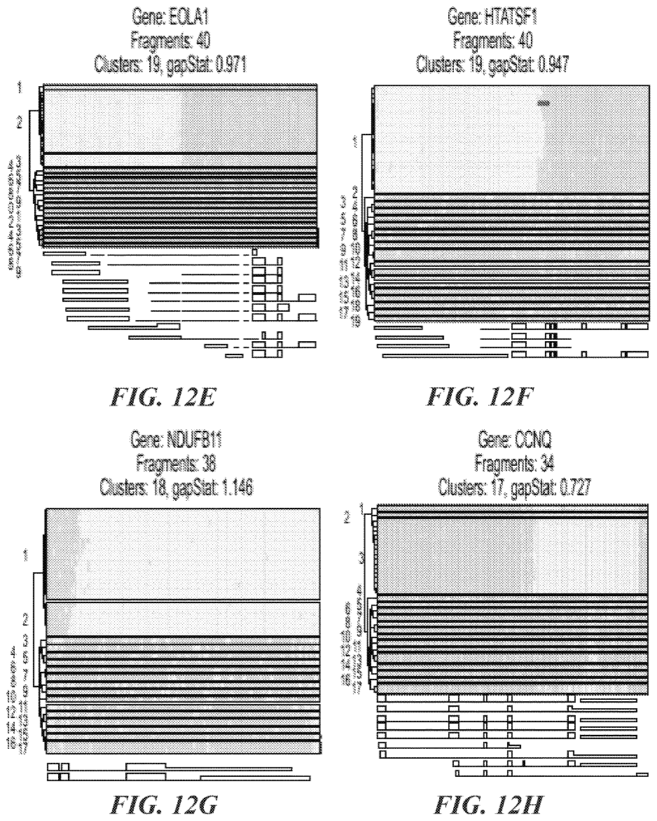

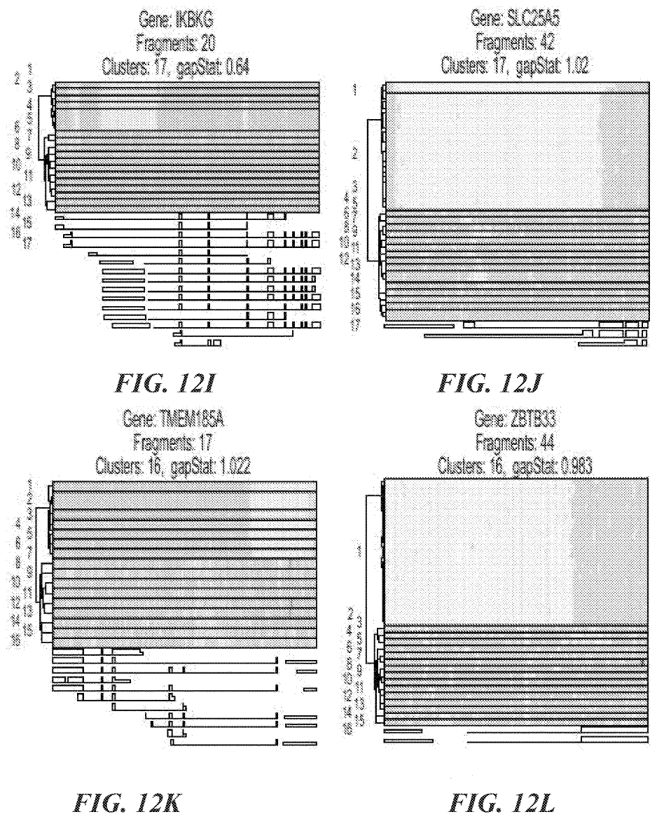

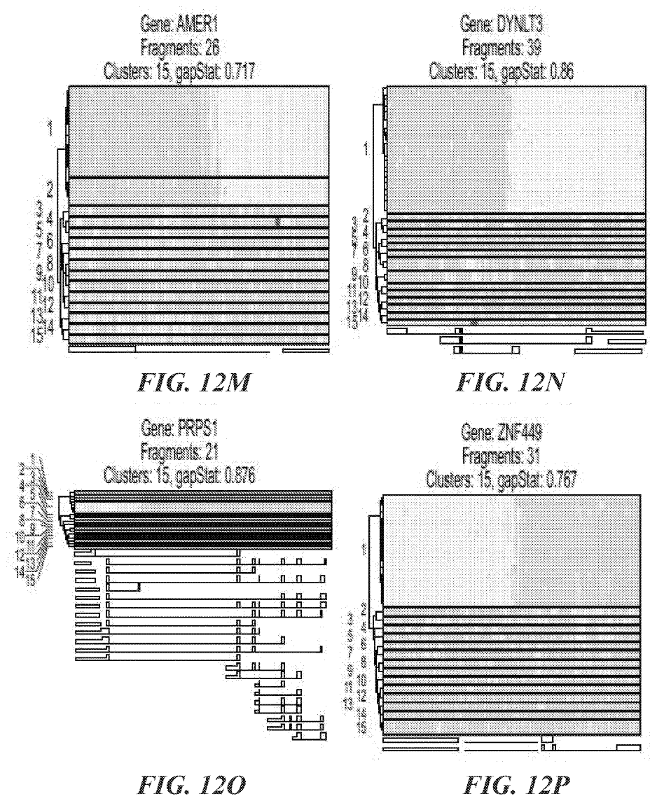

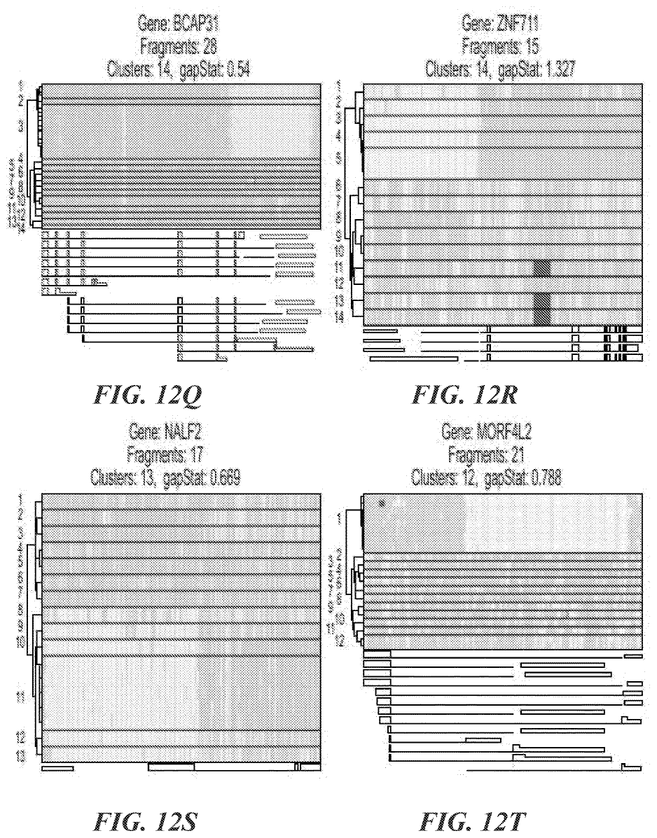

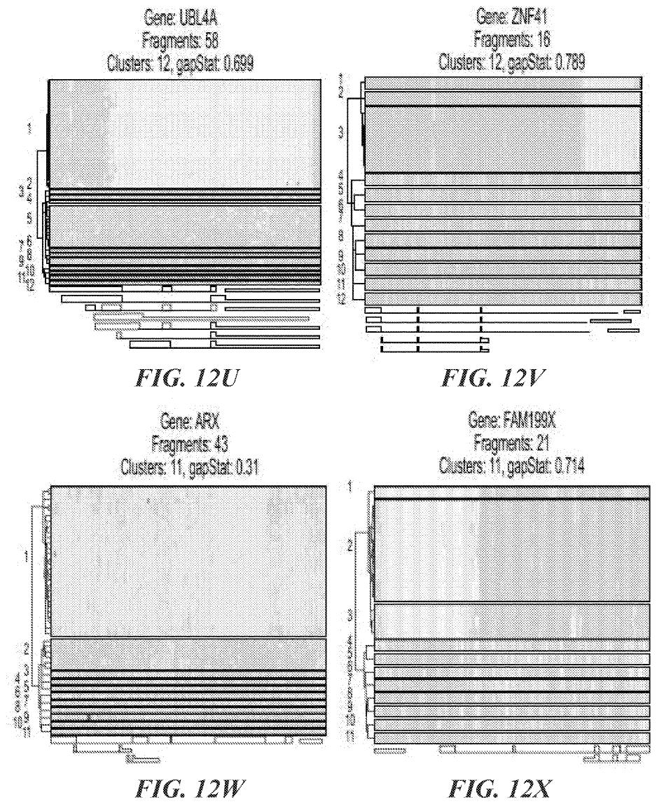

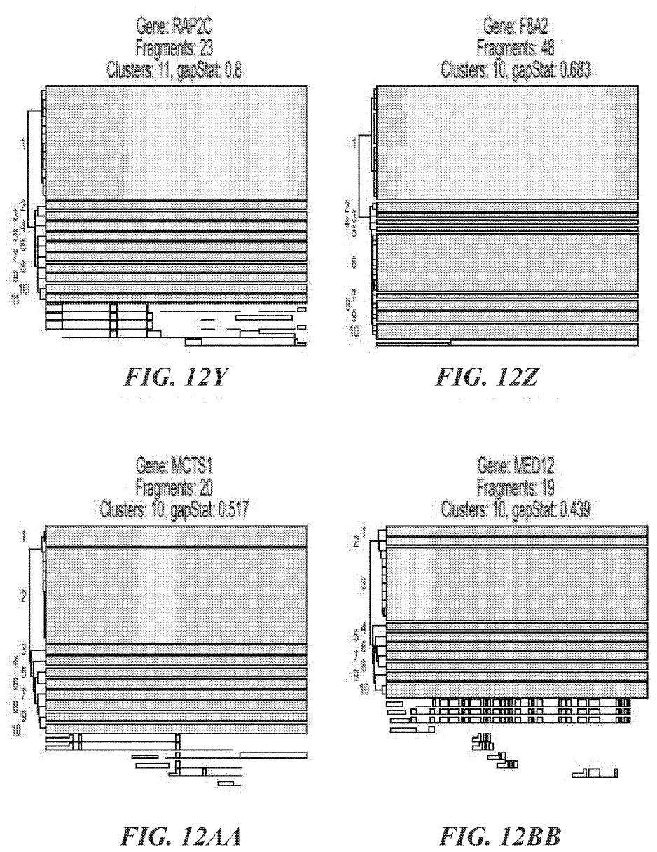

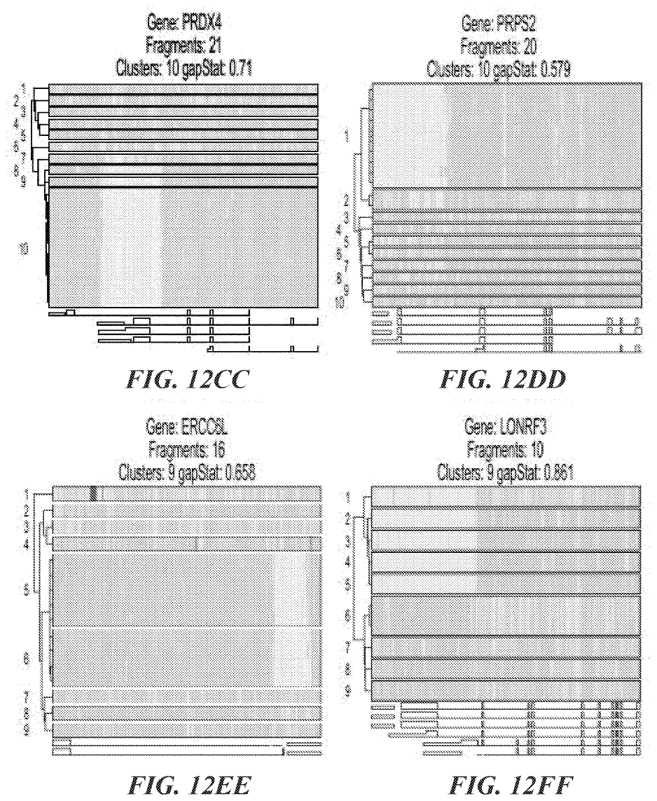

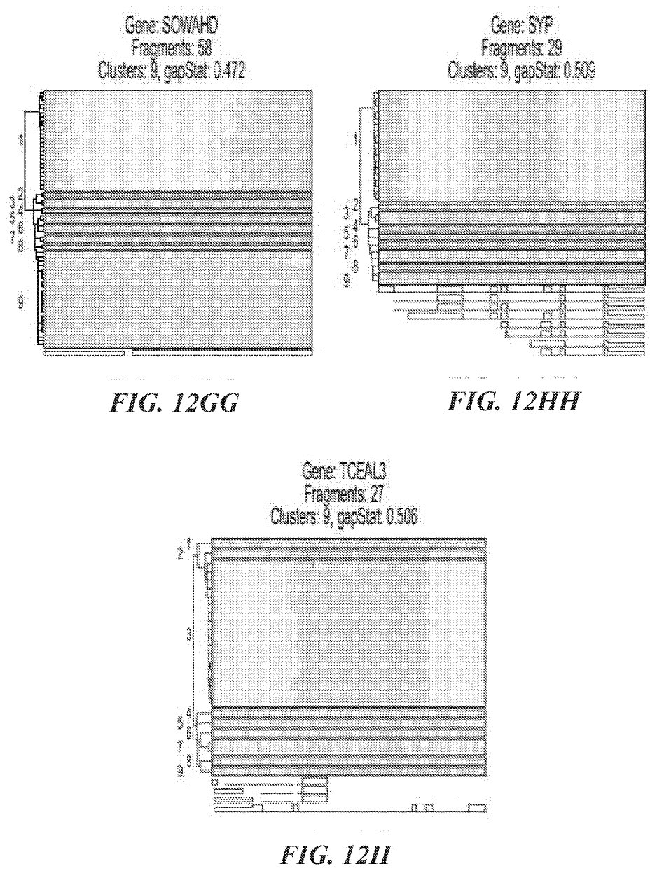

Looking more closely at the chromosome (FIGS. 12A-12Z, FIGS. 12AA-121I), a pattern was observed, where about 50% of the fragments were heavily methylated and clustered separately, while the remaining 50% were clustered with the large coherent regions of methylated/unmethylated CpGs. This may be indicative of X-activation, as all the donors in this data set were female.

Collectively, the implementation of unsupervised clustering analysis method enabled the definition of epigenetic states at the gene level. The unsupervised clustering analysis method can be used for multi-gene (e.g., whole genome) state profiling by linking the states defined for one gene to those arising from a different gene. This may be accomplished through the use of fragments that span multiple genes (thereby enabling one to understand inter-genie correlations of epigenetic states). Alternatively, the inter-genic state relationships using other data modalities such as single cell methylation profiling and/or gene expression may be mapped. Ensuring that the resultant clusters represent true epigenetic states can involve optimization methods, such as tightening the gap statistic selection criteria (increasing the number of SE(k+1)'s that Gap(k+1) must be from Gap(k)), placing an upper limit on the number of allowed epigenetic states per gene (currently it is capped by the number of available fragments), denoising techniques to account for technical/biological noise, and incorporating various heuristics (e.g. weighting CpGs in promoter regions more heavily than introns in distance calculations, developing heuristics for accommodating known biological phenomenon such as X-inactivation).

### Example 2: Assessing the Relative Importance of CpGs to a Given Classification

Long read ONT data from CD8+ T-cells were used to assess the relative importance of CpGs to a given classification (e.g., cluster, experimental condition), which can aid in differential analysis to identify favorable epigenetic editing target sites. Using the ONT data from CD8+ T-cells, the region of interest was selected and subjected to clustering. These clusters then defined the classification. Next, information gain for each CpG in a gene was calculated. Information gain measures the gain in information (reduction in entropy) when partitioning a dataset on a given attribute (e.g., CpG methylation value). Information gain is commonly used in decision tree creation where it is used in a recursive fashion to select the order of attributes to partition on to maximize classification accuracy. Information gain was calculated with the following; equation:
(i) Information Gain=Entropy(T)−Entropy(T|a), where T is a random variable (e.g., epigenetic state) and a is an attribute (e.g., a specific CpG methylation status). Entropy(T|a) can be interpreted as the Expected value of the resulting entropy when the dataset is partitioned on attribute, a. Thus, given knowledge of the methylation of a CpG, how much information is gained regarding the underlying random variable (e.g., epigenetic state) can be calculated.
(ii) Entropy=−p*log 2(p)−(1−p)*log 2(1−p), where p is the probability of event in question (e.g., whether a given CpG is methylated or not).

After the entropy of all clusters (i.e., all fragments) was first calculated, the weighted average of the entropy of each individual cluster of fragments was subtracted. The difference was the information gain.

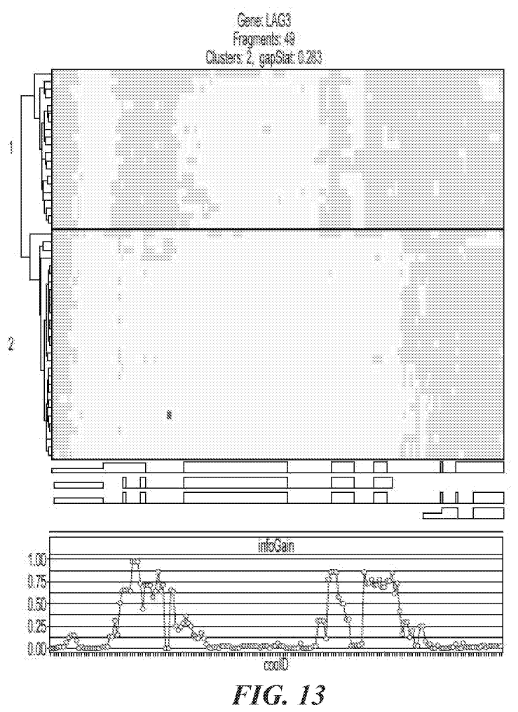

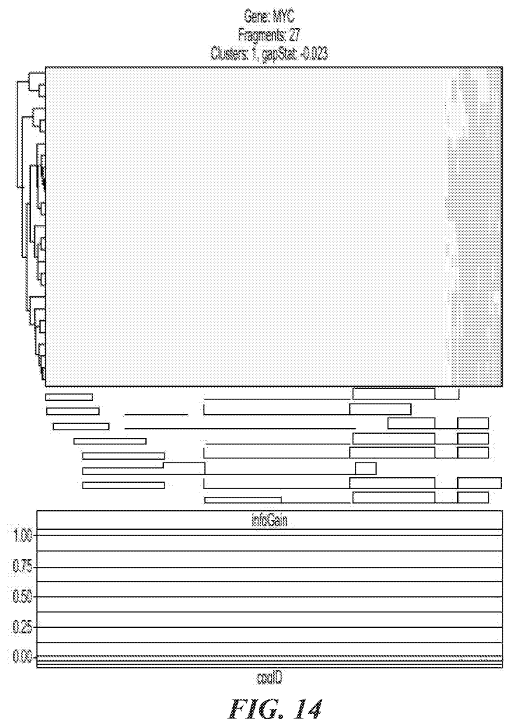

Information gain of various genes provided a method to quantitate the relative importance of a CpG methylation status on the underlying state classification. FIG. 13 shows an example of the calculated information gain for the LAG3 gene. As shown in FIG. 13, regions with high information gain also had clear differences in methylation states between the two clusters. Higher values of information gain indicated those CpGs were more important in defining the clusters. In FIG. 14, the MYC gene had only one cluster; thus, the information gain is zero.

The knowledge of the relative importance of various CpG to some classification (e.g., epigenetic state, experimental condition) afforded the ability to determine which CpG/genomic locations were most important in classification. This information can be used in applications including decision-tree based classification, targeted assays (e.g., use of panels vs. whole genome sequencing), or fundamental understanding of underlying biological processes (e.g., correlating regions of high information gain to differential expression of genes).

### Example 3: Library Preparation for Long Read Whole Methylome Sequencing with Average Reads of ˜30 Kb in Length

This example shows a method of preparing a sequencing library for long read whole methylome sequencing with average N50 reads of ˜30 Kb in length using the Oxford Nanopore Technologies Sequencing platform.

### DNA Shearing, End-Repair, and Purification

In this example, purified genomic DNA was first sheared using a 26 gauge blunt end needle (ThermoFisher UK Ltd HCA-413-030Y CC Syringe Replacement Parts 26 g, 51 mm) attached onto a 1 ml luer-loc syringe. The needle and syringe were used to draw up a sample of cell free DNA (3 ug of DNA in a volume of 50 μL of 10 mM Tris HCl pH8.0, 0.1 mM EDTA) in a 15 μL LoBind sample tube. Once all the liquid from the bottom of the tube was drawn into the needle, the sample was expelled back into the tube. The operation of drawing and expelling the sample with the syringe and needle was repeated 4-5 times to shear the DNA.

End repair was performed on the sheared DNA by preparing the following mix in a 0.2 mL thin-walled PCR tube: 47 μl of the sheared DNA, 1 μL of DNA Control Sample (optional), 3.5 μL NEBNext FFPE DNA Repair Buffer, 2 μL NEBNext FFPE DNA Repair Mix (NEB, M6630), 3.5 μL Ultra 11 End-prep Reaction buffer, and 3 μL Ultra 11 End-prep Enzyme mix (NEB, E7546) and incubating in a thermocycler with the following thermal program: 1) 20° C. for 5 min., 2) 65° C. for 5 min.

The end-repaired DNA was then purified using AXP beads (included in the Ligation Sequencing Kit V14, Oxford Nanopore catalog #SQK-LSK114). A volume of 60 μL of resuspended AXP beads were added to the end-prep reaction and mixed by flicking the tube. The mixture was incubated for 5 min. at room temperature. The sample was spun down and pelleted on a magnet for 10 minutes until the supernatant was clear and colorless. The supernatant was removed and the beads were washed with 200 μl of freshly prepared 70% ethanol while the tube was kept on the magnet to not disturb the pellet. The 70% ethanol was removed using a pipette and discarded. The beads were washed a second time with 200 μl of freshly prepared 70% ethanol and following removal of the ethanol, were resuspended in 61 μL of nuclease-free water and incubated for 2 min. at RT. The tube was placed back in the magnet for 1 min., following which the supernatant was transferred into a clean 1.5 mL low binding tube, and 1 μL was quantified in Qubit.

### Adapter Ligation and Clean Up

The following mixture was prepared for adapter ligation, by adding in the following order into a 1.5 mL Eppendorf DNA LoBind tube: 60 μL of the purified end-repaired DNA, 25 μL of Ligation Buffer (LNB) from the Ligation Sequencing Kit, 10 μL of NEBNext Quick T4 DNA Ligase, and 5 μL of Ligation Adapter (LA). The reaction mixture was incubated for 10 minutes at room temperature. To purify the library, a volume of 40 μl of AXP beads provided in the ligation kit, were added to the reaction and incubated for 10 minutes at room temperature, mixing the sample gently every 30 seconds. The sample was spun down and pelleted on a magnet. While the tube was kept on the magnet, the supernatant was removed. The beads were washed by resuspending in 250 μl Long Fragment Buffer (LFB), spun down, and pelleted for at least 5 minutes on a magnetic rack before removing the supernatant. The beads were washed a second time with 250 μl Long Fragment Buffer (LFB), spun down, and pelleted on the magnet before removing any residual supernatant. The beads were allowed to dry for ˜30 seconds, taken off the magnetic rack, resuspended in 25 μl Elution Buffer (EB), and incubated for 10 minutes at 37° C., The beads were then pelleted on a magnet for 10 minutes until the eluate was clear and colourless before transferring 25 μl of eluate containing the DNA library into a clean 1.5 ml Eppendorf DNA LoBind tube. Then, 1 μl of eluted sample was quantified using a Qubit fluorometer, and the library was sequenced in three split into three libraries of 300 ng (10-20 fmol) in 32 μl using Elution Buffer (EB). Each of the three aliquots of the library was loaded when 25% of the sequencing pores lost their sequencing capacity, by mixing 300 ng of library in 32 μl of Elution Buffer (EB). This procedure yields ˜90 Gb, and ˜30× coverage across the genome.

### Example 4: Preparing Epigenetic Maps of Different T-Cell Differentiation States Using Long-Read Sequencing

In this example, long-read sequencing was used to prepare high resolution epigenetic maps of four different populations of CD8-+ T-cells in different cellular differentiation states, enabling identification of target genomic regions within a gene or a regulatory region for epigenetic editing for modifying a differentiation state of a CD8+ T cell. Epigenetic maps of the four differentiation states were generated from long-read methylation sequencing data using the method of unsupervised clustering of epigenetic states, described in Example 1, yielding information on the methylation states at the gene level across gene loci for the whole genome. The differential between the epigenetic maps of the four differentiation states was used to identify target genomic regions within a gene or a regulatory region for epigenetic editing for modifying a differentiation state of a CD8+ T cell.

To profile CD8+ T-cells in different cellular differentiation states, CD8+ T-cells from a donor were first sorted by fluorescence activated cell sorting (FACS) into the following populations: Naïve CD8+ T-cells, 2) central memory CD8+ T-cells, 3) effector memory CD8+ T-cells, and 4) effector CD8+ T-cells and sequenced by whole methylome sequencing across the whole genome using long read ONT sequencing. Epigenetic maps for the whole genome (20,0000+ genes) were prepared showing methylation sites for each population. The epigenetic maps were used to assess the differences in methylation states across each gene locus, including CpG sites, for different CD8+ T-cell differentiation states.

Sorting CD8+ T-Cell Differentiation Subsets from Donor T-Cells and Whole Methylome Sequencing

### Cell Thawing and Incubation

T cells from a donor were thawed and incubated overnight to allow for re-expression of cell surface markers including CD62L in preparation for staining and sorting. Vials of PBMCs from donor TIS006, CEL021, Aliquot CHS-0001504791 were taken from a liquid nitrogen stock and thawed in a 37° C. water bath for 2-3 minutes or until only small chunks of frozen contents can be visualized. A 1 mL volume of pre-warmed PBMC thaw medium (10% Heat Inactivated Fetal Serum, 1 μg/mL DNAseI in 1× Phosphate Buffered Saline (PBS)) was slowly added into each PBMC cryopreserve vial in a drop-wise manner. The cells were mixed by gentle pipetting and then diluted in pre-wanned PBMC cell thaw medium, such that the final volume of PBMC cell thaw medium to cryopreserved cell stock is at 10:1 (v:v) ratio. Multiple PBMCl vials from the same donor can be thawed and pooled by scaling the volume of the PBMC thaw proportionally. The cells were centrifuged at 600×g for 5 minutes at room temperature. The cells were resuspended in culture media (RMPI 1640+10% FBS+1× Glutamax) at a concentration of 10,000,000 cells/mL. The cells were incubated overnight to allow for re-expression of CD62L and other cell surface markers. The following day, CD8+ T cells were isolated from the PBMCs utilizing the StemCell Human CD8+ T cell Isolation kit.

### Cell Staining

In total, about 100 million cells were stained in preparation for sorting. The following antibodies were used for staining: APC anti-human CD45RO and an anti-human CD62L antibody.

Following enrichment of CD8+ T cells, the T cells were spun at 600×g for 5 minutes and resuspended in 2 mL of FACS buffer (Mg²⁺/Ca²⁺-free 1×PBS+2% HI FBS). Aliquots of 10 μL of cells were put aside for the following: blank, 7AAD-only, CD45RO-only, and CD62L-only for single staining (sorter compensation). The volume of each was brought up to 200 μL with FACS buffer. The remaining 1960 μL of cells were stained for sorting. For staining, 20 μl of each antibody stock was used to stain cells, according to the experimental condition. The cells were incubated for 30 min. at 4 C. Following incubation, the cells were washed three times by adding 10 mL of FACS buffer and centrifuged at 300×g for 5 min. at 22° C. and removing the supernatant. An aliquot of 10 μL of double-stained cells were put aside to incubate for 5 min. at 70° C. as a positive control of dead cells (7AAD+).

### Sorting

Before sorting the cells into tubes, the respective percentages of the naïve CD8+ T-cell population (CD62L+/CD45RO−), the central memory CD8+ T-cell population (CD62L+/CD45RO+), the effector memory CD8+ T-cell population (CD62L−/CD45RO+), and effector CD8+ T-cell population (CD62L−/CD45RO−) were verified using the CD62L and CD45RO markers.

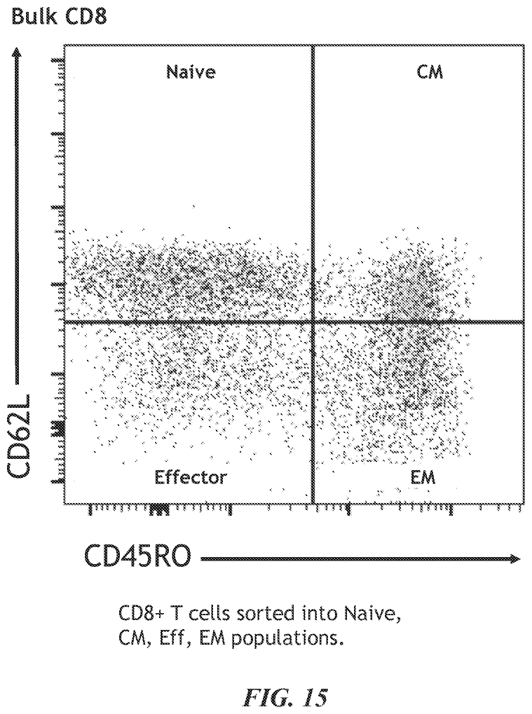

The T-cells were then sorted into the populations: 1) Naïve CD8+ T-cells, 2) central memory CD8+ T-cells, 3) effector memory CD8+ T-cells, and 4) effector CD8+ T-cells, as shown in FIG. 15, and index sorted into 5 mL FACS tubes or 15 mL conical tubes. The sorted cells can be stored at −80° C. until ready to use for library preparation. The genomic DNA from the sorted cells were then extracted. Sequencing libraries were prepared from the genomic DNA and sequenced using ONT sequencing.

### Preparation of Epigenetic Maps and Identification of Target Epigenetic Editing Regions

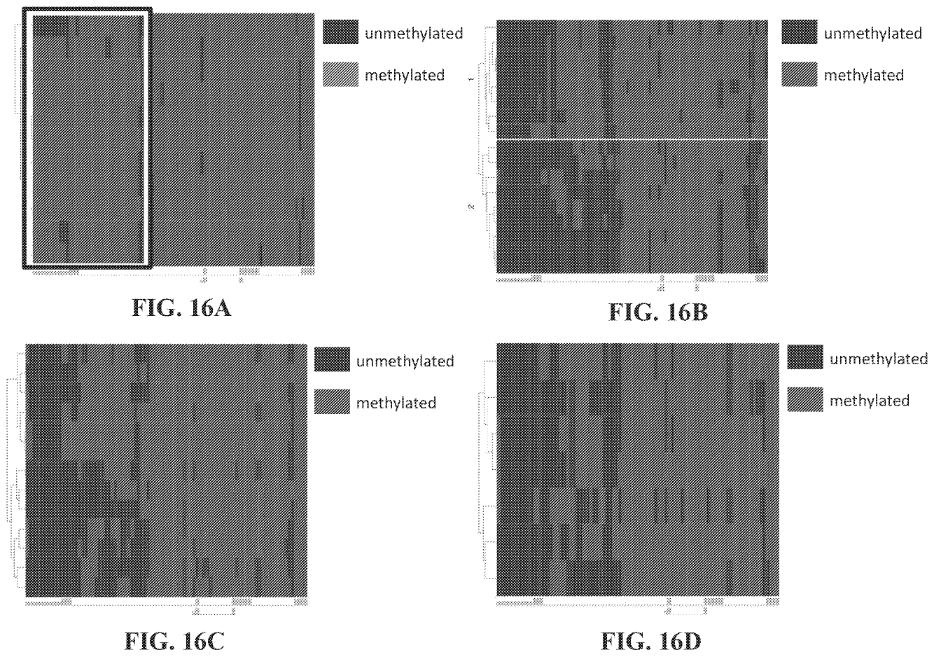

The sequencing results were used to prepare epigenetic maps that are specific to each CD+ T-cell differentiation subset for 20,000-+ genes in the genome using a method of unsupervised clustering of epigenetic states, as described in Example 1. Each epigenetic map shows methylation states across each gene locus collected from the methylome sequencing results of a particular CD8+ T-cell subset. FIGS. 16A-16D show an example of epigenetic maps of the GZMK gene prepared from the sequencing results for the naïve CD8+ T-cells (FIG. 16A), the central memory (CM) CD8+ T-cells (FIG. 16B), the effector CD8+ T-cells (FIG. 16C), and the effector memory (EM) CD8+ T-cells (FIG. 16D). The dark gray bands represent an unmethylated state, while the light gray bands represent a methylated state. The x-axis in each map represents the chromosome position across the GZMK gene region. The y-axis in each map represents an individual sequencing read from a single cell. The blocks below each epigenetic map represent regions representing promoters, introns, and exons.

As FIGS. 16A-16D show, the GZMK gene is overall more highly methylated in naïve CD8+ T cells as compared to the CM CD8+ T-cells, EM CD8+ T-cells, and effector CD8+ T-cells. Comparison of the epigenetic maps in FIGS. 16A-16D revealed a region at the 5′ end of the gene, indicated by the boxed region in FIG. 16A, that showed substantially higher levels of methylation in naïve CD8+ T cells compared to the CM CD8+ T-cells, EM CD8+ T-cells, and effector CD8+ T-cells. Based on this differential between the epigenetic maps, this region was identified as a target region for epigenetic editing. It is predicted that targeting this region for methylation in a CM CD8+ T-cell, an EM CD8+ TF-cell, or an effector CD8+ T-cell may produce a modified CD8+ T-cell that is closer in phenotype/function to a naïve CD8+ T-cell.

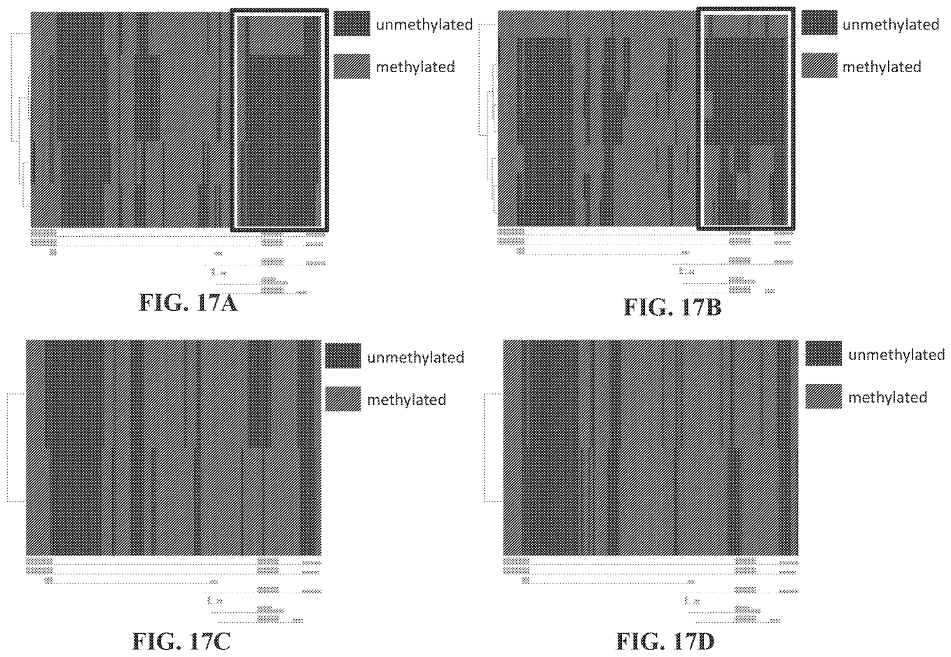

FIGS. 17A-17D show an example of epigenetic maps prepared from the sequencing reads for the SELL gene. The results show that the SELL gene had lower levels of methylation in naïve CD8+ T cells and CM CD8+ T-cells as compared to EM CD8+ T-cells and effector CD8+ T-cells. Comparison of the epigenetic maps in FIGS. 17A-17D revealed a region at the 3′ end of the gene, indicated by the boxed region in FIG. 17A and FIG. 17B, that showed substantially lower levels of methylation in naïve CD8+ T cells and CM CD8+ T-cells compared to effector CD8+ T-cells and EM CD8+ T-cells. Based on this differential between the epigenetic maps, this region was identified as a target region for epigenetic editing. It is predicted that targeting this region for demethylation in an EM CD8+ T-cell or an effector CD8+ T-cell may produce a modified CD8+ T-cell that is closer in phenotype/function to a naïve CD8+ T-cell or a CM CD8+ T-cell.

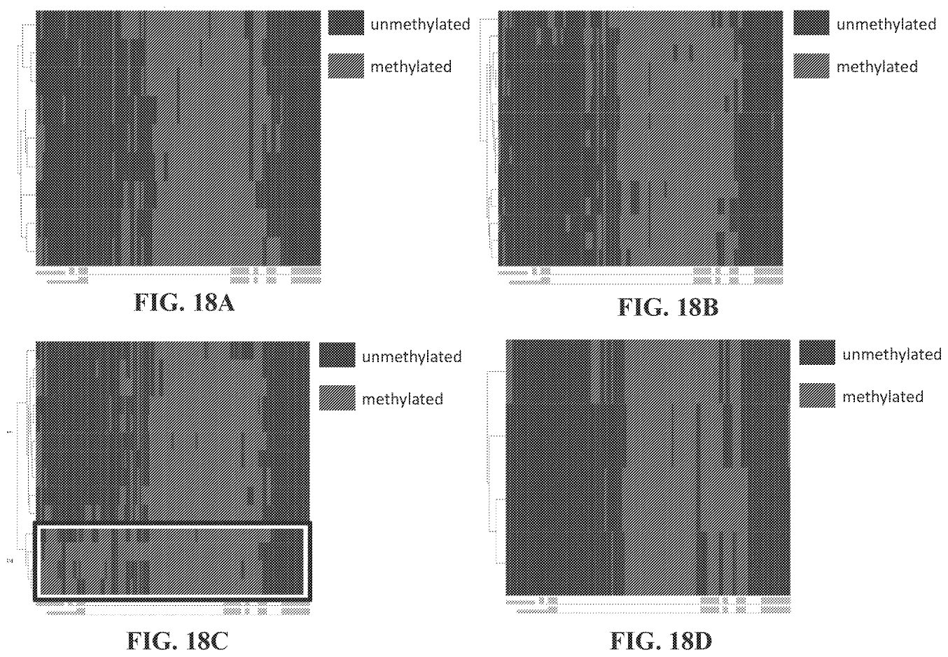

FIGS. 18A-18D show an example of epigenetic maps prepared from the sequencing reads for the CD27 gene. The results show that the CD27 gene had higher levels of methylation in effector CD8+ T cells as compared to naïve CD8+ T cells, CM CD8+ T-cells, and EM CD8+ T-cells. Comparison of the epigenetic maps in FIGS. 18A-18D revealed a region at the 5′ end of the gene, indicated by the boxed region in FIG. 18C, that showed substantially higher levels of methylation in effector CD8+ T-cells compared to naïve CD8+ T cells, CM CD8+, and EM CD8+ T-cells. Based on this differential between the epigenetic maps, this region was identified as a target region for epigenetic editing. It is predicted that targeting this region for demethylation in an effector CD8+ T-cell may produce a modified CD8+ T-cell that is closer in phenotype/function to a naïve CD8+ T-cell, a CM CD8+ T-cell, or an EM CD8+ T-cell.

Beyond the genes shown in this example, epigenetic maps were also prepared for the four CD8+ T cell subsets for each gene in the human genome. Differential analysis can be conducted to identify target regions in different regions in these genes for epigenetic editing with the goal of modifying a CD8+ T-cell in one differentiation state to produce a CD8+ T-cell in another differentiation state.

### Example 5: Preparing Epigenetic Maps of Different Cell/Tissue Types to Identify Target Sites for Selective Editing of Specific Cells/Tissues

When introducing epigenetic edits to cells, such as for the purpose of modifying a cellular state, it may be desirable to control the effects of epigenetic editing to specific target cell types and minimize modifications to off-target cell types/tissues. This example shows a method of using high resolution epigenetic maps to enable identification of favorable epigenetic editing target sites in a target liver hepatocyte that would introduce minimal modifications to off-target cells of another cell/tissue type.

In this example, high resolution epigenetic maps of cells of different cell types were compared to inform the selection of epigenetic editing target sites in target liver hepatocytes that would minimize the level/risk of undesired epigenetic editing in other off-target cell types and tissues.

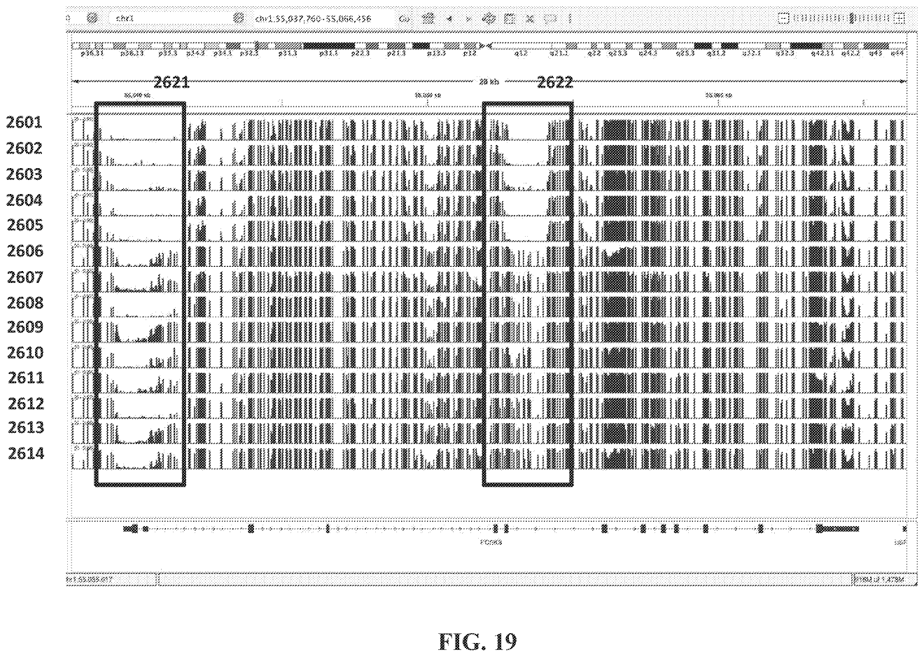

Epigenetic maps were constructed from a public data set of whole genome methylation data of different cell types. As shown in FIG. 19, the epigenetic maps depict methylation of the genomic sites within the PCSK9 gene and the promoter region of the PCSK9 gene. From top to bottom, 2601, 2602, 2603, 2604, and 2605 in FIG. 19 are five epigenetic maps of liver hepatocytes, 2606 is an epigenetic map of liver macrophages, 2607 is an epigenetic map of liver endothelium cells, 2608 is an epigenetic map of gastric body epithelium cells, 2609 is an epigenetic map of pancreas alpha cells, 2610 is an epigenetic map of pancreas ductal cells, 2611 is an epigenetic map of pancreas beta cells, 2612 is an epigenetic map of pancreas acinar cells, 2613 is an epigenetic map of pancreas delta cells, and 2614 is an epigenetic map of pancreas endothelium cells. Within each epigenetic map, the height of the blue bars represents the degree of methylation, with tall bars representing genomic sites with high methylation levels and short bars or non-existent bars representing genomic sites with low methylation levels or unmethylated genomic sites.

In this example, liver hepatocytes were designated as the target cells and the other cell types were designated as off-target cells. Based on the liver hepatocyte epigenetic maps, two substantially unmethylated regions (that are boxed) were identified as potential target regions for methylation. The first boxed region 2621 comprises the promoter region of the PCSK9 gene. The second boxed region 2622 comprises a region within the PCSK9 gene body. Comparison of the liver hepatocyte epigenetic maps and the epigenetic maps of the other off-target cell types shown in FIG. 19 revealed that the second boxed region within the PCSK9 gene body is substantially unmethylated in liver hepatocytes but substantially methylated in other off-target cell types, suggesting that this region would be a favorable target region for methylation in liver hepatocytes. Since the lack of methylation in this second boxed region is specific to liver hepatocytes, it was predicted that that targeting; this region for methylation would produce the intended modifications to the liver hepatocytes, while minimizing the risk/degree of unintended modifications to the off-target cell types (which are already substantially methylated in this region). On the other hand, comparison of the epigenetic maps in FIG. 19 revealed that the promoter region of the PCSK9 gene is substantially unmethylated in liver hepatocytes and in the other off-target cell types, suggesting that the promoter region of the PCSK9 gene may be a less favorable target region for methylation given the larger risk of considerable modification to off-target cell types. Since the promoter region of the PCSK9 gene is substantially unmethylated across multiple cell types, it was predicted that targeting this region of the genome for methylation would simultaneously methylate this region in the target liver hepatocytes and in the off-target cells unless other measures are put in place to selectively target the liver hepatocytes.

As shown in this example, comparison of epigenetic maps of different cell and tissue types can reveal genomic regions that are specifically methylated or unmethylated in certain cell/tissue types, which may inform selection of target sites for epigenetic editing that would minimize modifications to off-target cells/tissues. By identifying target sites that are in an undesired methylation state in the target cell but are already in the desired methylation state in off-target cells/tissues, one can safely introduce a targeted epigenetic intervention that only modifies the intended target cell and does not affect the off-target cells/tissues. For instance, if a target genomic site is substantially unmethylated in liver hepatocytes but already substantially methylated in off-target cells/tissues, then introducing an methylase fusion protein targeting the target genomic site would modify the liver hepatocytes but minimize modifications to the off-target cells/tissues, which are already methylated in the target genomic site.

This strategy of using differential epigenetic maps of different cell/tissue types can be useful for targeting any cell/tissue type with minimal modifications to another off-target cell/tissue, by revealing methylation patterns that are unique to the target cell/tissue type.

This strategy considerably reduces the search space for favorable target genomic sites for epigenetic editing. For particular applications, the methods described in this example (that identify an editing region would that minimize unintended modifications to off-target cell types) can be combined with the methods that identify an editing region for the purpose of modifying a cellular state. By combining these methods, one can identify a target epigenetic editing site that would both serve in modifying a target cell from an initial cellular state (e.g. a highly differentiated state) to a desired cellular state (e.g., a less differentiated state) and also minimize unintended modifications to off-target cell types.

### Example 6: Overview of Reprogramming Cells with sgRNA and Effector Library

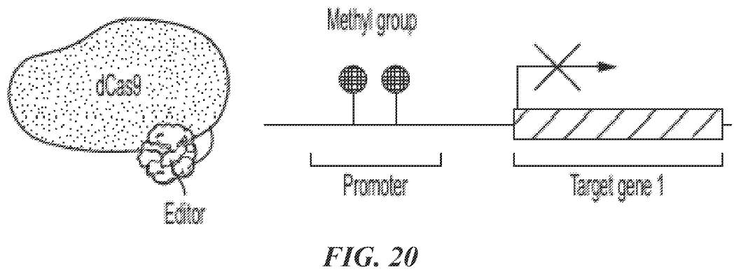

This example provides an overview of a method of introducing targeted epigenetic edits to target genomic sites using CRISPR-based epigenetic editing systems, as described in certain embodiments herein. In some embodiments, CRISPR-based epigenetic editing systems comprise an epigenetic modulator and a guide RNA that targets the epigenetic modulator to a target nucleic acid site, where the epigenetic modulator introduces an epigenetic edit (e.g., methylation or demethylation of the target site). One example of an epigenetic modulator is a dCas9 fused to an effector moiety (e.g., methylase). A guide RNA targeting a specific promoter region of target gene 1 can guide the epigenetic modulator to the target site, where the effector moiety methylates the target site, thereby silencing gene expression of target gene 1, as depicted in FIG. 20. In this example, a target list of one or more CpG targets and associated effector types is provided by data or an artificial intelligence (AI) core. This can include targets sites identified from differential analysis of epigenetic maps identifying favorable epigenetic editing target sites. This can include target sites identified from differential analysis of epigenetic maps of two different cellular states (e.g., two different differentiation states), epigenetic maps of two different cell types, or a combination thereof. In some cases, data is provided to an artificial intelligence (AI) core, which is trained to conduct such differential analyses and identify favorable epigenetic editing target sites.

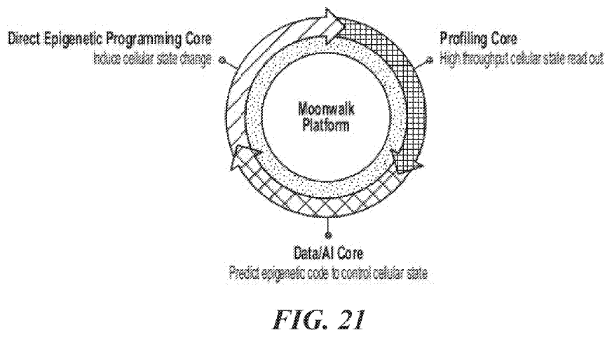

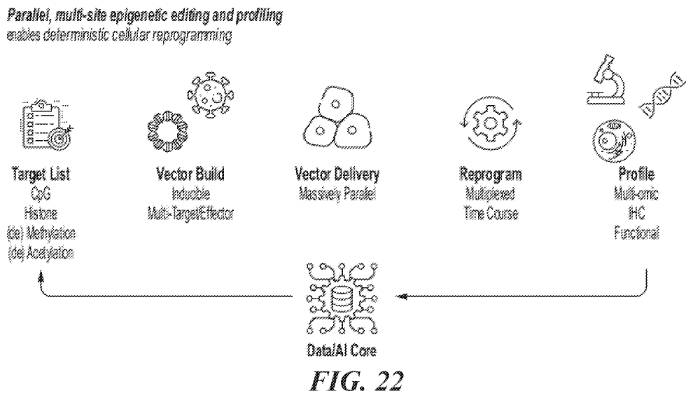

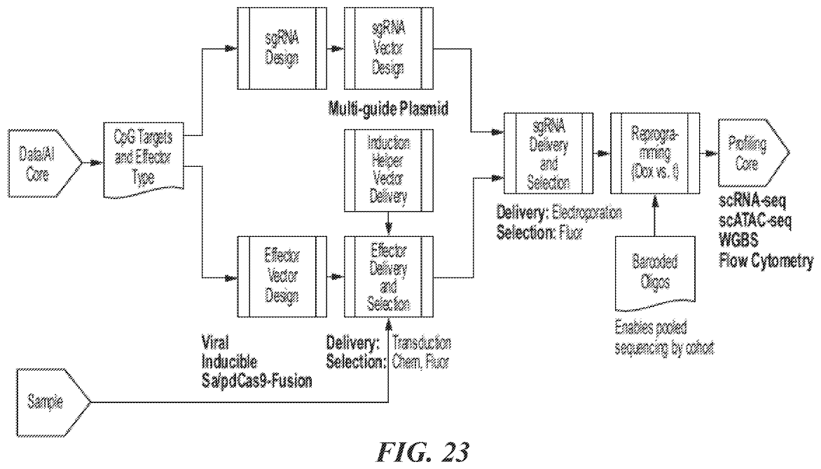

As shown in FIG. 21, FIG. 22, and FIG. 23, data/AI core can determine a list of targets (e.g., CpGs, histones, transcription factors, proteins) that are required to be augmented into to implement a specific reprogramming protocol. This target list is used to generate a guide RNA library specific to each CpG location. One or more guide RNAs are placed on the same transfer plasmid.

In parallel to the guide RNA plasmid library construction, an effector library is designed to deliver the required effector types. Vectors are built to specifically modify the epigenonme (e.g., CpG methylation, histone acetylation). These effectors may be inducible and target multiple epigenetic loci and elicit different effector function (e.g., methylation vs. demethylation) to achieve parallelized modification of the epigenome. In this embodiment, this may be a library of native dCas9 and dCas9 fusion proteins specific to (de)methylation and/or (de)acetylation. The dCas9 variety may be from the aureus or pyogenes lineage. This effector library is loaded into one or more viral vectors (e.g., LVV, AAV), transduced into the sample or cells of interest, and reprogramming is initiated. Optionally, a second class of viral vectors may be transduced into the sample, which enables the dCas9 construct to be expressed in the presence of an induction reagent (e.g., Dox). In the case of an inducible system, the reprogramming may be controlled via exposure to a chemical which allows for time-based control of the reprogramming vectors. Sample cells with the desired edits are sorted from cells, which did not receive the edits via a chemical selection or fluorescence reporter.

With the sample containing the effector library, the sgRNA library is then delivered to the sample via electroporation, nucleofection, or other similar techniques. Sample cells that have received the desired edit are selected via a fluorescent reporter.

Sample cells which now have both the sgRNA and Effector library are reprogrammed via a time-coursed exposure to a cocktail containing the induction reagent. Under exposure to the induction reagent, the effector protein is expressed, combines with the sgRNA library and effects the desired epigenetic edit. Multiple reprogramming protocols may be delivered to separate cohorts of the sample and then combined for sequencing by exposing each cohort, prior to combination, to a barcoded oligo that enables downstream deconvolution via sequencing.

Finally, the effectiveness of the reprogramming protocol is assessed via deep multi-omic profiling. The sample cells with desired epigenetic edits are pooled and profiled via a variety of techniques that may include: scRNA-seq, scATAC-seq, WGBS, Flow Cytometry, and Functional Assay. These data are then fed back into the Data/AI core for future optimization and/or improvements.

### Example 7: Targeted Epigenetic Modification of HTEK293 Using a CRISPR Epigenetic Editing System

This example shows an application of high resolution epigenetic maps generated from long-read methylation sequencing to profile cells that have been modified by a CRISPR epigenetic editing system. In this example, CD151 and CD81 in HK293 cells were modified using a CRISPR epigenetic editing system with guide RNAs targeting specific target sites within CD151 and CD81 for methylation. Successful methylation of the targets by the CRISPR epigenetic editing system was inferred upon downregulation of protein expression, which was evaluated using flow cytometry. Changes in DNA methylation patterns were also analyzed using epigenetic maps generated from long-read methylation sequencing results of the edited cells and control cells (cells that were not treated with the guide RNAs). The results showed that in the edited cells, the target site in the CD151 promoter was successfully methylated by the CRISPR epigenetic editing system.

### Optimization of ExpOFF Epigenetic Editing System

ExpOFF epigenetic editing system (e.g., OFF system) was created and optimized to induce epigenetic silencing via transient transfection methods. The ExpOFF system was composed of ZNF10 KRAB, DNMT3A, and DNMT3L domains fused to a catalytically inactive S. pyogenes dCas9. The ExpOFF system served to silence gene expression through DNA methylation at a target site. In this example, CD151 and CD81 were selected as initial targets. Three sgRNAs were designed to target three target sites in CD151 (including one targeting a promoter region), and three sgRNAs were designed to target three targets in CD81.

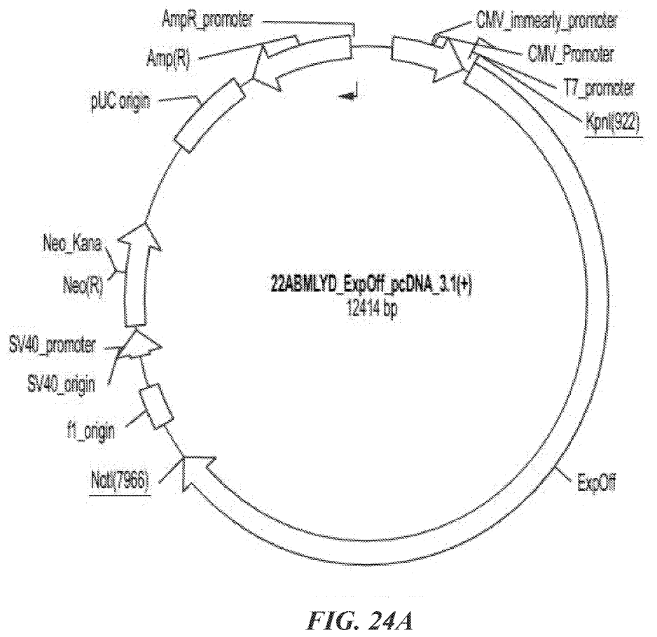

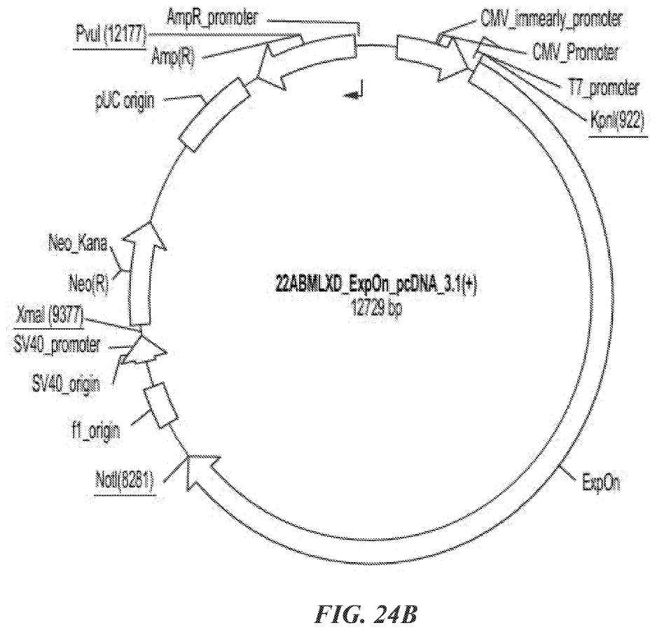

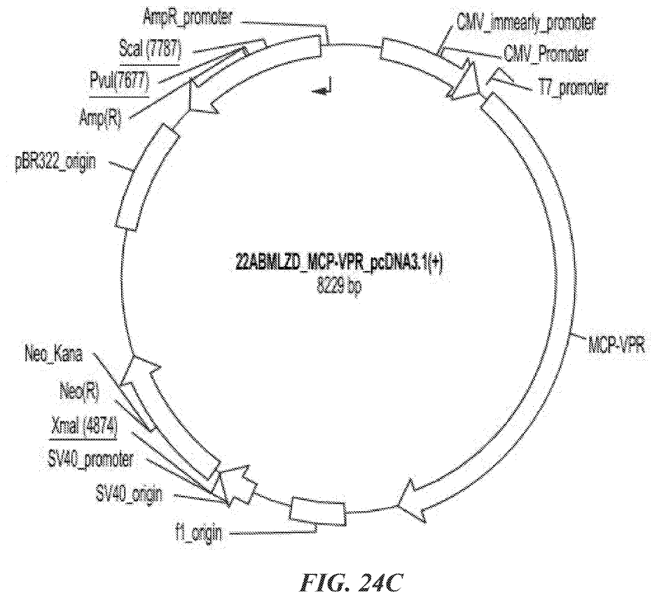

To test the system, Hek293.2sus cells (e.g., ATCC (CRL-1573.3) were cultured and passaged in 293 SFM II media (Gibco CAT #11686029) with 100 units/mL of penicillin/streptomycin (Gibco Cat #15140122) and 4 mM Giutamax (Gibco Cat #35050061). For 3 days post-electroporation, Hek293.2sus cells were cultured in the same media composition as stated above minus the penicillin/streptomycin. Next, cells were collected in a 50 mL Falcon tube then spun down at 300g for 5 minutes and washed with 1×DPBS. The cell pellet was then resuspended in 5 mL of TrypLE 1× (Gibco Cat #12604013) and incubated at RT for 5 minutes. The cell suspension was strained through a cell strainer to remove clumps, followed by cell counting and washing with IX DPBS. The cells were resuspended to a density of 5e7 cells/mL and transfected according to Neon Transfection System 100 uL kit protocol. Electroporation parameters of 1200V/20 ms pulse width/2 pulses were used for all samples. Transfection setup details can be found in Table 5. ExpOFF plasmids (FIG. 24A) were sourced from Thermofisher (GeneART) and sgR-NAs were sourced from Synthego. Sequences of ExpOFF and gRNAs that were used are listed in Table 6. In Table 6, the SEQ ID NO: 15 sequence corresponds to the structural sgRNA component that interacts with the Cas system. The remainder of the sequence is the portion of the sgRNA targeting the gene location of interest. For this experiment, CD151 and CD81 were chosen as initial targets as they are not essential to cell proliferation or survival. In addition, they are highly expressed in HEk293 cell line and are surface markers that can be easily detected in a non-destructive manner.

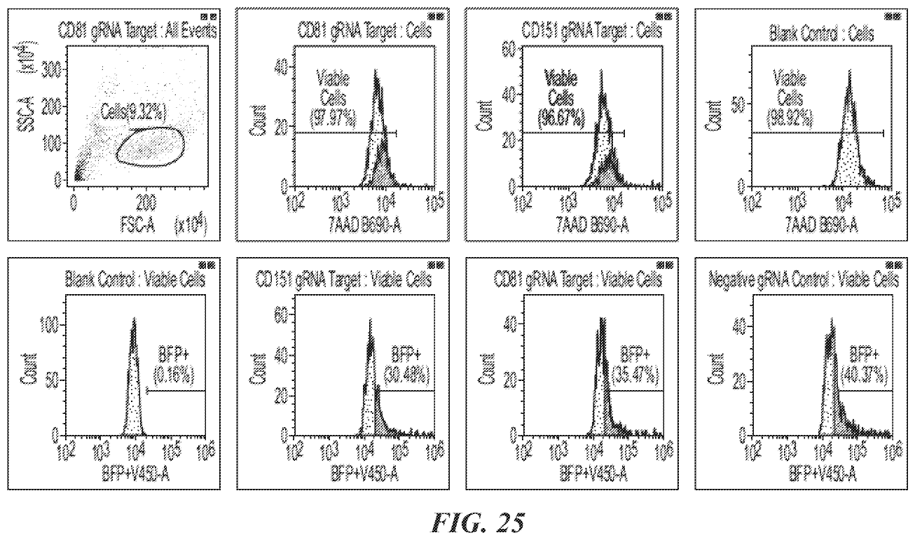

Transfected cells (e.g. transfection with ExpOFF plasmid and CD151 or CD81 targeting sgRNAs or non-targeting control) were sorted 72 hours after transfections via a BFP protein fused on the ExpOFF protein for positive gating. Sorted cells were passaged every 2-3 days based on confluency. Flow analysis was conducted using a Beckman Coulter Cytoflex and cell sorting was conducted using a Beckman Coulter Cytoflex SRT. Antibodies that were utilized for staining included PE anti-human CD151 (CAT #350408) and APC anti-human CD81 (CAT #349510). Cell staining was conducted via incubation with antibodies at 4 C for 30 minutes in PBS with 1% FBS. Cells are then washed and stained with a viability stain. Viability staining was performed for all experiments using eBioscience 7-AAD Viability Staining Solution (CAT #00-6993-50). As shown in FIG. 25, FSC-A and SSC-A gating and viability by 7AA exclusion was used for gating strategy. Furthermore, FACS was gated for BFP expression cells transfected with ExpOFF plasmid and CD151 or CD81 targeting sgRNAs, or non-targeting sgRNA control to yield an enriched population of successfully transfected cells. These cells were cultured and expanded (e.g., passaged every 2-3 days based on confluency) until enough total cells were present for flow analysis.

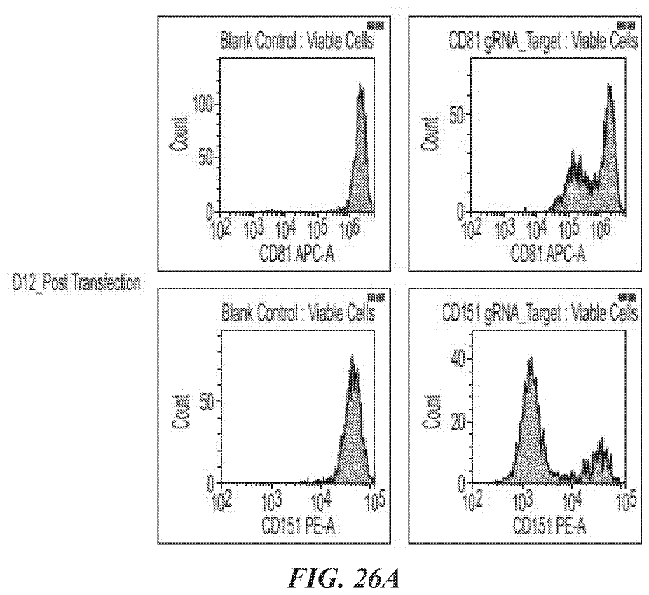

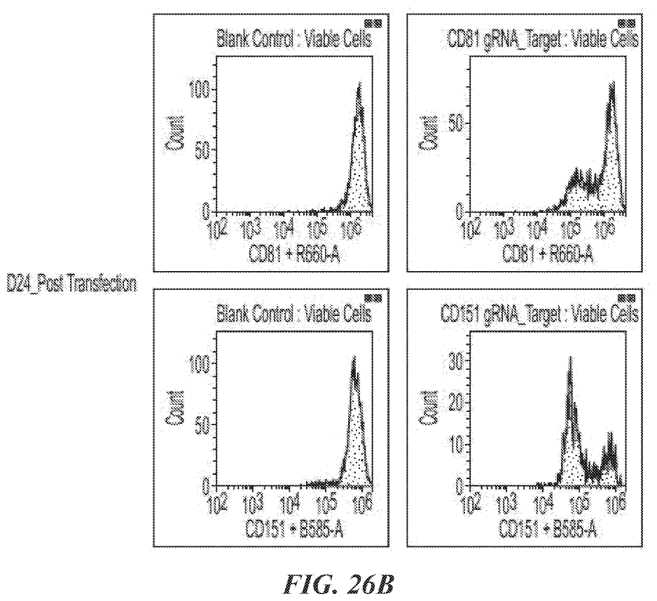

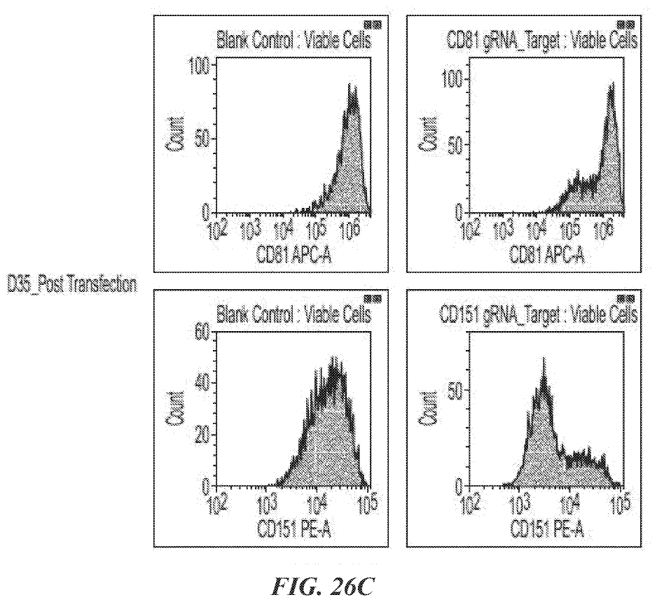

13 days after flow sorting, the sorted samples were stained with anti-CD151 and anti-CD81 antibodies and underwent flow analysis to profile if methylation has occurred at the targeted sites as shown in FIG. 26A-26C. Successful methylation was inferred upon downregulation of protein expression. In samples transfected with gene-targeting sgRNAs, their cognate targets were observed to be down-regulated compared to the blank control. The signature was retained at 12 days (FIG. 26A), at days 24 (FIG. 26B), and 35 days (FIG. 26C) after transfection, suggesting that the methylation can be retained for several days. The non-targeting sgRNA negative control sample did not recover post sorting and the sample could not be cultured further for analysis; however, the blank control was utilized as a substitute negative control.

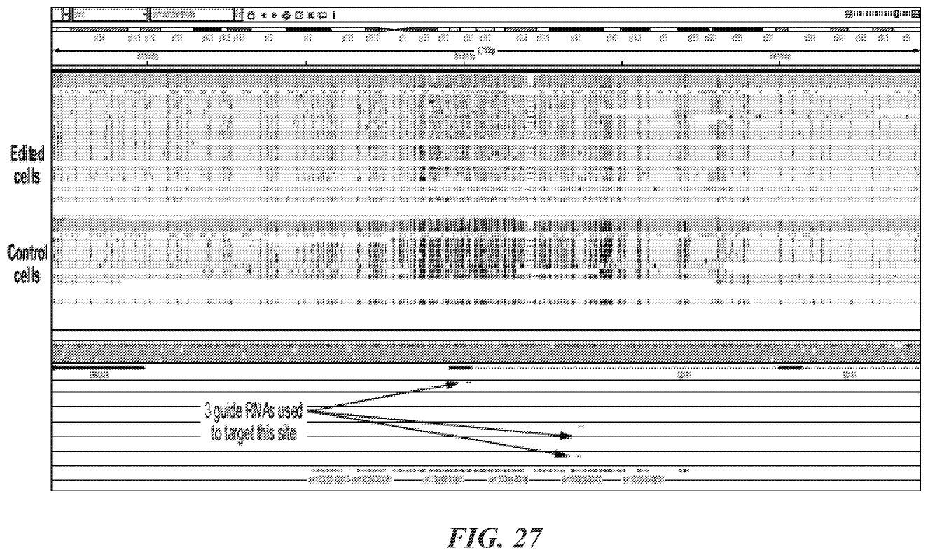

Following 3 months of culture, cells from the samples transfected with the CD151 gene-targeting sgRNAs and cells from a control sample (with dCas9 and DNMT3A only and no sgRNAs) were sequenced using read methylation sequencing and the DNA methylation patterns were analyzed by generating epigenetic maps from the sequencing data. FIG. 27 shows epigenetic maps of chromosome 11 (positions 831, 698-834, 439), depicting the methylation patterns in the CD151 gene of the edited cells and of the control cells. The epigenetic maps show a differential in methylation patterns between the edited cells and the control cells. Importantly, the targeted site in the CD151 promoter region is methylated (indicate by light gray lines) in the edited cells and unmethylated (indicated by dark gray lines) in the control cells.

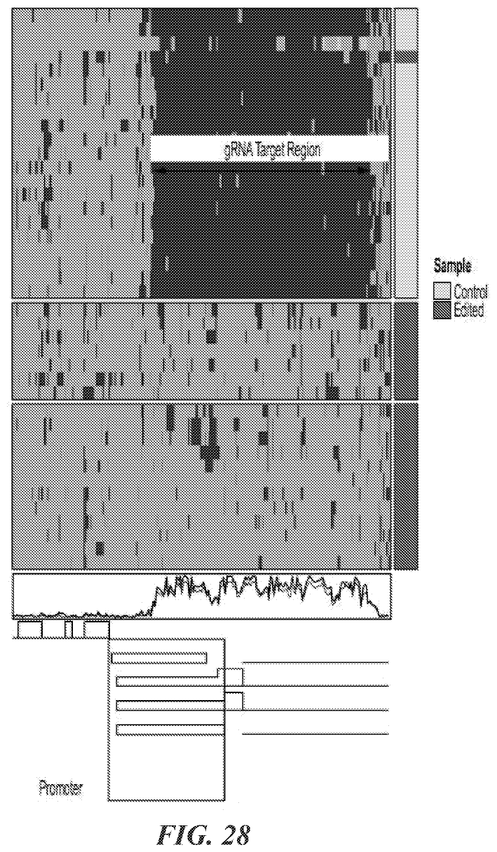

Epigenetic maps of the edited cells and the control cells were further generated using unsupervised clustering of epigenetic states, as further described in Example 3. FIG. 28 shows the epigenetic maps generated for the edited cells and the control cells, indicating differentially methylated regions. In FIG. 28, the dark gray regions represent unmethylated regions and the light gray regions represent methylated regions. The epigenetic maps indicate a region that is substantially unmethylated for the control cells but are substantially methylated for the edited cells.

This example indicates that CRISPR epigenetic systems can introduce epigenetic modifications to target sites, specified by an associated guide RNA sequence, as shown by the targeted methylation of the CD151 promoter in HEK293S cells.

The methods described in this example can further be used to screen various CRISPR epigenetic systems and guide RNAs for their ability to edit the desired target sites and refine epigenetic editing to reduce editing of off-target DNA sites. For example, multiple sgRNAs can be screened using these methods and the epigenetic editing can be iteratively improved through improving; guide designs to be more accurate/specific for the target site.

TABLE 5
Details of Transfection Setup

| | | sgRNA(50 nM | |
|---|---|---|---|
| Sample | | final | |
| Condition | Plasmid | concentration) | Seed Volume(mL) |
| Control | 10 ug | N/A | 2 |
| | of ExpOFF | | |
| CD81 | 10 ug | CD81_sgRNA 1 | 2 |
| Target | of ExpOFF | CD81_sgRNA 2 | |
| | | CD81_sgRNA 3 | |
| CD151 | 10 ug | CD151_sgRNA 1 | 2 |
| Target | of ExpOFF | CD151_sgRNA 2 | |
| | | CD151_sgRNA 3 | |
| Negative | 10 ug | NC_sgRNA 1 | 2 |
| Control | of ExpOFF | NC_sgRNA 2 | |
| Target | | NC_sgRNA 3 | |
| GFP_mRNA | 10 ug | N/A | 2 |
| Positive | of GFP | | |
| Control | mRNA | | |

TABLE 6
ExpOFF and CD151/CD81 sgRNA sequences

| | SEQ ID | |
|---|---|---|
| Name | NO. | Sequences |
| CD151_ | 1 | CCGGACUCGGACGCGUGGU |
| sgRNA 1 | | |
| CD151_ | 2 | UGUCCAGGGACAAUGAGCA |
| sgRNA 2 | | |
| CD151_ | 3 | GACAAUGAGCAGGGUGUCC |
| sgRNA 3 | | |
| CD81_ | 4 | GAGAGCGAGCGCGCAACGG |
| sgRNA 1 | | |
| CD81_ | 5 | GGCCUGGCAGGAUGCGCGG |
| sgRNA 2 | | |
| CD81_ | 6 | CCGAAACGCGCCAAGUUGG |
| sgRNA 3 | | |
| ExpOff_ | 15 | GUUUUAGAGCUAGAAAUAGCAAGUUAAAAUAAGGCUAGU |
| sgRNA | | CCGUUAUCAACUUGAAAAAGUGGCACCGAGUCGGUGCUUU |
| scaffold | | UU |
| ExpOff_ | 8 | CCGGACUCGGACGCGUGGUGUUUUAGAGCUAGAAAUAGCA |
| CD151_ | | AGUUAAAAUAAGGCUAGUCCGUUAUCAACUUGAAAAAGU |
| sgRNA 1 | | GGCACCGAGUCGGUGCUUUUU |
| ExpOff_ | 9 | UGUCCAGGGACAAUGAGCAGUUUUAGAGCUAGAAAUAGC |
| CD151_ | | AAGUUAAAAUAAGGCUAGUCCGUUAUCAACUUGAAAAAG |
| sgRNA 2 | | UGGCACCGAGUCGGUGCUUUUU |
| ExpOff_ | 10 | GACAAUGAGCAGGGUGUCCGUUUUAGAGCUAGAAAUAGC |
| CD151_ | | AAGUUAAAAUAAGGCUAGUCCGUUAUCAACUUGAAAAAG |
| sgRNA 3 | | UGGCACCGAGUCGGUGCUUUUU |
| ExpOff_ | 11 | GAGAGCGAGCGCGCAACGGGUUUUAGAGCUAGAAAUAGC |
| CD81_ | | AAGUUAAAAUAAGGCUAGUCCGUUAUCAACUUGAAAAAG |
| sgRNA 1 | | UGGCACCGAGUCGGUGCUUUUU |
| ExpOff_ | 12 | GGCCUGGCAGGAUGCGCGGGUUUUAGAGCUAGAAAUAGC |
| CD81_ | | AAGUUAAAAUAAGGCUAGUCCGUUAUCAACUUGAAAAAG |
| sgRNA 2 | | UGGCACCGAGUCGGUGCUUUUU |
| ExpOff_ | 13 | CCGAAACGCGCCAAGUUGGGUUUUAGAGCUAGAAAUAGCA |
| CD81_ | | AGUUAAAAUAAGGCUAGUCCGUUAUCAACUUGAAAAAGU |
| sgRNA 3 | | GGCACCGAGUCGGUGCUUUUU |
| ExpOFF | 7 | ATGAACCACGACCAAGAGTTCGACCCGCCTAAGGTGTACCC |
| DNA | | TCCTGTGCCTGCCGAGAAGCGGAAGCCTATCAGAGTGCTGA |
| sequence | | GCCTGTTCGACGGAATCGCCACAGGACTGCTGGTGCTGAAG |
| | | GATCTGGGAATCCAGGTGGACCGGTATATCGCCAGCGAAGT |
| | | GTGCGAGGACAGCATCACAGTTGGCATGGTTCGACACCAGG |
| | | GCAAGATTATGTACGTGGGCGACGTGCGGAGCGTGACCCAG |
| | | AAACACATTCAAGAGTGGGGCCCCTTCGACCTGGTTATCGG |
| | | CGGCAGCCCTTGCAACGATCTGAGCATCGTGAACCCCGCCA |
| | | GAAAGGGCCTGTATGAAGGCACCGGCAGACTGTTCTTCGAG |
| | | TTCTACAGACTGCTGCACGACGCCAGACCTAAAGAGGGCGA |
| | | CGACAGACCATTCTTCTGGCTGTTCGAGAACGTGGTGGCCAT |
| | | GGGCGTGTCCGACAAGCGGGATATCAGCCGGTTCCTGGAAA |
| | | GCAACCCCGTGATGATCGACGCCAAAGAGGTGTCCGCTGCT |
| | | CACCGGGCCAGATACTTTTGGGGCAATCTGCCCGGCATGAA |
| | | CAGACCTCTGGCCAGCACCGTGAACGACAAGCTGGAACTGC |
| | | AAGAGTGCCTGGAACACGGCCGGATCGCCAAGTTTAGCAAA |
| | | GTGCGGACCATCACCACCAGGTCCAACAGCATCAAGCAGGG |
| | | CAAAGACCAGCACTTCCCCGTGTTCATGAATGAGAAAGAGG |
| | | ACATCCTGTGGTGCACCGAGATGGAAAGAGTGTTCGGCTTC |
| | | CCTGTGCACTACACCGACGTGTCCAACATGAGCAGACTGGC |
| | | CAGACAGAGGCTGCTGGGCAGATCTTGGAGCGTGCCAGTGA |
| | | TCAGACACCTGTTCGCCCCTCTGAAAGAATACTTCGCCTGCG |
| | | TGTCCAGCGGCAACAGCAACGCCAATTCTAGAGGCCCCAGC |
| | | TTCAGCTCTGGACTGGTGCCATTGAGCCTGAGAGGCTCTCAC |
| | | ATGGGCCCCATGGAAATCTACAAGACCGTGTCCGCCTGGAA |
| | | GAGACAGCCTGTCAGAGTCCTGTCTCTGTTCCGCAACATCG |
| | | ACAAGGTGCTGAAAAGCCTGGGCTTCCTGGAATCTGGCAGC |
| | | GGATCTGGCGGCGGAACCCTGAAATACGTGGAAGATGTGAC |
| | | CAACGTCGTGCGGCGCGACGTGGAAAAGTGGGGACCTTTTG |
| | | ATCTGGTGTACGGCAGCACACAGCCACTGGGCAGCAGCTGC |
| | | GATAGATGTCCTGGCTGGTACATGTTCCAGTTCCACCGGATC |
| | | CTGCAGTACGCCCTGCCTAGACAAGAGAGCCAGCGGCCTTT |
| | | CTTCTGGATCTTCATGGACAACCTGCTGCTGACCGAGGACG |
| | | ATCAAGAGACAACCACCAGATTTCTGCAGACCGAGGCCGTG |
| | | ACACTCCAGGATGTTCGGGGCAGAGACTACCAGAACGCCAT |
| | | GAGAGTGTGGTCTAACATCCCCGGCCTGAAGTCCAAACACG |
| | | CCCCACTGACACCCAAAGAGGAAGAGTACCTGCAGGCCCAA |
| | | GTGCGGAGCAGATCCAAACTGGATGCCCCTAAGGTGGACCT |
| | | GCTGGTCAAGAACTGTCTGCTGCCCCTGAGAGAGTACTTCA |
| | | AGTATTTCAGCCAGAACAGCCTGCCTCTCGGCGGACCTTCTA |
| | | GCGGAGCACCTCCTCCAAGCGGAGGATCTCCTGCTGGAAGC |
| | | CCTACCTCTACCGAGGAAGGCACAAGCGAGTCTGCCACACC |
| | | TGAGTCTGGCCCTGGCACATCTACAGAGCCTAGCGAAGGTT |
| | | CTGCCCCTGGATCTCCAGCCGGCTCTCCTACATCTACTGAAG |
| | | AGGGCACCAGCACCGAGCCATCTGAAGGCAGTGCACCTGGC |
| | | ACAAGCACAGAGCCCTCCGAAATGGACAAGAAGTACAGCA |
| | | TCGGCCTGGCCATCGGCACCAATTCTGTTGGATGGGCCGTG |
| | | ATCACCGACGAGTACAAGGTGCCCAGCAAGAAGTTCAAGGT |
| | | CCTGGGCAACACCGACCGGCACTCCATCAAGAAGAATCTGA |
| | | TCGGCGCCCTGCTGTTCGACTCTGGCGAAACAGCCGAAGCC |
| | | ACCAGACTGAAGAGAACCGCCAGAAGGCGCTACACCCGGC |
| | | GGAAGAACCGGATCTGTTACCTGCAAGAGATCTTCAGCAAC |
| | | GAGATGGCCAAGGTGGACGACAGCTTCTTCCACAGACTGGA |
| | | AGAGTCCTTCCTGGTGGAAGAGGATAAGAAGCACGAGCGGC |
| | | ACCCCATCTTCGGCAACATCGTGGATGAGGTGGCCTACCAC |
| | | GAGAAGTACCCCACCATCTACCACCTGAGAAAGAAACTGGT |
| | | GGACAGCACCGACAAGGCCGACCTGAGACTGATCTATCTGG |
| | | CTCTGGCCCACATGATCAAGTTCCGGGGCCACTTTCTGATCG |
| | | AGGGCGATCTGAACCCCGACAACTCCGACGTGGACAAGCTG |
| | | TTCATCCAGCTGGTGCAGACCTACAACCAGCTGTTCGAAGA |
| | | GAATCCCATCAACGCCTCTGGCGTGGACGCCAAGGCTATCC |
| | | TGTCTGCCAGACTGAGCAAGAGCAGACGGCTGGAAAACCTG |
| | | ATCGCTCAGCTGCCAGGCGAGAAGAAGAACGGCCTGTTCGG |
| | | CAACCTGATTGCCCTGAGCCTGGGCCTGACACCTAACTTCA |
| | | AGAGCAACTTCGATCTGGCCGAGGATGCCAAACTGCAGCTG |
| | | TCCAAGGACACCTACGACGACGACCTGGATAATCTGCTGGC |
| | | CCAGATCGGCGACCAGTACGCCGACTTGTTTCTGGCCGCCA |
| | | AGAACCTGTCCGACGCCATCCTGCTGAGCGACATTCTGAGA |
| | | GTGAACACCGAGATCACCAAGGCACCTCTGAGCGCCAGCAT |
| | | GATTAAGAGATACGACGAGCACCATCAGGATCTGACCCTGC |
| | | TGAAGGCCCTCGTCAGACAGCAGCTCCCAGAGAAGTACAAA |
| | | GAGATTTTCTTCGACCAGAGCAAGAACGGCTACGCCGGCTA |
| | | CATTGATGGCGGAGCCAGCCAAGAGGAATTCTACAAGTTCA |
| | | TCAAGCCCATCCTCGAGAAGATGGACGGCACCGAGGAACTG |
| | | CTCGTGAAGCTGAACAGAGAGGATCTGCTGAGAAAGCAGA |
| | | GGACCTTCGACAACGGCAGCATCCCTCACCAGATCCACCTG |
| | | GGAGAACTGCACGCCATTCTGCGGAGACAAGAGGACTTTTA |
| | | CCCATTCCTGAAGGACAACCGGGAAAAGATTGAGAAGATCC |
| | | TGACCTTCAGGATCCCCTACTACGTGGGACCACTGGCCAGG |
| | | GGCAATAGCAGATTTGCCTGGATGACCAGAAAGAGCGAGG |
| | | AAACCATCACACCCTGGAACTTCGAGGAAGTGGTGGATAAG |
| | | GGCGCCAGCGCTCAGTCCTTCATCGAGCGGATGACCAACTT |
| | | CGACAAGAATCTGCCTAACGAGAAGGTGCTGCCCAAGCACA |
| | | GCCTGCTGTACGAGTACTTTACCGTGTACAACGAGCTGACC |
| | | AAAGTCAAATACGTGACCGAGGGGATGAGAAAGCCCGCCTT |
| | | TCTGAGCGGCGAGCAGAAAAAGGCCATCGTCGATCTGCTGT |
| | | TCAAGACCAACCGGAAAGTGACCGTGAAGCAGCTGAAAGA |
| | | GGATTACTTCAAGAAAATCGAGTGCTTCGACAGCGTGGAAA |
| | | TCAGCGGCGTCGAGGACAGATTCAATGCCAGCCTGGGCACA |
| | | TACCACGACCTGCTGAAAATTATCAAGGACAAGGACTTCCT |
| | | GGACAACGAGGAAAACGAGGACATTCTTGAGGACATCGTGC |
| | | TGACCCTGACACTGTTCGAGGATAGAGAGATGATCGAGGAA |
| | | CGGCTGAAAACATACGCCCACCTGTTTGACGACAAAGTGAT |
| | | GAAGCAACTGAAGCGGCGGAGATACACCGGCTGGGGTAGA |
| | | CTGTCCCGGAAGCTGATCAACGGCATCCGGGATAAGCAGTC |
| | | CGGCAAGACCATCCTGGACTTTCTGAAGTCCGACGGCTTCG |
| | | CCAACAGAAACTTCATGCAGCTGATCCACGACGACAGCCTG |
| | | ACCTTTAAAGAGGATATCCAGAAGGCCCAGGTTTCCGGCCA |
| | | GGGCGATTCTCTGCACGAGCACATTGCCAATCTGGCCGGAT |
| | | CTCCCGCCATTAAGAAGGGCATCCTGCAGACAGTGAAGGTG |
| | | GTGGATGAGCTGGTCAAAGTCATGGGCAGACACAAGCCCGA |
| | | GAACATCGTGATCGAAATGGCCAGAGAGAACCAGACCACA |
| | | CAGAAGGGACAGAAGAACAGCCGCGAGAGAATGAAGCGGA |
| | | TCGAAGAGGGAATCAAAGAGCTGGGCAGCCAGATCCTCAA |
| | | AGAACACCCCGTGGAAAACACCCAGCTGCAGAACGAGAAG |
| | | CTGTACCTGTACTACCTGCAGAATGGACGGGATATGTACGTT |
| | | GACCAAGAGCTGGACATCAACCGGCTGTCCGACTACGATGT |
| | | GGACGCTATCGTGCCCCAGAGCTTCCTGAAAGACGACTCCA |
| | | TCGACAACAAAGTGCTGACTCGGAGCGACAAGAACCGGGG |
| | | CAAGAGCGACAATGTGCCAAGCGAAGAGGTCGTGAAGAAG |
| | | ATGAAGAACTACTGGCGACAGCTGCTGAATGCCAAGCTGAT |
| | | TACCCAGCGGAAGTTCGACAATCTGACCAAGGCCGAGAGAG |
| | | GCGGCCTGAGCGAACTTGATAAGGCCGGCTTCATTAAGCGG |
| | | CAGCTGGTGGAAACCCGGCAGATCACAAAACACGTGGCAC |
| | | AGATCCTGGATAGCCGGATGAACACTAAGTACGACGAGAAC |
| | | GACAAACTGATCCGGGAAGTGAAAGTGATCACCCTCAAGTC |
| | | CAAGCTGGTGTCCGATTTCCGGAAGGATTTCCAGTTCTACAA |
| | | AGTCCGCGAGATCAACAACTACCATCACGCCCACGACGCCT |
| | | ACCTGAATGCCGTTGTTGGAACAGCCCTGATCAAGAAGTAT |
| | | CCCAAGCTCGAGAGCGAGTTCGTGTACGGCGACTACAAGGT |
| | | GTACGATGTGCGCAAGATGATTGCCAAGAGCGAGCAAGAA |
| | | ATCGGCAAGGCTACCGCCAAGTACTTCTTCTACAGCAACAT |
| | | CATGAATTTCTTCAAGACAGAGATCACGCTGGCCAACGGCG |
| | | AGATCCGGAAAAGACCCCTGATCGAGACAAACGGCGAAAC |
| | | CGGGGAGATCGTGTGGGACAAGGGCAGAGATTTTGCCACCG |
| | | TGCGGAAAGTGCTGTCTATGCCTCAAGTGAACATCGTCAAA |
| | | AAGACCGAGGTGCAGACAGGCGGCTTCAGCAAAGAGTCTAT |
| | | CCTGCCTAAGAGGAACAGCGATAAGCTGATCGCCCGGAAGA |
| | | AGGACTGGGACCCTAAGAAGTACGGCGGCTTCGATAGCCCT |
| | | ACCGTGGCCTATTCTGTGCTGGTGGTGGCAAAGGTGGAAAA |
| | | GGGCAAGTCCAAAAAGCTCAAGAGCGTGAAAGAGCTGCTG |
| | | GGGATCACCATCATGGAAAGAAGCAGCTTCGAGAAGAACC |
| | | CGATCGACTTTCTGGAAGCCAAGGGCTACAAAGAAGTCAAG |
| | | AAGGACCTGATCATCAAGCTCCCCAAGTACTCCCTGTTCGA |
| | | GCTGGAAAATGGCCGGAAGCGGATGCTGGCTTCTGCTGGCG |
| | | AACTGCAGAAGGGAAACGAACTGGCCCTGCCTTCCAAATAT |
| | | GTGAACTTCCTGTACCTGGCCAGCCACTATGAGAAGCTGAA |
| | | GGGCAGCCCCGAGGACAATGAGCAAAAGCAGCTGTTTGTGG |
| | | AACAGCACAAACACTACCTGGACGAGATCATCGAGCAGATC |
| | | AGCGAGTTCTCCAAGAGAGTGATCCTGGCCGACGCTAACCT |
| | | GGACAAAGTCCTGAGCGCCTACAACAAGCACCGGGACAAG |
| | | CCAATCAGAGAGCAGGCCGAGAATATCATCCACCTGTTCAC |
| | | CCTGACCAACCTGGGAGCCCCTGCCGCCTTCAAGTACTTTGA |
| | | TACCACCATCGACCGGAAGAGGTACACCTCCACAAAAGAGG |
| | | TGCTGGACGCCACACTGATCCACCAGTCTATCACCGGCCTGT |
| | | ACGAGACACGGATCGACCTGTCTCAGCTTGGAGGCGACGCC |
| | | TATCCTTACGACGTGCCAGACTATGCTAGCCTCGGCTCTGGC |
| | | AGCCCCAAGAAGAAGAGAAAGGTCGAGGACCCCAAGAAAA |
| | | AGCGCAAAGTGGACGGCATCGGCAGCGGCTCTAATGGCTCT |
| | | AGCGGAAGCAGCGAACTGATCAAAGAAAACATGCACATGA |
| | | AGCTCTACATGGAAGGCACTGTGGACAATCACCACTTCAAG |
| | | TGCACCAGCGAAGGCGAGGGCAAGCCTTATGAGGGCACCC |
| | | AGACCATGCGGATCAAGGTGGTCGAAGGCGGACCTCTGCCT |
| | | TTCGCCTTCGATATCCTGGCTACCAGCTTTCTGTACGGCTCC |
| | | AAGACCTTCATCAATCACACCCAGGGCATCCCCGACTTTTTC |
| | | AAGCAGAGCTTTCCCGAGGGCTTCACTTGGGAGAGAGTGAC |
| | | CACATACGAGGACGGCGGAGTGCTGACAGCCACACAGGAT |
| | | ACAAGCCTGCAGGACGGCTGCCTGATCTACAACGTGAAGAT |
| | | CCGGGGCGTGAACTTCACCAGCAACGGCCCTGTGATGCAGA |
| | | AGAAAACCCTCGGCTGGGAAGCCTTCACCGAGACACTGTAT |
| | | CCAGCCGATGGCGGACTGGAAGGCCGGAATGATATGGCCCT |
| | | GAAGCTCGTTGGCGGCTCCCATCTGATCGCCAACATCAAGA |
| | | CCACCTACAGAAGCAAGAAGCCCGCTAAGAACCTCAAGATG |
| | | CCCGGCGTGTACTATGTGGACTACCGGCTGGAACGCATCAA |
| | | AGAGGCCAACAACGAAACCTACGTCGAGCAGCACGAAGTG |
| | | GCCGTGGCCAGATATTGCGACCTGCCTTCTAAGCTGGGCCA |
| | | CAAGTTGAATGGCGGAGGCGGAGGAATGGACGCTAAGAGC |
| | | CTTACCGCCTGGTCTAGAACCCTGGTCACCTTCAAGGACGTG |
| | | TTCGTGGACTTCACCCGGGAAGAGTGGAAGCTGCTGGACAC |
| | | AGCACAGCAGATTGTGTACCGGAACGTGATGCTCGAGAACT |
| | | ACAAAAACCTGGTGTCCCTGGGCTATCAGCTGACAAAGCCC |
| | | GACGTGATCCTGCGCCTGGAAAAAGGCGAAGAACCCTAG |
| ExpOFF | 14 | MNHDQEFDPPKVYPPVPAEKRKPIRVLSLFDGIATGLLVLKDL |
| Protein | | GIQVDRYIASEVCEDSITVGMVRHQGKIMYVGDVRSVTQKHIQ |
| Sequence | | EWGPFDLVIGGSPCNDLSIVNPARKGLYEGTGRLFFEFYRLLHD |
| | | ARPKEGDDRPFFWLFENVVAMGVSDKRDISRFLESNPVMIDAK |
| | | EVSAAHRARYFWGNLPGMNRPLASTVNDKLELQECLEHGRIA |
| | | KFSKVRTITTRSNSIKQGKDQHFPVFMNEKEDILWCTEMERVF |
| | | GFPVHYTDVSNMSRLARQRLLGRSWSVPVIRHLFAPLKEYFAC |
| | | VSSGNSNANSRGPSFSSGLVPLSLRGSHMGPMEIYKTVSAWKR |
| | | QPVRVLSLFRNIDKVLKSLGFLESGSGSGGGTLKYVEDVTNVV |
| | | RRDVEKWGPFDLVYGSTQPLGSSCDRCPGWYMFQFHRILQYA |
| | | LPRQESQRPFFWIFMDNLLLTEDDQETTTRFLQTEAVTLQDVR |
| | | GRDYQNAMRVWSNIPGLKSKHAPLTPKEEEYLQAQVRSRSKL |
| | | DAPKVDLLVKNCLLPLREYFKYFSQNSLPLGGPSSGAPPPSGGS |
| | | PAGSPTSTEEGTSESATPESGPGTSTEPSEGSAPGSPAGSPTSTEE |
| | | GTSTEPSEGSAPGTSTEPSEMDKKYSIGLAIGTNSVGWAVITDE |
| | | YKVPSKKFKVLGNTDRHSIKKNLIGALLFDSGETAEATRLKRT |
| | | ARRRYTRRKNRICYLQEIFSNEMAKVDDSFFHRLEESFLVEEDK |
| | | KHERHPIFGNIVDEVAYHEKYPTIYHLRKKLVDSTDKADLRLIY |
| | | LALAHMIKFRGHFLIEGDLNPDNSDVDKLFIQLVQTYNQLFEEN |
| | | PINASGVDAKAILSARLSKSRRLENLIAQLPGEKKNGLFGNLIAL |
| | | SLGLTPNFKSNFDLAEDAKLQLSKDTYDDDLDNLLAQIGDQYA |
| | | DLFLAAKNLSDAILLSDILRVNTEITKAPLSASMIKRYDEHHQD |
| | | LTLLKALVRQQLPEKYKEIFFDQSKNGYAGYIDGGASQEEFYK |
| | | FIKPILEKMDGTEELLVKLNREDLLRKQRTFDNGSIPHQIHLGEL |
| | | HAILRRQEDFYPFLKDNREKIEKILTFRIPYYVGPLARGNSRFA |
| | | WMTRKSEETITPWNFEEVVDKGASAQSFIERMTNFDKNLPNEK |
| | | VLPKHSLLYEYFTVYNELTKVKYVTEGMRKPAFLSGEQKKAIV |
| | | DLLFKTNRKVTVKQLKEDYFKKIECFDSVEISGVEDRFNASLGT |
| | | YHDLLKIIKDKDFLDNEENEDILEDIVLTLTLFEDREMIEERLKT |
| | | YAHLFDDKVMKQLKRRRYTGWGRLSRKLINGIRDKQSGKTIL |
| | | DFLKSDGFANRNFMQLIHDDSLTFKEDIQKAQVSGQGDSLHEH |
| | | IANLAGSPAIKKGILQTVKVVDELVKVMGRHKPENIVIEMARE |
| | | NOTTQKGQKNSRERMKRIEEGIKELGSQILKEHPVENTQLQNE |
| | | KLYLYYLQNGRDMYVDQELDINRLSDYDVDAIVPQSFLKDDSI |
| | | DNKVLTRSDKNRGKSDNVPSEEVVKKMKNYWRQLLNAKLIT |
| | | QRKFDNLTKAERGGLSELDKAGFIKRQLVETRQITKHVAQILDS |
| | | RMNTKYDENDKLIREVKVITLKSKLVSDFRKDFQFYKVREINN |
| | | YHHAHDAYLNAVVGTALIKKYPKLESEFVYGDYKVYDVRKMI |
| | | AKSEQEIGKATAKYFFYSNIMNFFKTEITLANGEIRKRPLIETNG |
| | | ETGEIVWDKGRDFATVRKVLSMPQVNIVKKTEVQTGGFSKESI |
| | | LPKRNSDKLIARKKDWDPKKYGGFDSPTVAYSVLVVAKVEKG |
| | | KSKKLKSVKELLGITIMERSSFEKNPIDFLEAKGYKEVKKDLIIK |
| | | LPKYSLFELENGRKRMLASAGELQKGNELALPSKYVNFLYLAS |
| | | HYEKLKGSPEDNEQKQLFVEQHKHYLDEIIEQISEFSKRVILAD |
| | | ANLDKVLSAYNKHRDKPIREQAENIIHLFTLTNLGAPAAFKYFD |
| | | TTIDRKRYTSTKEVLDATLIHQSITGLYETRIDLSQLGGDAYPY |
| | | DVPDYASLGSGSPKKKRKVEDPKKKRKVDGIGSGSNGSSGSSE |
| | | LIKENMHMKLYMEGTVDNHHFKCTSEGEGKPYEGTQTMRIKV |
| | | VEGGPLPFAFDILATSFLYGSKTFINHTQGIPDFFKQSFPEGFTW |
| | | ERVTTYEDGGVLTATQDTSLQDGCLIYNVKIRGVNFTSNGPVM |
| | | QKKTLGWEAFTETLYPADGGLEGRNDMALKLVGGSHLIANIK |
| | | TTYRSKKPAKNLKMPGVYYVDYRLERIKEANNETYVEQHEVA |
| | | VARYCDLPSKLGHKLNGGGGGMDAKSLTAWSRTLVTFKDVF |
| | | VDFTREEWKLLDTAQQIVYRNVMLENYKNLVSLGYQLTKPDV |
| | | ILRLEKGEEP |

### Example 8: Identifying Off-Target Genomic Sites for Blocking During CRISPR-Guided Epigenetic Editing

This example demonstrates an application of epigenetic mapping to analyze the effects of a CRISPR epigenetic editing system across the epigenome and the location of the modifications. This method of analysis can be useful to locate unintended modifications at off-target sites and contribute to designing approaches to minimize unintended modifications, such as selectively blocking off-target sites during CRISPR-guided epigenetic editing to block those sites from being modified. Unintended modifications can result from direct off-target editing by the CRISPR-guided epigenetic editing system or from a long-range effect from an epigenetic edit by the CRISPR-guided epigenetic editing system (e.g., by modulating a signaling pathway).

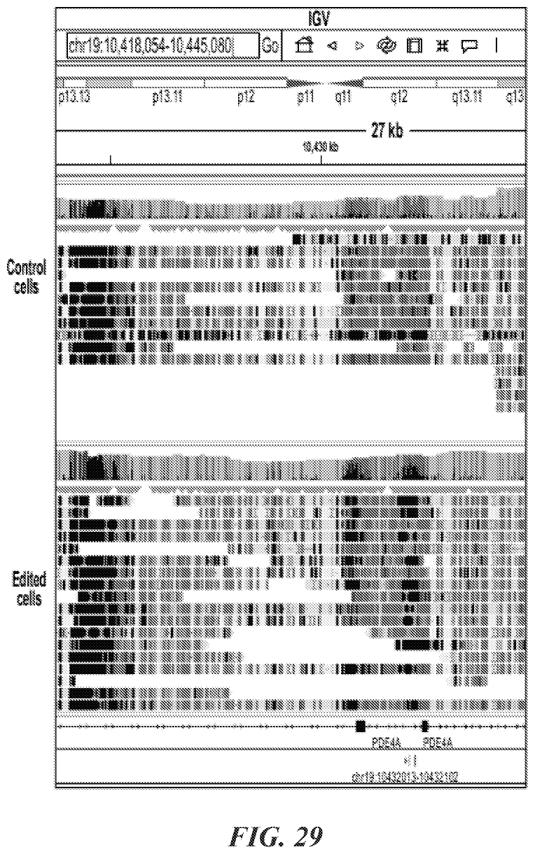

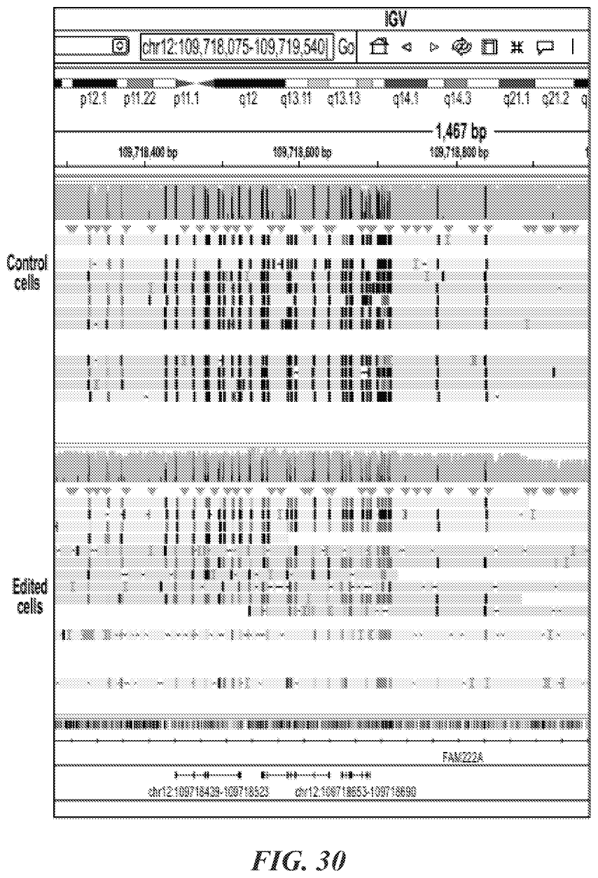

In this example, following epigenetic editing of CD151 using the 3 sgRNAs targeting CD151 shown in Table 6 and methylation sequencing, epigenetic maps were generated for other parts of the genome to analyze differentially methylated regions between the control cells and the edited cells in other parts of the genome that were not targeted by the 3 sgRNAs. FIGS. 29 and 30 are example epigenetic maps that were generated that show differentially methylated regions (light gray representing methylated regions and dark gray representing unmethylated regions) between the control cells and the edited cells in regions of chromosome 19 (FIG. 29) and chromosome 12 (FIG. 30). Some of these differentially methylated regions may be a result of direct off-target editing by the CRISPR epigenetic editing system. Others may be a result of a signaling pathway modulation resulting from a change in expression of CD151.

Analyzing the locations of the off-target modifications can be used to refine editing methods by designing selective blockers that can be incorporated during CRISPR-guided epigenetic editing to block important off-target sites from epigenetic editing. A method of selectively blocking an off-target site while simultaneously editing a target site is using combinations of orthogonal Cas systems (or Cas systems that do not cross-react), wherein one or more orthogonal Cas systems can be used to selectively block one or more off-target sites (using guide IRAs that guide the respective Cas protein(s) to bind to the off-target sites, thereby blocking epigenetic modifications), while another orthogonal Cas system introduces an epigenetic modification to a specific target site. In this example, epigenetic mapping was used to identify the location of off-target modifications in chromosome 19 and chromosome 12 resulting from the CRISPR-guided epigenetic editing of CD151. Guide RNAs for an orthogonal Cas system comprising a catalytically inactive orthogonal Cas protein can be designed to selectively block those sites of interest via binding. Such an orthogonal Cas system targeting the off-target sites for binding can be used together with the same ExpOFF epigenetic editing system targeting CD151 for methylation to refine epigenetic editing.

### Example 9: Construction of the Epigenome from DNA Fragments of Blood Samples

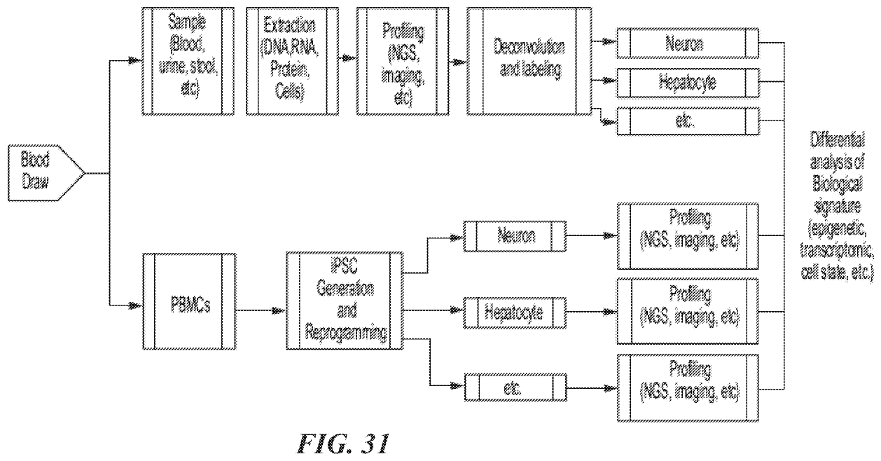

A method where no biopsies are required to construct the epigenome of an individual's cells or tissues is developed, as shown in FIG. 31. As shown in the top arm of FIG. 31, various samples, gathered without biopsy, can be collected. cfDNA or cells in these samples are profiled to extract epigenetic signatures as well as assigned to a tissue of origin. This can provide a current view of the epigenetic status of various tissues in the body.

Thus, rather than obtaining biopsies tissue samples (e.g., liver), an individual's blood was drawn to map methylation (CpG) sites in the genome. Blood samples from 23 healthy individuals were obtained. Whole blood is collected in Streak or EDTA tubes (e.g., 10 mL). Next plasma is extracted by spinning the whole blood tubes at 1500×g for 10 minutes at 20° C. at an acceleration and deceleration at 20% of maximum. The plasma layer is aseptically pipetted into a labeled 15 ml conical tube without disturbing the buffy coat and red blood cell layer. The plasma is spun at 16000×g for 10 minutes at 20° C. at an acceleration and deceleration at 20% of maximum. 1.0 mL of the double spun plasma is aseptically pipetted into labeled 1.0 mL Matrix cryovials without disturbing the pellet. The aliquots for either stored at −80° C. for later use, or cfDNA is extracted from the plasma using a standard kit (e.g., Beckman Apostle MiniMax high efficiency cfDNA isolation kit or QIAmp circulating nucleic acid kit). Sequencing libraries for individual are prepared from the cell-free DNA and then sequenced using Illumina sequencing. Molecular deconvolution of the sequencing data from the cell free DNA library is performed.

As shown in the bottom arm of FIG. 31, in some instances, this method can be applied to iPSC-derived tissues or cell types to profile the epigenome. PMBCs collected from a blood draw can be reprogrammed into iPSC and subsequently differentiated into various tissues of interest. These tissues can then be profiled as described herein to extract epigenetic signatures. A differential analysis of the epigenetic signatures of both anns shown in FIG. 31 may provide insight into how the epigenome for a specific tissue changes relative to a common baseline (e.g., iPSC-derived epigenetic signature).

### Example 10: Single-Cell Methylome Sequencing of CD8+ T Cells in Different Differentiation States

This example shows an example of single-cell methylome sequencing of CD8+ T cells in the following differentiation states: naïve CD8+ T-cells, central memory CD8+T-cells, effector memory CD8+ T-cells, and effector CD8+ T-cells.

First, CD8+ T-cells from a donor are sorted using flow cytometry by the CD62L and CD45RO markers into the following populations: naïve CD8+ T-cell population (CD62L+/CD45RO−), the central memory CD8+T-cell population (CD62L+/CD45RO+), the effector memory CD8+ T-cell population (CD62L−/CD45RO+), and effector CD8+ T-cell population (CD62L−/CD45RO−). The cells are index sorted into an Eppendorf twin-tech, loBind 96-well plate, partitioned into wells of a) single cells, b) pools of 4 cells, and c) pools 10 cells containing 2.5 μL of lysis buffer (10 mM Tris HCl, pH8.0, 0.67 mg/mL Proteinase K and 9 μg of Unmethylated lambda DNA for single cell methylome sequencing). The sorted CD8+ cells are used to prepare single cell methylome sequencing libraries for sequencing using Illumina platforms. Each library (single-cell or mini-pool of 4 or 10 cells), requires 25-50 million reads.

### Cell Lysis

First, a volume of 7 μL of mineral oil are added to the partitioned cells. The cells are incubated at 98° C. to lyse the cells and denature the proteins. A volume of 3 μL of Single-cell Lysis buffer (10 mM Tris HCl, pH8.0, 0.67 mg/mL Proteinase K) is added to each well. ( ). The samples are gently vortexed (speed 4-5/10) and centrifuged for 5 min. at 2000 rpm at room temperature. A volume of 4.5 μL of molecular biology grade water was added for a final volume of 10 μL. The cells are incubated at 55° C. for 10 min. in a thermocycler to digest proteins.

### Bisulfite Conversion

The DNA in the cell lysate is then subjected to bisulfite conversion. The CT conversion reagent is prepared by resuspending 1 CT conversion tube with 790 μL of M-solubilization buffer and 300 μL M-dilution buffer. The CT conversion reagent is incubated at 50 C for 5-10 min. and vortexed every 30 seconds until no precipitates are visible, A volume of 160 μl of M-Reaction Buffer is added and vortexed, A volume of 65 μL of CT Conversion reagent is then added to each well of cell lysate and incubated in the thermocycler with the following program: 1) 98° C. for 8 min., 2) 65° C. for 180 min, 3) hold at 4° C.

### Desulfonation and Purification of Bi-Sulfite-Converted DNA

To desulfonate and purify the bi-sulfite-Converted DNA, the DNA is first bound to MagBinding Beads. A volume of 5 μl of MagBinding Beads is added to 300 μl of M-Binding Buffer into 96-Well 1 mL polypropylene plate. The 75 μL of bi-sulfite-converted DNA sample s transferred to the MagBinding Beads and M-Binding Buffer mixture. The wells are rinsed with 75 μL of this mixture to collect any remaining sample and combined with the MagBinding Beads and M-Binding Buffer mixture and mixed by vortexing. The mixture is incubated at room temperature for 5 min. to bind the DNA to the MagBinding Beads. The plate is centrifuged for 1 min. at 1500 rpm at RT and then placed on a magnet for 5 min. (or until the solution clears), before the supernatant s removed and discarded. The plate s removed from the magnet, and the beads were washed in 200 μL of M-wash buffer. The plate is placed on a magnet for 3 min. (or until the solution clears) and the supernatant is removed.

To desulfonate the DNA, the plate is removed from the magnet, and 100 μL of M-Desulfonation Buffer was added and mixed thoroughly. The plate is then incubated at room temperature for 15 min. The plate is placed on a magnet for 3 min. (or until the solution clears) and the supernatant is removed and discarded.

The DNA-bound beads are then washed twice with M-wash buffer. Each wash is done by removing the plate from the magnet, adding 200 μL of M-wash buffer to the beads and mixing thoroughly, placing on a magnet for 3 min. (or until the solution clears) and removing and discarding the supernatant.

Finally, the MagBinding beads are air dried by heating at 55° C. for 5 min. to evaporate residual M-wash buffer. The beads are resuspended in 40 μl of preamplification mix (1× Blue buffer, 0.4 mM dNTP Mix, and 0.4 μM preamplification primer) and incubated at 55° C. for 4 minutes to elute the DNA from the beads. The plate is placed on the magnet for 3 min. and 39 μl of the supernatant containing the DNA are transferred into a fresh PCR low binding 96-well plate.

### Preamplification, Endonuclease I+Shrimp Alkaline Phosphatase Treatment, and Purification

For preamplification of the DNA, the samples are incubated in a thermocycler at 65° C. for 3 min. and the PCR plate is transferred into a precooled aluminum rack (4° C.). The plate is centrifuged at 500×g for 10 s at 15-25° C. to collect all the liquid content in the bottom. Then, 1 μL of Klenow exo at 50 U/uL stock is added to each well.

The following steps are repeated a total of four times to perform four rounds of first-strand synthesis: First, the samples are mixed by gentle vortex, spun to collect the liquid at the bottom, and incubated in a thermocycler with the following program: 1) 4° C. for 5 min., 2) 4-37° C. for 8.25 min, (ramp rate to 0.1° C./s), 3) 37° C. for 30 min., and 4) hold at 4° C. Next, the mix is heated to 95° C. for 45 s in a thermocycler and then immediately cooled on ice using aluminum rack. Then, the plate is centrifuged at 500×g for 10 s at 15-25° C. to collect all the liquid content in the bottom and adding 2.5 μL of a freshly prepared solution of Ix Blue buffer, 0.4 mM dNTP mix, 4 μM preamplification oligo, and 10 U/μL Klenow exo- was added.

A fifth round of first-strand synthesis is performed by mixing by gentle vortex, spinning the plate to collect the liquid at the bottom, and incubating in a thermocycler with the following program: 1) 4° C. for 5 min., 2) 4-37° C. for 8.25 min, 3) 37° C. for 90 min., and 4) hold at 4° C.

Following preamplification, the samples are treated with Exonuclease I by adding 2 μL of Exonuclease I and 48 μL of Molecular BioGrade Water. The samples are incubated in the thermocycler at 37° C. for 1 hour with the lid temperature set to 50° C. At this point, the IS strand product can be stored at 4° C. overnight or at −20° C. for at least 1 month.

The preamplified samples are then purified by washing the DNA using AMPure XP beads. A volume of 64 μL (0.8×) of Ampure XP beads are added to each sample, mixed by pipetting up and down and incubated at room temperature for 10 min. The plate is placed on a magnet for 3 minutes or until the solution cleared and the supernatant is removed and discarded. The plate is removed from the magnet and 200 μL of 80% (vol/vol) ethanol is added for a first wash. The sample is mixed gently by pipetting up and down twice. The plate is returned to the magnet, and, once the beads have pelleted, the supernatant is removed. A second wash with 200 μL of 80% (vol/vol) ethanol is performed following the same procedure, and the supernatant is removed after pelleting the beads using the magnet. The AMPure XP beads are dried for 5-10 min. at room temperature and resuspended with 49 μL of an adapter oligo mix (final concentration of Ix Blue buffer, 0.4 mM dNTP mix, 0.4 μM adapter oligo).

### Adapter Tagging and Purification of Double-Tagged Products

The resuspended AMPure XP beads in the adapter oligo mix are incubated 10 min. at RT to elute the DNA from the beads. Next, they are heated to 95° C. for 45 s in a thermocycler, and immediately cooled on ice using an aluminum rack. The PCR plate is spun down at 500×g for 10 s at 15-25° C. to collect liquid at the bottom. Then, 1 μL of Klenow exo- (50 U/μl stock) is added to each sample, and the plate is vortexed gently and spun down at 500×g for 10 s at 15-25° C. and incubated in a thermocycler with the following program: 1) 4° C. for 5 min., 2) 4-37° C. for 8.25 min. (ramp rate of 0.1° C./s), 3) 37° C. for 90 min., and 4) hold at 4° C.

Following Adapter Tagging, the double-tagged products are purified, A PEG buffer (18% PEG 8,000, 2.5 M NaCl, 10 mM Tris-HCl (pH 8.0), 1 mM EDTA and 0.05% (vol/vol) Tween 20), is equilibrated at room temperature for 30 min. Next, 50 μL of elution buffer (EB) and 80 μL of PEG buffer are added to the adaptor tagging product and AMPure XP beads, mixed by pipetting up and down 10 times, and incubated for 10 min. at room temperature. The mixture is placed on a magnet for 3 min. or until the solution clears, and the supernatant is removed and discarded. The mixture is removed from the magnet and 200 μL of 80% (vol/vol) ethanol is added. The mixture is mixed gently by pipetting up and down twice and returned to the magnet. The supernatant is removed and the beads AMPure XP are washed again with 200 μL of 80% (vol/vol) ethanol on the magnet. The supernatant is removed and the beads were dried for 5-10 min. at room temperature. The AMPure XP beads are resuspended in 38 μL of a PCR reaction mix (final concentration of ix KAPA HiFi Fidelity Buffer GXL Buffer, 0.2 mM GXL dNTPs, 0.02 U/μuL KAPA HiFi HotStart polymerase).

### Library Amplification and Purification

The resuspended mixture in the PCR reaction mix is incubated at room temperature for 10 min. to elute the DNA. Added 1 μL of 10 μM PE1.0 oligo and 1 μl of different, cell-specific iPCRTag 10 μM primer to each well. The plate is scaled and mixed by gentle vortex. The libraries are amplified with the thermal profile shown in Table 7.

TABLE 7
Thermal profile for single cell methylome
sequencing library amplification

| | Number of | | |
|---|---|---|---|
| Segment | cycles | Temperature (° C.) | Duration (s) |
| 1 | 1 | 98° C. | 180 |
| 2 | 10-14 | 98° C. | 15 |
| | | 60° C. | 30 |
| | | 68° C. | 30 |
| 3 | 1 | 68° C. | 180 |
| 4 | 1 | 4° C. | Hold |

The libraries are then purified following amplification. A PEG buffer is equilibrated at room temperature for 30 mini Next, 50 μL of elution buffer (EB) and 80 μL of PEG buffer are added to each amplified library. The mixture is mixed by pipetting up and down 10 times and incubated for 10 min. at room temperature. The libraries are then transferred to a polypropylene, 96-deep well plate. On a magnet, the beads are washed twice with 200 μL of 80% (vol/vol) ethanol. The ethanol is removed and the beads are dried on a magnet for 10 min. Then, 17.5 μL of EB buffer is added and the beads are mixed by gently vortex ng. The plate is removed from the magnet, and the mixture is incubated at room temperature for 10 min.

The plate is placed on the magnet for 2 min. or until the solution cleared. 15.5 μL of each library was then transferred to a new Eppendorf loBind 96-well PCR plate. The size distribution and potential presence of adapter dimers of each library are verified by digital electrophoresis with the Fragment Analyzer system, using the HS NGS Fragment Kit (1-6000 bp) Kit. Each library is quantified with qPCR and diluted down to 4 nM. Equal volumes are pooled into a single aliquot, which is then spiked with 15% of Phix Control V3 and sequenced in an Illumina platform using V2 chemistry, 2×76 bp, to verify library mapping rates and bisulfite conversion efficiency.

The libraries with mapping rates between 20-60% are then sequenced into NextSeq 2000, providing 25-50 million reads per library.

### Example 11: Preparing Epigenetic Maps from Combined Long-Read Sequencing Reads and Single-Cell Methylome Sequencing Reads

In this example, epigenetic maps are prepared from sequencing reads from long-read sequencing according to the methods described in Examples 1-3 and from single-cell methylome sequencing according to the methods described in Example 10. Collectively, the epigenetic maps generated from the reads have the advantage of long-read sequencing by providing detailed local contiguous methylation information at a single nucleotide resolution and the advantage from single-cell methylome sequencing by representing the methylome at a single-cell resolution. This method is applied to sequence bulk cell samples comprising mixtures of different cell/tissue types and blood samples comprising cell-free DNA to generate epigenetic maps that represent the whole methylome of many different cell types at a single-nucleotide and single-cell resolution,

What is claimed is:

1. An epigenetic profiling method, comprising:
obtaining DNA molecules from a cell population;
sequencing the DNA molecules to provide a plurality of sequence reads comprising a methylation status for a plurality of bases in each sequence read; and
assembling, using one or more processors, a plurality of contigs based on the plurality of sequence reads, wherein sequence reads having the same nucleobase sequence and methylation statuses within overlapping portions are joined together to form the same contig, and wherein contigs having substantially the same nucleobase sequence and different methylation profiles are identified as being associated with different cells in the cell population.

2. The method of claim 1, wherein the sequence reads in the plurality of sequence reads are about 1000 to about 100,000 bases in length.

3. The method of claim 1 or 2, wherein the sequencing comprises a direct determination of methylation status for bases in the DNA molecules.

4. The method of claim 3, wherein the method comprises nanopore sequencing.

5. The method of claim 3, wherein the sequencing comprises a determination of methylation status based on polymerase kinetics.

6. The method of any one of claims 1-5, wherein the method does not comprise bisulfite treatment of the DNA molecules.

7. The method of any one of claims 1-6, wherein the contigs comprise substantially complete chromosomes.

8. The method of any one of claims 1-7, wherein assembling the plurality of contigs comprises using the methylation status for a plurality of CpG sites in the sequence reads.

9. The method of any one of claims 1-8, wherein assembling a contig in the plurality of contigs comprises matching a nucleobase sequence and methylation statuses in overlapping portions of at least two sequence reads.

10. The method of claim 9, wherein assembling a contig in the plurality of contigs comprises matching a sequence and methylation statuses in overlapping portions of at least three sequence reads.

11. The method of any one of claims 1-10, wherein the sequencing comprises directly determining a 5mC status, a 5hmnC status, or a 6 mA status of one or more bases in the DNA molecules.

12. The method of any one of claims 1-11, wherein cells in the cell population are undergoing cellular reprogramming or have been subject to cellular reprogramming.

13. The method of claim 12, wherein the cellular reprogramming comprises contacting the cells with one or more cellular reprogramming factors that modify one or more epigenetic markers.

14. The method of claim 13, wherein the one or more cellular reprogramming factors targets one or more epigenetic markers.

15. The method of claim 14, wherein the one or more cellular reprogramming factors that target the one or more epigenetic markers are targeted using a nuclease-deficient targeted DNA binding protein.

16. The method of claim 14 or 15, wherein the one or more cellular reprogramming factors that target the one or more target epigenetic markers are targeted using a CRISPR-based editing platform.

17. The method of claim 16, wherein the CRISPR-based editing platform of the one or more cellular reprogramming factors comprises one or more single guide RNA (sgRNA) molecules that targets one or more epigenetic markers.

18. The method of claim 16 or 17, wherein the CRISPR-based editing platform of the one or more cellular reprogramming factors comprises a dead Cas9 endonuclease.

19. The method of claim 15, wherein the nuclease-deficient targeted DNA binding protein of the one or more cellular reprogramming factors comprises a transcription activator-like (TAL) effector DNA-binding domain or a zinc finger DNA binding domain.

20. The method of any one of claims 13-19, wherein the one or more cellular reprogramming factors comprises one or more non-targeted cellular reprogramming factors.

21. The method of claim 20, wherein the one or more non-targeted cellular reprogramming factors comprises one or more transcription factors.

22. The method of claim 21, wherein the one or more transcription factors comprises one or more Yamanaka factors.

23. The method of any one of claims 20-22, wherein the one or more non-targeted cellular reprogramming factors comprises a high potassium medium.

24. The method of any one of claims 13-23, wherein the one or more cellular reprogramming factors comprises an epigenetic modification enzyme or an effector that recruits an epigenetic modification enzyme.

25. The method of any one of claims 13-24, wherein the one or more cellular reprogramming factors comprises KRAB, VPR, p65 VP64, HSF1, p300, DNMT3A, TET1, EZH2, G9a SUV39H1, HDAC3, LSD1, PRDM9, DOT1L, FOG1, BAF, PYL1, ABI1, CIBN, ADAR2, METTL3, METTL14, ALKBH5, or FTO, or an active fragment thereof.

26. The method of any one of claims 12-25, wherein the cellular reprogramming comprises contacting the cells with a blocking reagent that specifically binds to one or more epigenetic markers.

27. The method of claim 26, wherein the blocking reagent comprises a nuclease-deficient targeted DNA binding protein.

28. The method of claim 27, wherein the blocking reagent comprises nuclease-deficient targeted DNA binding protein that does not comprise a cellular reprogramming factor.

29. The method of claim 28, wherein the blocking reagent comprises nuclease-deficient CRISPR-based editing platform.

30. The method of claim 29, wherein the CRISPR-based editing platform of the blocking reagent comprises one or more single guide RNA (sgRNA) molecules that targets one or more epigenetic markers.

31. The method of claim 29 or 30, wherein the CRISPR-based editing platform of the blocking reagent comprises a dead Cas9 endonuclease.

32. The method of claim 27, wherein the nuclease-deficient targeted DNA binding protein of the blocking reagent comprises a transcription activator-like (TAL) effector DNA-binding domain or a zinc finger DNA binding domain.

33. A method of evaluating a cell, comprising:
obtaining an epigenetic profile for the cell in the cell population according to the method of any one of claims 1-32; and
determining a differential between the obtained epigenetic profile and a target epigenetic profile.

34. The method of claim 33, where the target epigenetic profile comprises one or more target epigenetic markers, and wherein the one or more cellular reprogramming factors targets the one or more target epigenetic markers.

35. The method of claim 34, wherein the one or more target epigenetic markers comprises an epigenetic marker associated with a biological age or a disease state.

36. The method of any one of claims 1-35, wherein the cells in the population of cells are obtained from a cell line.

37. The method of any one of claims 1-35, wherein the cells in the population of cells are obtained from a tissue sample from an individual.

38. The method of any one of claims 1-37, wherein the cells in the population of cells comprise fibroblasts, keratinocytes, peripheral mononuclear blood cells, hepatocytes, neural cells, blood cells, immune cells, lung cells, pancreatic beta cells, cardiomyocytes, oligodendrocytes, or epithelial cells.

39. The method of claim 38, wherein the cells in the population of cells comprise pancreatic beta cells or pancreatic alpha cells.


SEQUENCE LISTING (23 sequences)

SEQ ID NO: 1 - RNA, 19 nt, synthetic construct
  source 1..19: mol_type = other RNA
ccggactcggacgcgtggt

SEQ ID NO: 2 - RNA, 19 nt, synthetic construct
  source 1..19: mol_type = other RNA
tgtccagggacaatgagca

SEQ ID NO: 3 - RNA, 19 nt, synthetic construct
  source 1..19: mol_type = other RNA
gacaatgagcagggtgtcc

SEQ ID NO: 4 - RNA, 19 nt, synthetic construct
  source 1..19: mol_type = other RNA
gagagcgagcgcgcaacgg

SEQ ID NO: 5 - RNA, 19 nt, synthetic construct
  source 1..19: mol_type = other RNA
ggcctggcaggatgcgcgg

SEQ ID NO: 6 - RNA, 19 nt, synthetic construct
  source 1..19: mol_type = other RNA
ccgaaacgcgccaagttgg

SEQ ID NO: 7 - DNA, 7041 bp, synthetic construct
  source 1..7041: mol_type = other DNA
atgaaccacgaccaagagttcgacccgcctaaggtgtaccctcctgtgcctgccgagaagcggaagcctatcagagtgctgagcctgttcgacggaatcgccacaggactgctggtgctgaaggatctgggaatccaggtggaccggtatatcgccagcgaagtgtgcgaggacagcatcacagttggcatggttcgacaccagggcaagattatgtacgtgggcgacgtgcggagcgtgacccagaaacacattcaagagtggggccccttcgacctggttatcggcggcagcccttgcaacgatctgagcatcgtgaaccccgccagaaagggcctgtatgaaggcaccggcagactgttcttcgagttctacagactgctgcacgacgccagacctaaagagggcgacgacagaccattcttctggctgttcgagaacgtggtggccatgggcgtgtccgacaagcgggatatcagccggttcctggaaagcaaccccgtgatgatcgacgccaaagaggtgtccgctgctcaccgggccagatacttttggggcaatctgcccggcatgaacagacctctggccagcaccgtgaacgacaagctggaactgcaagagtgcctggaacacggccggatcgccaagtttagcaaagtgcggaccatcaccaccaggtccaacagcatcaagcagggcaaagaccagcacttccccgtgttcatgaatgagaaagaggacatcctgtggtgcaccgagatggaaagagtgttcggcttccctgtgcactacaccgacgtgtccaacatgagcagactggccagacagaggctgctgggcagatcttggagcgtgccagtgatcagacacctgttcgcccctctgaaagaatacttcgcctgcgtgtccagcggcaacagcaacgccaattctagaggccccagcttcagctctggactggtgccattgagcctgagaggctctcacatgggccccatggaaatctacaagaccgtgtccgcctggaagagacagcctgtcagagtcctgtctctgttccgcaacatcgacaaggtgctgaaaagcctgggcttcctggaatctggcagcggatctggcggcggaaccctgaaatacgtggaagatgtgaccaacgtcgtgcggcgcgacgtggaaaagtggggaccttttgatctggtgtacggcagcacacagccactgggcagcagctgcgatagatgtcctggctggtacatgttccagttccaccggatcctgcagtacgccctgcctagacaagagagccagcggcctttcttctggatcttcatggacaacctgctgctgaccgaggacgatcaagagacaaccaccagatttctgcagaccgaggccgtgacactccaggatgttcggggcagagactaccagaacgccatgagagtgtggtctaacatccccggcctgaagtccaaacacgccccactgacacccaaagaggaagagtacctgcaggcccaagtgcggagcagatccaaactggatgcccctaaggtggacctgctggtcaagaactgtctgctgcccctgagagagtacttcaagtatttcagccagaacagcctgcctctcggcggaccttctagcggagcacctcctccaagcggaggatctcctgctggaagccctacctctaccgaggaaggcacaagcgagtctgccacacctgagtctggccctggcacatctacagagcctagcgaaggttctgcccctggatctccagccggctctcctacatctactgaagagggcaccagcaccgagccatctgaaggcagtgcacctggcacaagcacagagccctccgaaatggacaagaagtacagcatcggcctggccatcggcaccaattctgttggatgggccgtgatcaccgacgagtacaaggtgcccagcaagaagttcaaggtcctgggcaacaccgaccggcactccatcaagaagaatctgatcggcgccctgctgttcgactctggcgaaacagccgaagccaccagactgaagagaaccgccagaaggcgctacacccggcggaagaaccggatctgttacctgcaagagatcttcagcaacgagatggccaaggtggacgacagcttcttccacagactggaagagtccttcctggtggaagaggataagaagcacgagcggcaccccatcttcggcaacatcgtggatgaggtggcctaccacgagaagtaccccaccatctaccacctgagaaagaaactggtggacagcaccgacaaggccgacctgagactgatctatctggctctggcccacatgatcaagttccggggccactttctgatcgagggcgatctgaaccccgacaactccgacgtggacaagctgttcatccagctggtgcagacctacaaccagctgttcgaagagaatcccatcaacgcctctggcgtggacgccaaggctatcctgtctgccagactgagcaagagcagacggctggaaaacctgatcgctcagctgccaggcgagaagaagaacggcctgttcggcaacctgattgccctgagcctgggcctgacacctaacttcaagagcaacttcgatctggccgaggatgccaaactgcagctgtccaaggacacctacgacgacgacctggataatctgctggcccagatcggcgaccagtacgccgacttgtttctggccgccaagaacctgtccgacgccatcctgctgagcgacattctgagagtgaacaccgagatcaccaaggcacctctgagcgccagcatgattaagagatacgacgagcaccatcaggatctgaccctgctgaaggccctcgtcagacagcagctcccagagaagtacaaagagattttcttcgaccagagcaagaacggctacgccggctacattgatggcggagccagccaagaggaattctacaagttcatcaagcccatcctcgagaagatggacggcaccgaggaactgctcgtgaagctgaacagagaggatctgctgagaaagcagaggaccttcgacaacggcagcatccctcaccagatccacctgggagaactgcacgccattctgcggagacaagaggacttttacccattcctgaaggacaaccgggaaaagattgagaagatcctgaccttcaggatcccctactacgtgggaccactggccaggggcaatagcagatttgcctggatgaccagaaagagcgaggaaaccatcacaccctggaacttcgaggaagtggtggataagggcgccagcgctcagtccttcatcgagcggatgaccaacttcgacaagaatctgcctaacgagaaggtgctgcccaagcacagcctgctgtacgagtactttaccgtgtacaacgagctgaccaaagtcaaatacgtgaccgaggggatgagaaagcccgcctttctgagcggcgagcagaaaaaggccatcgtcgatctgctgttcaagaccaaccggaaagtgaccgtgaagcagctgaaagaggattacttcaagaaaatcgagtgcttcgacagcgtggaaatcagcggcgtcgaggacagattcaatgccagcctgggcacataccacgacctgctgaaaattatcaaggacaaggacttcctggacaacgaggaaaacgaggacattcttgaggacatcgtgctgaccctgacactgttcgaggatagagagatgatcgaggaacggctgaaaacatacgcccacctgtttgacgacaaagtgatgaagcaactgaagcggcggagatacaccggctggggtagactgtcccggaagctgatcaacggcatccgggataagcagtccggcaagaccatcctggactttctgaagtccgacggcttcgccaacagaaacttcatgcagctgatccacgacgacagcctgacctttaaagaggatatccagaaggcccaggtttccggccagggcgattctctgcacgagcacattgccaatctggccggatctcccgccattaagaagggcatcctgcagacagtgaaggtggtggatgagctggtcaaagtcatgggcagacacaagcccgagaacatcgtgatcgaaatggccagagagaaccagaccacacagaagggacagaagaacagccgcgagagaatgaagcggatcgaagagggaatcaaagagctgggcagccagatcctcaaagaacaccccgtggaaaacacccagctgcagaacgagaagctgtacctgtactacctgcagaatggacgggatatgtacgttgaccaagagctggacatcaaccggctgtccgactacgatgtggacgctatcgtgccccagagcttcctgaaagacgactccatcgacaacaaagtgctgactcggagcgacaagaaccggggcaagagcgacaatgtgccaagcgaagaggtcgtgaagaagatgaagaactactggcgacagctgctgaatgccaagctgattacccagcggaagttcgacaatctgaccaaggccgagagaggcggcctgagcgaacttgataaggccggcttcattaagcggcagctggtggaaacccggcagatcacaaaacacgtggcacagatcctggatagccggatgaacactaagtacgacgagaacgacaaactgatccgggaagtgaaagtgatcaccctcaagtccaagctggtgtccgatttccggaaggatttccagttctacaaagtccgcgagatcaacaactaccatcacgcccacgacgcctacctgaatgccgttgttggaacagccctgatcaagaagtatcccaagctcgagagcgagttcgtgtacggcgactacaaggtgtacgatgtgcgcaagatgattgccaagagcgagcaagaaatcggcaaggctaccgccaagtacttcttctacagcaacatcatgaatttcttcaagacagagatcacgctggccaacggcgagatccggaaaagacccctgatcgagacaaacggcgaaaccggggagatcgtgtgggacaagggcagagattttgccaccgtgcggaaagtgctgtctatgcctcaagtgaacatcgtcaaaaagaccgaggtgcagacaggcggcttcagcaaagagtctatcctgcctaagaggaacagcgataagctgatcgcccggaagaaggactgggaccctaagaagtacggcggcttcgatagccctaccgtggcctattctgtgctggtggtggcaaaggtggaaaagggcaagtccaaaaagctcaagagcgtgaaagagctgctggggatcaccatcatggaaagaagcagcttcgagaagaacccgatcgactttctggaagccaagggctacaaagaagtcaagaaggacctgatcatcaagctccccaagtactccctgttcgagctggaaaatggccggaagcggatgctggcttctgctggcgaactgcagaagggaaacgaactggccctgccttccaaatatgtgaacttcctgtacctggccagccactatgagaagctgaagggcagccccgaggacaatgagcaaaagcagctgtttgtggaacagcacaaacactacctggacgagatcatcgagcagatcagcgagttctccaagagagtgatcctggccgacgctaacctggacaaagtcctgagcgcctacaacaagcaccgggacaagccaatcagagagcaggccgagaatatcatccacctgttcaccctgaccaacctgggagcccctgccgccttcaagtactttgataccaccatcgaccggaagaggtacacctccacaaaagaggtgctggacgccacactgatccaccagtctatcaccggcctgtacgagacacggatcgacctgtctcagcttggaggcgacgcctatccttacgacgtgccagactatgctagcctcggctctggcagccccaagaagaagagaaaggtcgaggaccccaagaaaaagcgcaaagtggacggcatcggcagcggctctaatggctctagcggaagcagcgaactgatcaaagaaaacatgcacatgaagctctacatggaaggcactgtggacaatcaccacttcaagtgcaccagcgaaggcgagggcaagccttatgagggcacccagaccatgcggatcaaggtggtcgaaggcggacctctgcctttcgccttcgatatcctggctaccagctttctgtacggctccaagaccttcatcaatcacacccagggcatccccgactttttcaagcagagctttcccgagggcttcacttgggagagagtgaccacatacgaggacggcggagtgctgacagccacacaggatacaagcctgcaggacggctgcctgatctacaacgtgaagatccggggcgtgaacttcaccagcaacggccctgtgatgcagaagaaaaccctcggctgggaagccttcaccgagacactgtatccagccgatggcggactggaaggccggaatgatatggccctgaagctcgttggcggctcccatctgatcgccaacatcaagaccacctacagaagcaagaagcccgctaagaacctcaagatgcccggcgtgtactatgtggactaccggctggaacgcatcaaagaggccaacaacgaaacctacgtcgagcagcacgaagtggccgtggccagatattgcgacctgccttctaagctgggccacaagttgaatggcggaggcggaggaatggacgctaagagccttaccgcctggtctagaaccctggtcaccttcaaggacgtgttcgtggacttcacccgggaagagtggaagctgctggacacagcacagcagattgtgtaccggaacgtgatgctcgagaactacaaaaacctggtgtccctgggctatcagctgacaaagcccgacgtgatcctgcgcctggaaaaaggcgaagaaccctag

SEQ ID NO: 8 - RNA, 100 nt, synthetic construct
  source 1..100: mol_type = other RNA
ccggactcggacgcgtggtgttttagagctagaaatagcaagttaaaataaggctagtccgttatcaacttgaaaaagtggcaccgagtcggtgcttttt

SEQ ID NO: 9 - RNA, 100 nt, synthetic construct
  source 1..100: mol_type = other RNA
tgtccagggacaatgagcagttttagagctagaaatagcaagttaaaataaggctagtccgttatcaacttgaaaaagtggcaccgagtcggtgcttttt

SEQ ID NO: 10 - RNA, 100 nt, synthetic construct
  source 1..100: mol_type = other RNA
gacaatgagcagggtgtccgttttagagctagaaatagcaagttaaaataaggctagtccgttatcaacttgaaaaagtggcaccgagtcggtgcttttt

SEQ ID NO: 11 - RNA, 100 nt, synthetic construct
  source 1..100: mol_type = other RNA
gagagcgagcgcgcaacgggttttagagctagaaatagcaagttaaaataaggctagtccgttatcaacttgaaaaagtggcaccgagtcggtgcttttt

SEQ ID NO: 12 - RNA, 100 nt, synthetic construct
  source 1..100: mol_type = other RNA
ggcctggcaggatgcgcgggttttagagctagaaatagcaagttaaaataaggctagtccgttatcaacttgaaaaagtggcaccgagtcggtgcttttt

SEQ ID NO: 13 - RNA, 100 nt, synthetic construct
  source 1..100: mol_type = other RNA
ccgaaacgcgccaagttgggttttagagctagaaatagcaagttaaaataaggctagtccgttatcaacttgaaaaagtggcaccgagtcggtgcttttt

SEQ ID NO: 14 - protein, 2346 aa, synthetic construct
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDAYPYDVPDYASLGSGSPKKKRKVEDPKKKRKVDGIGSGSNGSSGSSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLNGGGGGMDAKSLTAWSRTLVTFKDVFVDFTREEWKLLDTAQQIVYRNVMLENYKNLVSLGYQLTKPDVILRLEKGEEP

SEQ ID NO: 15 - RNA, 81 nt, synthetic construct
  source 1..81: mol_type = other RNA
gttttagagctagaaatagcaagttaaaataaggctagtccgttatcaacttgaaaaagtggcaccgagtcggtgcttttt

SEQ ID NO: 16 - protein, 7 aa, synthetic construct
PKKKRKV

SEQ ID NO: 17 - protein, 25 aa, synthetic construct
MSRRRKANPTKLSENAKKLAKEVEN

SEQ ID NO: 18 - protein, 9 aa, synthetic construct
KLKIKRPVK

SEQ ID NO: 19 - protein, 8 aa, synthetic construct
VSRKRPRP

SEQ ID NO: 20 - protein, 10 aa, synthetic construct
EGAPPAKRAR

SEQ ID NO: 21 - protein, 16 aa, synthetic construct
RQARRNRRRRWRERNR

SEQ ID NO: 22 - protein, 12 aa, synthetic construct
PPQPKKKPLDGE

SEQ ID NO: 23 - protein, 20 aa, synthetic construct
AVKRPAATKKAGQAKKKKLD